(19) United States
(12) Patent Application Publication
(10) Pub. No.: US 2004/0197345 A1
(43) Pub. Date: Oct. 7, 2004

(54) Prostate derived Ets factor

(75) Inventors: Towia Aron Libermann, Newton, MA (US); Joerg Peter Oettgen, Brookline, MA (US); Charles A. Kunsch, Norcross, GA (US); Gregory A. Endress, Florence, MA (US); Craig A. Rosen, Laytonsville, MD (US)

Correspondence Address: HUMAN GENOME SCIENCES INC, INTELLECTUAL PROPERTY DEPT., 14200 SHADY GROVE ROAD, ROCKVILLE, MD, 20850, US

(73) Assignees: Human Genome Sciences, Inc., Rockville, MD (US); Beth Israel Deaconess Medical Center, Boston, MA (US)

(21) Appl. No.: 10/838,371

(22) Filed: May 5, 2004

Related U.S. Application Data
(63) Continuation of application No. 09/126,945, filed on Jul. 31, 1998, now abandoned.

(51) Int. Cl. A61K 48/00; C07H 21/04; A61K 39/00

(52) U.S. Cl. 424/185.1; 435/69.3; 435/320.1; 435/325; 530/350; 536/23.5; 514/44

(57) ABSTRACT

The present invention relates to a novel human protein called Prostate Derived Ets Factor, and isolated polynucleotides encoding this protein. Also provided are vectors, host cells, antibodies, and recombinant methods for producing this human protein. The invention further relates to diagnostic and therapeutic methods useful for diagnosing and treating disorders related to this novel human protein.

### CROSS REFERENCE TO RELATED APPLICATIONS

This application is a continuation of U.S. application Ser. No. 09/126,945, filed Jul. 31, 1998, which is hereby incorporated by reference.

### FIELD OF THE INVENTION

The present invention relates to a novel human gene encoding a polypeptide which is a member of the Ets family. More specifically, the present invention relates to a polynucleotide encoding a novel human polypeptide named Prostate Derived Ets Factor, or “PDEF.” This invention also relates to PDEF polypeptides, as well as vectors, host cells, antibodies directed to PDEF polypeptides, and the recombinant methods for producing the same. Also provided are diagnostic methods for detecting disorders related to the reproductive system, and therapeutic methods for treating such disorders. The invention further relates to screening methods for identifying agonists and antagonists of PDEF activity.

### BACKGROUND OF THE INVENTION

Epithelial cell differentiation is regulated by the combined action of growth factors, cytokines, cell-cell interactions, and a distinct set of transcription factors which, by binding to the regulatory regions of a gene, modulate and coordinate developmental stage-specific and lineage-specific gene expression.

Analysis of the regulatory regions of epithelial cell-specific genes reveals the presence of repeated DNA motifs which function as binding sites for transcription factors. For example, many epithelial cell-specific genes contain motifs that bind transcription factors, such as Ets factors, SP1, TTF-1, LEF-1, retinoic acid receptors, AP-1, AP-2, LFB3, KDF-1, Oct-6, and skn-1a. However, very few of these transcription factors are expressed solely in epithelial cells, suggesting that either these different transcription factors cooperate with one another, resulting in selective stage- and cell-specific expression of a particular epithelial gene, or that unknown epithelial-specific transcription factors interact with these factors to turn on the expression of epithelial-specific genes.

The Ets transcription factor gene family contains more than twenty members, which have been shown to transform cells when aberrantly expressed. All Ets factors share a highly conserved DNA binding domain, the Ets domain, which recognizes a core binding motif, of sequence “A/GGAA/T” within the promoter regions of Ets stimulated genes. Despite the conservation of this motif, slight variability exists within the flanking nucleotide positions. Although Ets factors can be grouped into subclasses based on additional homologous domains unique to particular members of the Ets family, very little homology exists between the Ets family members outside of the DNA binding domain.

Ets factors play a critical role in the transcriptional control of stringently regulated genes, such as genes involved in tissue-development, differentiation, angiogenesis, cell cycle control, and cell proliferation, acting as either transcriptional enhancers or repressors. However, relatively little is known about the role of Ets factors in epithelial cells.

Thus, there is a need for polypeptides that regulate epithelial specific gene expression, since disturbances of such regulation may be involved in hyperproliferative disorders, including cancer, particularly of the reproductive system. Furthermore, there is a need for identifying and characterizing such human polypeptides which may play a role in the detection, prevention, and/or correction of such disorders.

### SUMMARY OF THE INVENTION

The present invention relates to novel polynucleotides and the encoded polypeptides of PDEF. Moreover, the present invention relates to vectors, host cells, antibodies, and recombinant methods for producing the polypeptides and polynucleotides. Also provided are diagnostic methods for detecting disorders related to the polypeptides, and therapeutic methods for treating such disorders. The invention further relates to screening methods for identifying binding partners of PDEF.

### BRIEF DESCRIPTION OF THE DRAWINGS

FIGS. 1A-B show the nucleotide sequence (SEQ ID NO:1) and the deduced amino acid sequence (SEQ ID NO:2) of PDEF. Regions of conservation to the pointed and Ets domains are indicated by single and double underline, respectively.

FIG. 2 shows the regions of identity between the amino acid sequence of the PDEF protein and the translation product of the Drosophila melanogastor ETS-4 (SEQ ID NO:3), (Genebank Accession No. gi|157196), determined by Megalign (DNA Star suite of programs) analysis. Identical amino acids between the two polypeptides are shaded, while conservative amino acid are boxed. By examining the regions of amino acids shaded and/or boxed, the skilled artisan can readily identify conserved domains between the two polypeptides.

FIG. 3 shows an analysis of the PDEF amino acid sequence. Alpha, beta, turn and coil regions; hydrophilicity and hydrophobicity; amphipathic regions; flexible regions; antigenic index and surface probability are shown. In the “Antigenic Index or Jameson-Wolf” graph, the positive peaks indicate locations of the highly antigenic regions of the PDEF protein, i.e., regions from which epitope-bearing peptides of the invention can be obtained. The domains defined by these graphs are contemplated by the present invention. Tabular representation of the data summarized graphically in FIG. 3 can be found in Table 1.

FIGS. 4A-C show the tissue distribution of PDEF expression in different human fetal (FIG. 4A) and adult tissues (FIGS. 4B-C) by Northern hybridization. The blots were sequentially probed with PDEF (upper panel), ESE-1 (middle panel) and GADPH cDNA probes (lower panel) under stringent conditions using poly(A)+ mRNA from the indicated tissues (See Example 3 of present invention). A skilled artisan would readily associate the intensity and location of the bands with respect to the blot as indicative of both the abundance and size of the PDEF mRNA within each tissue.

FIGS. 5A-B show the tissue distribution of PDEF expression within poly(A)+ mRNA from human fetal and adult tissues by Dot Blot Hybridization. The blot was probed with PDEF under the conditions described in Example 3 of present invention. A skilled artisan would readily associate the intensity of the dots as indicative of both the abundance of the PDEF mRNA within each tissue.

FIGS. 6A-D show in situ hybridization studies. Paired brightfield (A,C) and corresponding polarized fluorescence (B,D) photomicrographs. Intense labeling of prostate epithelium in normal lung is seen with antisense probe to PDEF mRNA (A,B). No labeling is seen with control sense probe (C,D).

FIG. 7 shows the level of transcriptional induction of the PSA promoter in the presence of expressed PDEF polypeptides. Data shown are means of triplicate measurements from one representative transfection.

FIG. 8 illustrates GST/PDEF fusion proteins constructed.

FIG. 9 shows the in vitro translation products of unprogrammed reticulocyte lysates (indicated by an ‘−’), in addition to the full-length and truncated (Δ194) PDEF polypeptides. Translation products were separated by SDS-PAGE and visualized by autoradiography.

FIG. 10 shows that PDEF physically interacts with the androgen receptor as determined by “GST-pull down” experiments.

FIG. 11 shows that the PDEF and androgen receptor cooperate in PSA promoter activation. CV-1 cells were cotransfected with the PDEF and androgen receptor expression vectors or the parental pCI expression vector and luceriferase constructs containing the PSA promoter. Luciferase activity in the lysates was determined 48 h later in the absence or presence of androgen (DHT) as described in the examples of present invention. Data shown are means of duplicate measurements from one representative transfection.

### DETAILED DESCRIPTION

### Definitions

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide.

As used herein, a PDEF “polynucleotide” refers to a molecule having a nucleic acid sequence contained in SEQ ID NO:1 or the cDNA contained within the clone deposited with the ATCC. For example, the PDEF polynucleotide can contain the nucleotide sequence of the full length cDNA sequence, including the 5′ and 3′ untranslated sequences, the coding region, as well as fragments, epitopes, domains, and variants of the nucleic acid sequence. Moreover, as used herein, a PDEF “polypeptide” refers to a molecule having the translated amino acid sequence generated from the polynucleotide as broadly defined.

In the present invention, the full length PDEF sequence identified as SEQ ID NO:1 was generated by searching a human EST (Expressed Sequence Tag) cDNA database for sequences homologous to known Ets members (HGS clone ID: HBZSD43RA, from a subtracted Prostate Library BPH, LIB2). Several ESTs bearing nucleotide sequence identity were predicted to encode a novel Ets-like protein. These ESTs originated from cDNA clones prepared from human prostate carcinoma cDNA libraries. A representative cDNA clone containing all or most of the sequence for SEQ ID NO:1 was deposited with the American Type Culture Collection (“ATCC”) on Jul. 27, 1998, and was given the ATCC Deposit Number 203072. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

A PDEF “polynucleotide” also includes those polynucleotides capable of hybridizing, under stringent hybridization conditions, to sequences contained in SEQ ID NO:1, the complement thereof, or the cDNA within the deposited clone. “Stringent hybridization conditions” refers to an overnight incubation at 42° C. in a solution comprising 50% formamide, 5×SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C.

Also contemplated are nucleic acid molecules that hybridize to the PDEF polynucleotides at lower stringency hybridization conditions. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37° C. in a solution comprising 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone).

Also contemplated are nucleic acid molecules that comprise a polynucleotide having a nucleotide sequence at least 95% identical to a polynucleotide fragment of SEQ ID NO:1 or a polynucleotide fragment of the cDNA sequence included in ATCC Deposit No. 203072. The present invention also relates to polypeptides that comprise an amino acid sequence at least 95% identical to a polypeptide fragment of SEQ ID NO:2 or the encoded sequence included in ATCC Deposit No. 203072.

The PDEF polynucleotide can be composed of any polyribonucleotide or polydeoxribonucleotide, which may be unmodified RNA or DNA or modified RNA or DNA. For example, PDEF polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the PDEF polynucleotides can be composed of triple-stranded regions comprising RNA or DNA or both RNA and DNA. PDEF polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

PDEF polypeptides can be composed of amino acids joined to each other by peptide bonds or modified peptide bonds, i.e., peptide isosteres, and may contain amino acids other than the 20 gene-encoded amino acids. The PDEF polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the PDEF polypeptide, including the peptide backbone, the amino acid side-chains and the amino or carboxyl termini. It will be appreciated that the same type of modification may be present in the same or varying degrees at several sites in a given PDEF polypeptide. Also, a given PDEF polypeptide may contain many types of modifications. PDEF polypeptides may be branched, for example, as a result of ubiquitination, and they may be cyclic, with or without branching. Cyclic, branched, and branched cyclic PDEF polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

“SEQ ID NO:1 ” refers to a PDEF polynucleotide sequence while “SEQ ID NO:2” refers to a PDEF polypeptide sequence.

A PDEF polypeptide “having biological activity” refers to polypeptides exhibiting activity similar, but not necessarily identical to, an activity of a PDEF polypeptide as measured in a particular biological assay, with or without dose dependency. In the case where dose dependency does exist, it need not be identical to that of the PDEF polypeptide, but rather substantially similar to the dose-dependence in a given activity as compared to the PDEF polypeptide (i.e., the candidate polypeptide will exhibit greater activity or not more than about 25-fold less and, preferably, not more than about ten-fold less activity, and most preferably, not more than about three-fold less activity relative to the PDEF polypeptide.)

### PDEF Polynucleotides and Polypeptides

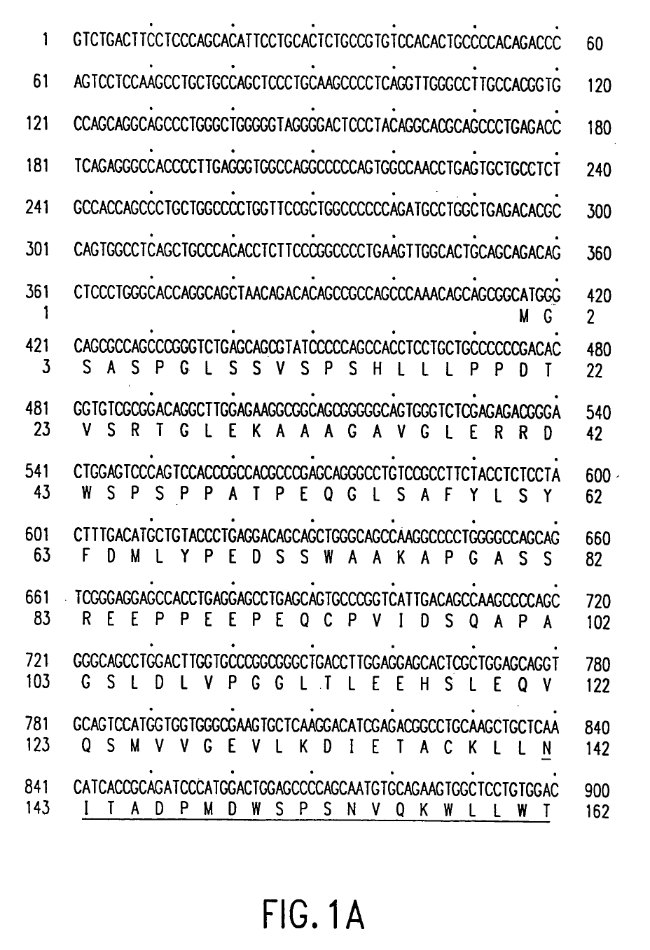

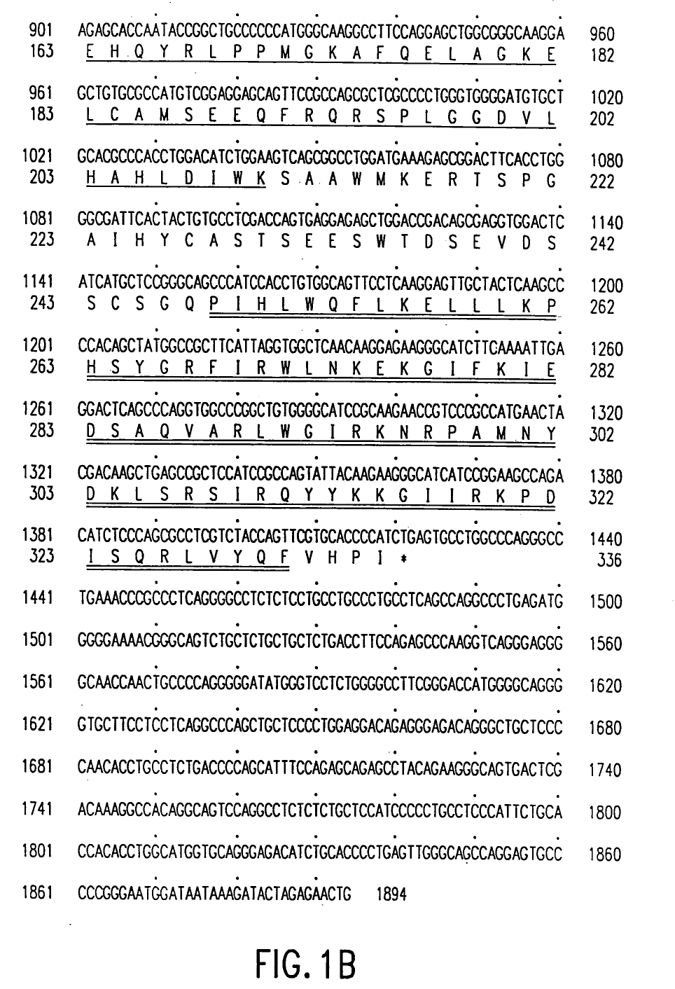

The full-length PDEF was isolated from human prostate cDNA (CLONTECH). This clone contains the entire coding region identified as SEQ ID NO:2. The deposited clone contains a cDNA having a total of 1894 nucleotides, which encodes a predicted open reading frame of 335 amino acid residues. (See FIG. 1.) The open reading frame begins at a N-terminal methionine located at nucleotide position 416, and ends at a stop codon immediately after nucleotide position 1420. The predicted molecular weight of the PDEF protein should be about 37.5 KDa.

Subsequent Northern analysis also showed PDEF expression in prostate epithelium, and to a lesser extent in salivary gland, trachea, ovary, and mammary tissues, a pattern consistent with reproductive specific expression. Expression is highest in tissues involved in androgen sensitivity consistent with the enriched expression in prostate epithelium cells. A single primary transcript of approximately 2.0 kb is observed. The expression of the major 2.0 kb transcript is highest in prostate epithelium, and, to a lesser degree, in ovary, trachea, and stomach tissues (See Example 3).

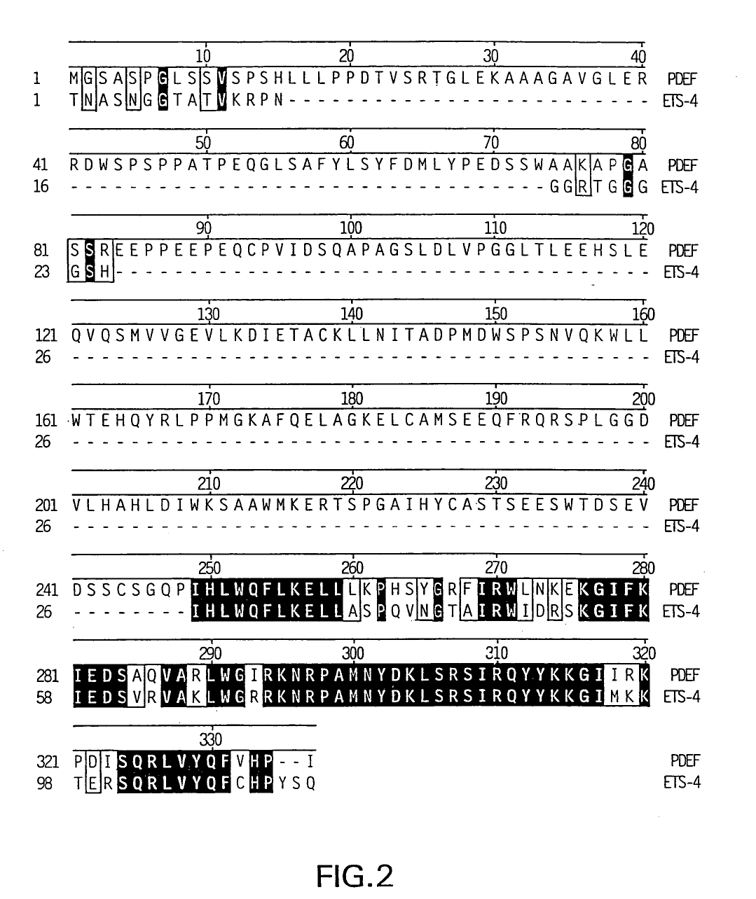

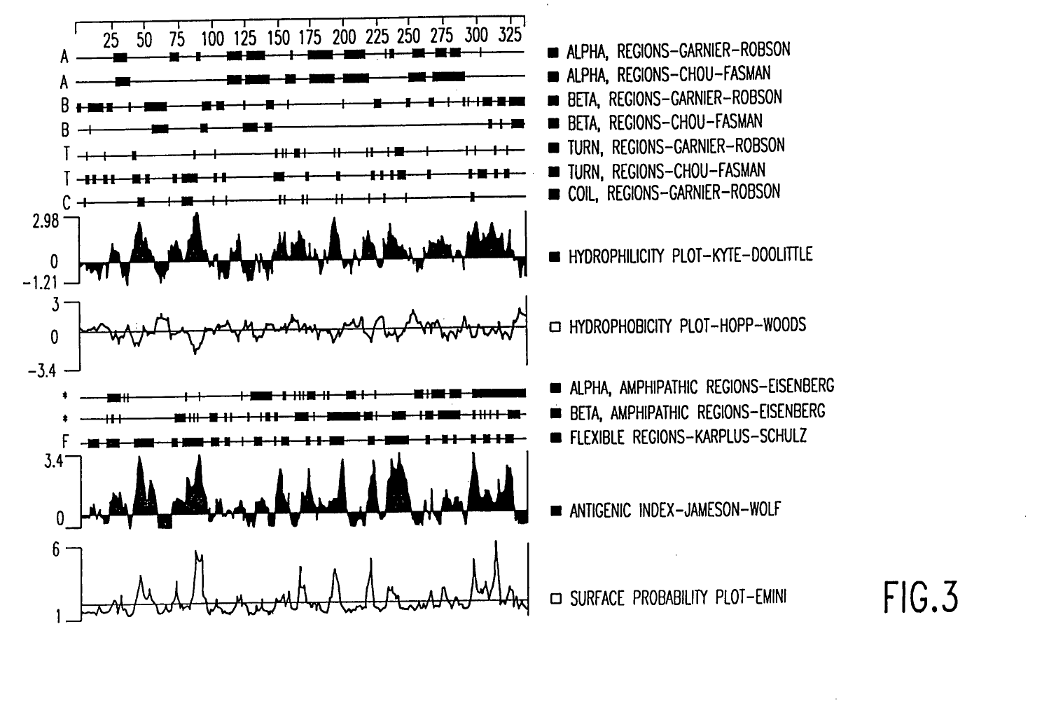

Using BLAST analysis, SEQ ID NO:2 was found to be homologous to members of the Ets family. Particularly, SEQ ID NO:2 contains domains homologous to the translation product of the Drosophila melanogaster mRNA for ETS-4 (FIG. 2) (SEQ ID NO:3), (Genebank Accession No. gi|157196), including the following conserved domains: (a) a predicted pointed domain located at about amino acids 142 to 211 of SEQ ID NO:2; and (b) a predicted Ets domain located at about amino acids 248 to 331 of SEQ ID NO:2. Polynucleotides comprising these polypeptide fragments of PDEF are specifically contemplated in the present invention. Because ETS-4 is thought to be important as a transcription factor in Drosophila melanogaster germline development, the homology between ETS-4 and PDEF suggests that PDEF may also regulate epithelial specific gene expression. The pointed domain has been found in several members of the Ets family.

Moreover, while the function of this domain is not clear, its weak homology to the HLH domain suggests it may play a role in dimerization. Other reports have shown the pointed domain's involvement in transactivation of genes.

Recently it has been demonstrated that the Ets factor tel, an Ets-related protein, can indeed dimerize via the pointed domain (Golub, T. R., Barker, G. F., Lovett, M. and Gilliland, D. G. Cell 77: 307-316 (1994)). The involvement of the tel pointed domain in various chromosomal translocations in different types of leukemias, suggests a role of the pointed domain in transformation. Thus, the pointed domain identified in PDEF is likely to play a role in dimerization and transactivation of genes.

The PDEF nucleotide sequence identified as SEQ ID NO:1 was assembled from partially homologous (“overlapping”) sequences obtained from the deposited clone, and from additional related DNA clones. The overlapping sequences were assembled into a single contiguous sequence of high redundancy (usually three to five overlapping sequences at each nucleotide position), resulting in a final sequence identified as SEQ ID NO:1.

Therefore, SEQ ID NO:1 and the translated SEQ ID NO:2 are sufficiently accurate and otherwise suitable for a variety of uses well known in the art and described further below. For instance, SEQ ID NO:1 is useful for designing nucleic acid hybridization probes that will detect nucleic acid sequences contained in SEQ ID NO:1 or the cDNA contained in the deposited clone. These probes will also hybridize to nucleic acid molecules in biological samples, thereby enabling a variety of forensic and diagnostic methods of the invention. Similarly, polypeptides identified from SEQ ID NO:2 may be used to generate antibodies which bind specifically to PDEF.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NO:1 and the predicted translated amino acid sequence identified as SEQ ID NO:2, but also a sample of plasmid DNA containing a human cDNA of PDEF deposited with the ATCC. The nucleotide sequence of the deposited PDEF clone can readily be determined by sequencing the deposited clone in accordance with known methods. The predicted PDEF amino acid sequence can then be verified from such deposits. Moreover, the amino acid sequence of the protein encoded by the deposited clone can also be directly determined by peptide sequencing or by expressing the protein in a suitable host cell containing the deposited human PDEF cDNA, collecting the protein, and determining its sequence.

The present invention also relates to the PDEF gene corresponding to SEQ ID NO:1, SEQ ID NO:2, or the deposited clone. The PDEF gene can be isolated in accordance with known methods using the sequence information disclosed herein. Such methods include preparing probes or primers from the disclosed sequence and identifying or amplifying the PDEF gene from appropriate sources of genomic material.

Also provided in the present invention are species homologs of PDEF. Species homologs may be isolated and identified by making suitable probes or primers from the sequences provided herein and screening a suitable nucleic acid source for the desired homologue.

The PDEF polypeptides can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

PDEF polypeptides are preferably provided in an isolated form, and preferably are substantially purified. A recombinantly produced version of a PDEF polypeptide, including the secreted polypeptide, can be substantially purified by the one-step method described in Smith and Johnson, Gene 67:31-40 (1988). PDEF polypeptides also can be purified from natural or recombinant sources using antibodies of the invention raised against the PDEF protein in methods which are well known in the art.

### Polynucleotide and Polypeptide Variants

“Variant” refers to a polynucleotide or polypeptide differing from the PDEF polynucleotide or polypeptide, but retaining essential properties thereof. Generally, variants are overall closely similar, and, in many regions, identical to the PDEF polynucleotide or polypeptide.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the PDEF polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown of SEQ ID NO:1, the ORF (open reading frame), or any fragment specified as described herein.

As a practical matter, whether any particular nucleic acid molecule or polypeptide is at least 90%, 95%, 96%, 97%, 98% or 99% identical to a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245.) In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identify are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to be made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequences shown in SEQ ID NO:2 or to the amino acid sequence encoded by deposited DNA clone can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty-1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to be made for the purposes of the present invention.

The PDEF variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. PDEF polynucleotide variants can be produced for a variety of reasons, e.g., to optimize codon expression for a particular host (change codons in the human mRNA to those preferred by a bacterial host such as E. coli).

Naturally occurring PDEF variants are called “allelic variants,” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of the PDEF polypeptides. For instance, one or more amino acids can be deleted from the N-terminus or C-terminus of the protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein. (Dobeli et al., J. Biotechnology 7:199-216 (1988).)

Moreover, ample evidence demonstrates that variants often retain a biological activity similar to that of the naturally occurring protein. For example, Gayle and coworkers (J. Biol. Chem. 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the protein will likely be retained when less than the majority of the residues of the protein are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes PDEF polypeptide variants which show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie, J. U. et al., Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of PDEF include (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as an IgG Fc fusion region peptide, or leader or secretory sequence, or a sequence facilitating purification. Such variant polypeptides are deemed to be within the scope of those skilled in the art from the teachings herein.

For example, PDEF polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).)

### Polynucleotide and Polypeptide Fragments

In the present invention, a “polynucleotide fragment” refers to a short polynucleotide having a nucleic acid sequence contained in the deposited clone or shown in SEQ ID NO:1. The short nucleotide fragments are preferably at least about 15 nt, and more preferably at least about 20 nt, still more preferably at least about 30 nt, and even more preferably, at least about 40 nt in length. A fragment “at least 20 nt in length,” for example, is intended to include 20 or more contiguous bases from the cDNA sequence contained in the deposited clone or the nucleotide sequence shown in SEQ ID NO:1. These nucleotide fragments are useful as diagnostic probes and primers as discussed herein. Of course, larger fragments (e.g., 50, 150, 500, 600, 2000 nucleotides) are preferred.

Moreover, representative examples of PDEF polynucleotide fragments include, for example, fragments having a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, or 1851-1894 of SEQ ID NO:1 or the cDNA contained in the deposited clone. In this context “about” includes the particularly recited ranges, larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein.

In the present invention, a “polypeptide fragment” refers to a short amino acid sequence contained in SEQ ID NO:2 or encoded by the cDNA contained in the deposited clone. Protein fragments may be “free-standing,” or comprised within a larger polypeptide of which the fragment forms a part or region, most preferably as a single continuous region. Representative examples of polypeptide fragments of the invention, include, for example, fragments from about amino acid number 1-20, 21-40, 41-60, 61-80, 81-100, 102-120, 121-140, 141-160, 161-180, 181-200, 201-220, 221-240, 241-260, 261-280, 281-300, 301-320, or 321-336 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges, larger or smaller by several (5, 4, 3, 2, or 1) amino acids, at either extreme or at both extremes.

Preferred polypeptide fragments include the PDEF protein having a continuous series of deleted residues from the amino or the carboxy terminus, or both. For example, any number of amino acids, ranging from 1-60, can be deleted from the amino terminus of the PDEF polypeptide. Similarly, any number of amino acids, ranging from 1-30, can be deleted from the carboxy terminus of the PDEF protein. Furthermore, any combination of the above amino and carboxy terminus deletions are preferred. Similarly, polynucleotide fragments encoding these PDEF polypeptide fragments are also preferred.

Particularly, N-terminal deletions of the PDEF polypeptide can be described by the general formula m-335, where m is an integer from 2 to 321 where m corresponds to the position of the amino acid residue identified in SEQ ID NO:2. Preferably, N-terminal deletions of the PDEF polypeptide of the invention shown as SEQ ID NO:2 include polypeptides comprising the amino acid sequence of residues: G-2 to I-335; S-3 to I-335; A-4 to I-335; S-5 to I-335; P-6 to I-335; G-7 to I-335; L-8 to I-335; S-9 to I-335; S-10 to I-335; V-11 to I-335; S-12 to I-335; P-13 to I-335; S-14 to I-335; H-15 to I-335; L-16 to I-335; L-17 to I-335; L-18 to I-335; P-19 to I-335; P-20 to I-335; D-21 to I-335; T-22 to I-335; V-23 to I-335; S-24 to I-335; R-25 to I-335; T-26 to I-335; G-27 to I-335; L-28 to I-335; E-29 to I-335; K-30 to I-335; A-31 to I-335; A-32 to I-335; A-33 to I-335; G-34 to I-335; A-35 to I-335; V-36 to I-335; G-37 to I-335; L-38 to I-335; E-39 to I-335; R-40 to I-335; R-41 to I-335; D-42 to I-335; W-43 to I-335; S-44 to I-335; P-45 to I-335; S-46 to I-335; P-47 to I-335; P-48 to I-335; A-49 to I-335; T-50 to I-335; P-51 to I-335; E-52 to I-335; Q-53 to I-335; G-54 to I-335; L-55 to I-335; S-56 to I-335; A-57 to I-335; F-58 to I-335; Y-59 to I-335; L-60 to I-335; S-61 to I-335; Y-62 to I-335; F-63 to I-335; D-64 to I-335; M-65 to I-335; L-66 to I-335; Y-67 to I-335; P-68 to I-335; E-69 to I-335; D-70 to I-335; S-71 to I-335; S-72 to I-335; W-73 to I-335; A-74 to I-335; A-75 to I-335; K-76 to I-335; A-77 to I-335; P-78 to I-335; G-79 to I-335; A-80 to I-335; S-81 to I-335; S-82 to I-335; R-83 to I-335; E-84 to I-335; E-85 to I-335; P-86 to I-335; P-87 to I-335; E-88 to I-335; E-89 to I-335; P-90 to I-335; E-91 to I-335; Q-92 to I-335; C-93 to I-335; P-94 to I-335; V-95 to I-335; I-96 to I-335; D-97 to I-335; S-98 to I-335; Q-99 to I-335; A-100 to I-335; P-101 to I-335; A-102 to I-335; G-103 to I-335; S-104 to I-335; L-105 to I-335; D-106 to I-335; L-107 to I-335; V-108 to I-335; P-109 to I-335; G-110 to I-335; G-111 to I-335; L-112 to I-335; T-113 to I-335; L-114 to I-335; E-115 to I-335; E-116 to I-335; H-117 to I-335; S-118 to I-335; L-119 to I-335; E-120 to I-335; Q-121 to I-335; V-122 to I-335; Q-123 to I-335; S-124 to I-335; M-125 to I-335; V-126 to I-335; V-127 to I-335; G-128 to I-335; E-129 to I-335; V-130 to I-335; L-131 to I-335; K-132 to I-335; D-133 to I-335; I-134 to I-335; E-135 to I-335; T-136 to I-335; A-137 to I-335; C-138 to I-335; K-139 to I-335; L-140 to I-335; L-141 to I-335; N-142 to I-335; I-143 to I-335; T-144 to I-335; A-145 to I-335; D-146 to I-335; P-147 to I-335; M-148 to I-335; D-149 to I-335; W-150 to I-335; S-151 to I-335; P-152 to I-335; S-153 to I-335; N-154 to I-335; V-155 to I-335; Q-156 to I-335; K-157 to I-335; W-158 to I-335; L-159 to I-335; L-160 to I-335; W-161 to I-335; T-162 to I-335; E-163 to I-335; H-164 to I-335; Q-165 to I-335; Y-166 to I-335; R-167 to I-335; L-168 to I-335; P-169 to I-335; P-170 to I-335; M-171 to I-335; G-172 to I-335; K-173 to I-335; A-174 to I-335; F-175 to I-335; Q-176 to I-335; E-177 to I-335; L-178 to I-335; A-179 to I-335; G-180 to I-335; K-181 to I-335; E-182 to I-335; L-183 to I-335; C-184 to I-335; A-185 to I-335; M-186 to I-335; S-187 to I-335; E-188 to I-335; E-189 to I-335; Q-190 to I-335; F-191 to I-335; R-192 to I-335; Q-193 to I-335; R-194 to I-335; S-195 to I-335; P-196 to I-335; L-197 to I-335; G-198 to I-335; G-199 to I-335; D-200 to I-335; V-201 to I-335; L-202 to I-335; H-203 to I-335; A-204 to I-335; H-205 to I-335; L-206 to I-335; D-207 to I-335; 1-208 to I-335; W-209 to I-335; K-210 to I-335; S-211 to I-335; A-212 to I-335; A-213 to I-335; W-214 to I-335; M-215 to I-335; K-216 to I-335; E-217 to I-335; R-218 to I-335; T-219 to I-335; S-220 to I-335; P-221 to I-335; G-222 to I-335; A-223 to I-335; 1-224 to I-335; H-225 to I-335; Y-226 to I-335; C-227 to I-335; A-228 to I-335; S-229 to I-335; T-230 to I-335; S-231 to I-335; E-232 to I-335; E-233 to I-335; S-234 to I-335; W-235 to I-335; T-236 to I-335; D-237 to I-335; S-238 to I-335; E-239 to I-335; V-240 to I-335; D-241 to I-335; S-242 to I-335; S-243 to I-335; C-244 to I-335; S-245 to I-335; G-246 to I-335; Q-247 to I-335; P-248 to I-335; I-249 to I-335; H-250 to I-335; L-251 to I-335; W-252 to I-335; Q-253 to I-335; F-254 to I-335; L-255 to I-335; K-256 to I-335; E-257 to I-335; L-258 to I-335; L-259 to I-335; L-260 to I-335; K-261 to I-335; P-262 to I-335; H-263 to I-335; S-264 to I-335; Y-265 to I-335; G-266 to I-335; R-267 to I-335; F-268 to I-335; I-269 to I-335; R-270 to I-335; W-271 to I-335; L-272 to I-335; N-273 to I-335; K-274 to I-335; E-275 to I-335; K-276 to I-335; G-277 to I-335; I-278 to I-335; F-279 to I-335; K-280 to I-335; 1-281 to I-335; E-282 to I-335; D-283 to I-335; S-284 to I-335; A-285 to I-335; Q-286 to I-335; V-287 to I-335; A-288 to I-335; R-289 to I-335; L-290 to I-335; W-291 to I-335; G-292 to I-335; 1-293 to I-335; R-294 to I-335; K-295 to I-335; N-296 to I-335; R-297 to I-335; P-298 to I-335; A-299 to I-335; M-300 to I-335; N-301 to I-335; Y-302 to I-335; D-303 to I-335; K-304 to I-335; L-305 to I-335; S-306 to I-335; R-307 to I-335; S-308 to I-335; I-309to I-335; R-310to I-335; Q-311 to I-335; Y-312 to I-335; Y-313 to I-335; K-314 to I-335; K-315 to I-335; G-316 to I-335; 1-317 to I-335; 1-318 to I-335; R-319 to I-335; K-320 to I-335; of SEQ ID NO:2.

Moreover, C-terminal deletions of the PDEF polypeptide can also be described by the general formula 1-n, where n is an integer from 15 to 335, where n corresponds to the position of amino acid residue identified in SEQ ID NO:2. Preferably, C-terminal deletions of the PDEF polypeptide of the invention shown as SEQ ID NO:2 include polypeptides comprising the amino acid sequence of residues: M-1 to P-334; M-1 to H-333; M-1 to V-332; M-1 to F-331; M-1 to Q-330; M-1 to Y-329; M-1 to V-328; M-1 to L-327; M-1 to R-326; M-1 to Q-325; M-1 to S-324; M-1 to I-323; M-1 to D-322; M-1 to P-321; M-1 to K-320; M-1 to R-319; M-1 to I-318; M-1 to I-317; M-1 to G-316; M-1 to K-315; M-1 to K-314; M-1 to Y-313; M-1 to Y-312; M-1 to Q-311; M-1 to R-310; M-1 to I-309; M-1 to S-308; M-1 to R-307; M-1 to S-306; M-1 to L-305; M-1 to K-304; M-1 to D-303; M-1 to Y-302; M-1 to N-301; M-1 to M-300; M-1 to A-299; M-1 to P-298; M-1 to R-297; M-1 to N-296; M-1 to K-295; M-1 to R-294; M-1 to I-293; M-1 to G-292; M-1 to W-291; M-1 to L-290; M-1 to R-289; M-1 to A-288; M-1 to V-287; M-1 to Q-286; M-1 to A-285; M-1 to S-284; M-1 to D-283; M-1 to E-282; M-1 to I-281; M-1 to K-280; M-1 to F-279; M-1 to I-278; M-1 to G-277; M-1 to K-276; M-1 to E-275; M-1 to K-274; M-1 to N-273; M-1 to L-272; M-1 to W-271; M-1 to R-270; M-1 to I-269; M-1 to F-268; M-1 to R-267; M-1 to G-266; M-1 to Y-265; M-1 to S-264; M-1 to H-263; M-1 to P-262; M-1 to K-261; M-1 to L-260; M-1 to L-259; M-1 to L-258; M-1 to E-257; M-1 to K-256; M-1 to L-255; M-1 to F-254; M-1 to Q-253;M-1 to W-252; M-1 to L-251; M-1 to H-250; M-1 to I-249; M-1 to P-248; M-1 to Q-247; M-1 to G-246; M-1 to S-245; M-1 to C-244; M-1 to S-243; M-1 to S-242; M-1 to D-241; M-1 to V-240; M-1 to E-239; M-1 to S-238; M-1 to D-237; M-1 to T-236; M-1 to W-235; M-1 to S-234; M-1 to E-233; M-1 to E-232; M-1 to S-231; M-1 to T-230; M-1 to S-229; M-1 to A-228; M-1 to C-227; M-1 to Y-226; M-1 to H-225; M-1 to I-224; M-1 to A-223; M-1 to G-222; M-1 to P-221; M-1 to S-220; M-1 to T-219; M-1 to R-218; M-1 to E-217; M-1 to K-216; M-1 to M-215; M-1 to W-214; M-1 to A-213; M-1 to A-212; M-1 to S-211; M-1 to K-210; M-1 to W-209; M-1 to I-208; M-1 to D-207; M-1 to L-206; M-1 to H-205; M-1 to A-204; M-1 to H-203; M-1 to L-202; M-1 to V-201; M-1 to D-200; M-1 to G-199; M-1 to G-198; M-1 to L-197; M-1 to P-196; M-1 to S-195; M-1 to R-194; M-1 to Q-193; M-1 to R-192; M-1 to F-191; M-1 to Q-190; M-1 to E-189; M-1 to E-188; M-1 to S-187; M-1 to M-186; M-1 to A-185; M-1 to C-184; M-1 to L-183; M-1 to E-182; M-1 to K-181; M-1 to G-180; M-1 to A-179; M-1 to L-178; M-1 to E-177; M-1 to Q-176; M-1 to F-175; M-1 to A-174; M-1 to K-173; M-1 to G-172; M-1 to M-171; M-1 to P-170; M-1 to P-169; M-1 to L-168; M-1 to R-167; M-1 to Y-166; M-1 to Q-165; M-1 to H-164; M-1 to E-163; M-1 to T-162; M-1 to W-161; M-1 to L-160; M-1 to L-159; M-1 to W-158; M-1 to K-157; M-1 to Q-156; M-1 to V-155; M-1 to N-154; M-1 to S-153; M-1 to P-152; M-1 to S-151; M-1 to W-150; M-1 to D-149; M-1 to M-148; M-1 to P-147; M-1 to D-146; M-1 to A-145; M-1 to T-144; M-1 to I-143; M-1 to N-142; M-1 to L-141; M-1 to L-140; M-1 to K-139; M-1 to C-138; M-1 to A-137; M-1 to T-136; M-1 to E-135; M-1 to I-134; M-1 to D-133; M-1 to K-132; M-1 to L-131; M-1 to V-130; M-1 to E-129;M-1 to G-128; M-1 to V-127; M-1 to V-126; M-1 to M-125; M-1 to S-124; M-1 to Q-123; M-1 to V-122; M-1 to Q-121; M-1 to E-120; M-1 to L-119; M-1 to S-118; M-1 to H-117; M-1 to E-116; M-1 to E-115; M-1 to L-114; M-1 to T-113; M-1 to L-112; M-1 to G-111; M-1 to G-110; M-1 to P-109; M-1 to V-108; M-1 to L-107; M-1 to D-106; M-1 to L-105; M-1 to S-104; M-1 to G-103; M-1 to A-102; M-1 to P-101; M-1 to A-100; M-1 to Q-99; M-1 to S-98; M-1 to D-97; M-1 to I-96; M-1 to V-95; M-1 to P-94; M-1 to C-93; M-1 to Q-92; M-1 to E-91; M-1 to P-90; M-1 to E-89; M-1 to E-88; M-1 to P-87; M-1 to P-86; M-1 to E-85; M-1 to E-84; M-1 to R-83; M-1 to S-82; M-1 to S-81; M-1 to A-80; M-1 to G-79; M-1 to P-78; M-1 to A-77; M-1 to K-76; M-1 to A-75; M-1 to A-74; M-1 to W-73; M-1 to S-72; M-1 to S-71; M-1 to D-70; M-1 to E-69; M-1 to P-68; M-1 to Y-67; M-1 to L-66; M-1 to M-65; M-1 to D-64; M-1 to F-63; M-1 to Y-62; M-1 to S-61; M-1 to L-60; M-1 to Y-59; M-1 to F-58; M-1 to A-57; M-1 to S-56; M-1 to L-55; M-1 to G-54; M-1 to Q-53; M-1 to E-52; M-1 to P-51; M-1 to T-50; M-1 to A-49; M-1 to P-48; M-1 to P-47; M-1 to S-46; M-1 to P-45; M-1 to S-44; M-1 to W-43; M-1 to D-42; M-1 to R-41; M-1 to R-40; M-1 to E-39; M-1 to L-38; M-1 to G-37; M-1 to V-36; M-1 to A-35; M-1 to G-34; M-1 to A-33; M-1 to A-32; M-1 to A-31; M-1 to K-30; M-1 to E-29; M-1 to L-28; M-1 to G-27; M-1 to T-26; M-1 to R-25; M-1 to S-24; M-1 to V-23; M-1 to T-22; M-1 to D-21; M-1 to P-20; M-1 to P-19; M-1 to L-18; M-1 to L-17; M-1 to L-16; M-1 to H-15; of SEQ ID NO:2.

The invention also provides polypeptides having one or more amino acids deleted from both the amino and the carboxyl termini, which may be described generally as having residues m-n of SEQ ID NO:2, where n and m are integers as described above. For example, any of the above listed N- or C-terminal deletions can be combined to produce a N- and C-terminal deleted PDEF polypeptide.

Also preferred are PDEF polypeptide and polynucleotide fragments characterized by structural or functional domains. Preferred embodiments of the invention include fragments that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions, substrate binding region, and high antigenic index regions. As set out in the Figures, such preferred regions include Garnier-Robson alpha-regions, beta-regions, turn-regions, and coil-regions, Chou-Fasman alpha-regions, beta-regions, and turn-regions, Kyte-Doolittle hydrophilic regions and hydrophobic regions, Eisenberg alpha and beta amphipathic regions, Karplus-Schulz flexible regions, Emini surface-forming regions, and Jameson-Wolf high antigenic index regions. Polypeptide fragments of SEQ ID NO:2 falling within conserved domains are specifically contemplated by the present invention and shown in the Figures. Moreover, polynucleotide fragments encoding these domains are also contemplated.

Other preferred fragments are biologically active PDEF fragments. Biologically active fragments are those exhibiting activity similar, but not necessarily identical, to an activity of the PDEF polypeptide. The biological activity of the fragments may include an improved desired activity, or a decreased undesirable activity.

However, many polynucleotide sequences, such as EST sequences, are publicly available and accessible through sequence databases. Some of these sequences are related to SEQ ID NO:1 and may have been publicly available prior to conception of the present invention. Preferably, such related polynucleotides are specifically excluded from the scope of the present invention. To list every related sequence would be cumbersome.

Accordingly, preferably excluded from the present invention are one or more polynucleotides comprising a nucleotide sequence described by the general formula of a-b, where a is any integer between 1 to 1878 of SEQ ID NO:1, b is an integer of 15 to 1894, where both a and b correspond to the positions of nucleotide residues shown in SEQ ID NO:1, and where the b is greater than or equal to a+14.

### Epitopes & Antibodies

In the present invention, “epitopes” refer to PDEF polypeptide fragments having antigenic or immunogenic activity in an animal, especially in a human. A preferred embodiment of the present invention relates to a PDEF polypeptide fragment comprising an epitope, as well as the polynucleotide encoding this fragment. A region of a protein molecule to which an antibody can bind is defined as an “antigenic epitope.” In contrast, an “immunogenic epitope” is defined as a part of a protein that elicits an antibody response. (See, for instance, Geysen et al., Proc. Natl. Acad. Sci. USA 81:3998-4002 (1983).)

Fragments which function as epitopes may be produced by any conventional means. (See, e.g., Houghten, R. A., Proc. Natl. Acad. Sci. USA 82:5131-5135 (1985) further described in U.S. Pat. No. 4,631,211.)

In the present invention, antigenic epitopes preferably contain a sequence of at least seven, more preferably at least nine, and most preferably between about 15 to about 30 amino acids. Antigenic epitopes are useful to raise antibodies, including monoclonal antibodies, that specifically bind the epitope. (See, for instance, Wilson et al., Cell 37:767-778 (1984); Sutcliffe, J. G. et al., Science 219:660-666 (1983).)

Similarly, immunogenic epitopes can be used to induce antibodies according to methods well known in the art. (See, for instance, Sutcliffe et al., supra; Wilson et al., supra; Chow, M. et al., Proc. Natl. Acad. Sci. USA 82:910-914; and Bittle, F. J. et al., J. Gen. Virol. 66:2347-2354(1985).) A preferred immunogenic epitope includes the soluble protein. The immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting.)

Using DNAstar analysis, SEQ ID NO:2 was found antigenic at amino acids: Asp21-Glu29, Leu38-Ser46, Ser46-Gly54, Leu66-Ala74, Ala75-Arg83, Glu84-Gln92, Val130-Cys138, Asp146-Asn154, Gln165-Lys173, Leu178-Met186, Arg192-Asp200, Met215-Gly222, Ser229-Asp237, Glu239-Gln247, Ser234-Ser242, Leu272-Lys280, Phe279-Val287, Gly292-Met300, Asn301-Ile309, Ile317-Gln325. Thus, these regions could be used as epitopes to produce antibodies against the protein encoded by SEQ ID No:1.

As used herein, the term “antibody” (Ab) or “monoclonal antibody” (Mab) is meant to include intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody. (Wahl et al., J. Nucl. Med. 24:316-325 (1983).) Thus, these fragments are preferred, as well as the products of a FAB or other immunoglobulin expression library. Moreover, antibodies of the present invention include chimeric, single chain, and humanized antibodies.

### Fusion Proteins

Any PDEF polypeptide can be used to generate fusion proteins. For example, the PDEF polypeptide, when fused to a second protein, can be used as an antigenic tag. Antibodies raised against the PDEF polypeptide can be used to indirectly detect the second protein by binding to the PDEF. Moreover, because secreted proteins target cellular locations based on trafficking signals, the PDEF polypeptides can be used as a targeting molecule once fused to other proteins.

Examples of domains that can be fused to PDEF polypeptides include not only heterologous signal sequences, but also other heterologous functional regions. The fusion does not necessarily need to be direct, but may occur through linker sequences.

Moreover, fusion proteins may also be engineered to improve characteristics of the PDEF polypeptide. For instance, a region of additional amino acids, particularly charged amino acids, may be added to the N-terminus of the PDEF polypeptide to improve stability and persistence during purification from the host cell or subsequent handling and storage. Also, peptide moieties may be added to the PDEF polypeptide to facilitate purification. Such regions may be removed prior to final preparation of the PDEF polypeptide. The addition of peptide moieties to facilitate handling of polypeptides are familiar and routine techniques in the art.

Moreover, PDEF polypeptides, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. (EP A 394,827; Traunecker et al., Nature 331:84-86 (1988).) Fusion proteins having disulfide-linked dimeric structures (due to the IgG) can also be more efficient in binding and neutralizing other molecules, than the monomeric secreted protein or protein fragment alone. (Fountoulakis et al., J. Biochem. 270:3958-3964 (1995).)

Similarly, EP-A-O 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, D. Bennett et al., J. Molecular Recognition 8:52-58 (1995); K. Johanson et al., J. Biol. Chem. 270:9459-9471 (1995).)

Moreover, the PDEF polypeptides can be fused to marker sequences, such as a peptide which facilitates purification of PDEF. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Another peptide tag useful for purification, the “HA” tag, corresponds to an epitope derived from the influenza hemagglutinin protein. (Wilson et al., Cell 37:767 (1984).)

Thus, any of these above fusions can be engineered using the PDEF polynucleotides or the polypeptides.

### Vectors, Host Cells, and Protein Production

The present invention also relates to vectors containing the PDEF polynucleotide, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

PDEF polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The PDEF polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that PDEF polypeptides may in fact be expressed by a host cell lacking a recombinant vector.

PDEF polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“BPLC”) is employed for purification.

PDEF polypeptides can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the PDEF polypeptides may be glycosylated or may be non-glycosylated. In addition, PDEF polypeptides may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

### Uses of the PDEF Polynucleotides

The PDEF polynucleotides identified herein can be used in numerous ways as reagents. The following description should be considered exemplary and utilizes known techniques.

There exists an ongoing need to identify new chromosome markers, since few chromosome marking reagents, based on actual sequence data (repeat polymorphisms), are presently available. This gene maps to 6p21.3. Thus, PDEF polynucleotides can be used in linkage analysis as a marker for chromosome 6p21.3.

Briefly, sequences can be mapped to chromosomes by preparing PCR primers (preferably 15-25 bp) from the sequences shown in SEQ ID NO:1. Primers can be selected using computer analysis so that primers do not span more than one predicted exon in the genomic DNA. These primers are then used for PCR screening of somatic cell hybrids containing individual human chromosomes. Only those hybrids containing the human PDEF gene corresponding to the SEQ ID NO:1 will yield an amplified fragment.

Similarly, somatic hybrids provide a rapid method of PCR mapping the polynucleotides to particular chromosomes. Three or more clones can be assigned per day using a single thermal cycler. Moreover, sublocalization of the PDEF polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the PDEF polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred. For a review of this technique, see Verma et al., “Human Chromosomes: a Manual of Basic Techniques,” Pergamon Press, New York (1988).

For chromosome mapping, the PDEF polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. (Disease mapping data are found, for example, in V. McKusick, Mendelian Inheritance in Man (available on line through Johns Hopkins University Welch Medical Library).) Assuming 1 megabase mapping resolution and one gene per 20 kb, a cDNA precisely localized to a chromosomal region associated with the disease could be one of 50-500 potential causative genes.

Thus, once coinheritance is established, differences in the PDEF polynucleotide and the corresponding gene between affected and unaffected individuals can be examined. First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the PDEF polypeptide and the corresponding gene from several normal individuals is required to distinguish the mutation from a polymorphism. If a new polymorphism is identified, this polymorphic polypeptide can be used for further linkage analysis.

Furthermore, increased or decreased expression of the gene in affected individuals as compared to unaffected individuals can be assessed using PDEF polynucleotides. Any of these alterations (altered expression, chromosomal rearrangement, or mutation) can be used as a diagnostic or prognostic marker.

In addition to the foregoing, a PDEF polynucleotide can be used to control gene expression through triple helix formation or antisense DNA or RNA. Both methods rely on binding of the polynucleotide to DNA or RNA. For these techniques, preferred polynucleotides are usually 20 to 40 bases in length and complementary to either the region of the gene involved in transcription (triple helix—see Lee et al., Nucl. Acids Res. 6:3073 (1979); Cooney et al., Science 241:456 (1988); and Dervan et al., Science 251:1360 (1991)) or to the mRNA itself (antisense—Okano, J. Neurochem. 56:560 (1991); Oligodeoxy-nucleotides as Antisense Inhibitors of Gene Expression, CRC Press, Boca Raton, Fla.(1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat disease.

PDEF polynucleotides are also useful in gene therapy. One goal of gene therapy is to insert a normal gene into an organism having a defective gene, in an effort to correct the genetic defect. PDEF offers a means of targeting such genetic defects in a highly accurate manner. Another goal is to insert a new gene that was not present in the host genome, thereby producing a new trait in the host cell. Yet another goal is to make dominant-negative mutants of PDEF to inhibit PDEF function in disease.

The PDEF polynucleotides are also useful for identifying individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The PDEF polynucleotides can be used as additional DNA markers for RFLP.

The PDEF polynucleotides can also be used as an alternative to RFLP, by determining the actual base-by-base DNA sequence of selected portions of an individual's genome. These sequences can be used to prepare PCR primers for amplifying and isolating such selected DNA, which can then be sequenced. Using this technique, individuals can be identified because each individual will have a unique set of DNA sequences. Once an unique ID database is established for an individual, positive identification of that individual, living or dead, can be made from extremely small tissue samples.

Forensic biology also benefits from using DNA-based identification techniques as disclosed herein. DNA sequences taken from very small biological samples such as tissues, e.g., hair or skin, or body fluids, e.g., blood, saliva, semen, etc., can be amplified using PCR. In one prior art technique, gene sequences amplified from polymorphic loci, such as DQa class II HLA gene, are used in forensic biology to identify individuals. (Erlich, H., PCR Technology, Freeman and Co. (1992).) Once these specific polymorphic loci are amplified, they are digested with one or more restriction enzymes, yielding an identifying set of bands on a Southern blot probed with DNA corresponding to the DQa class II HLA gene. Similarly, PDEF polynucleotides can be used as polymorphic markers for forensic purposes.

There is also a need for reagents capable of identifying the source of a particular tissue. Such need arises, for example, in forensics when presented with tissue of unknown origin. Appropriate reagents can comprise, for example, DNA probes or primers specific to particular tissue prepared from PDEF sequences. Panels of such reagents can identify tissue by species and/or by organ type. In a similar fashion, these reagents can be used to screen tissue cultures for contamination.

Because PDEF was found to be expressed in prostate epithelium, and to a lesser extent in salivary gland, trachea, ovary, and mammary, PDEF polynucleotides are useful as hybridization probes for differential identification of the tissue(s) or cell type(s) present in a biological sample. Similarly, polypeptides and antibodies directed to PDEF polypeptides are useful to provide immunological probes for differential identification of the tissue(s) or cell type(s). In addition, for a number of disorders of the above tissues or cells, particularly of the reproductive system, significantly higher or lower levels of PDEF gene expression, or abnormal PDEF expression in tissues which normally do not express PDEF, may be detected in certain tissues (e.g., cancerous and wounded tissues) or bodily fluids (e.g., serum, plasma, urine, synovial fluid or spinal fluid) taken from an individual having such a disorder, relative to a “standard” PDEF gene expression level, i.e., the PDEF expression level in healthy tissue from an individual not having the reproductive system disorder.

Thus, the invention provides a diagnostic method of a disorder, which involves: (a) assaying PDEF gene expression level in cells or body fluid of an individual; (b) comparing the PDEF gene expression level with a standard PDEF gene expression level, whereby an increase or decrease in the assayed PDEF gene expression level compared to the standard expression level is indicative of disorder in the reproductive system, or other tissues.

In the very least, the PDEF polynucleotides can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

### Uses of PDEF Polypeptides

PDEF polypeptides can be used in numerous ways. The following description should be considered exemplary and utilizes known techniques.

PDEF polypeptides can be used to assay protein levels in a biological sample using antibody-based techniques. For example, protein expression in tissues can be studied with classical immunohistological methods. (Jalkanen, M., et al., J. Cell. Biol. 101:976-985 (1985); Jalkanen, M., et al., J. Cell . Biol. 105:3087-3096 (1987).) Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (125I, 121I), carbon (14C), sulfur (35S), tritium (3H), indium (112In), and technetium (99mTc), and fluorescent labels, such as fluorescein and rhodamine, and biotin.

In addition to assaying protein levels in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which maybe incorporated into the antibody by labeling of nutrients for the relevant hybridoma.

A protein-specific antibody or antibody fragment which has been labeled with an appropriate detectable imaging moiety, such as a radioisotope (for example, 131I, 112In, 99mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).)

Thus, the invention provides a diagnostic method of a disorder, which involves (a) assaying the expression of PDEF polypeptide in cells or body fluid of an individual; (b) comparing the level of gene expression with a standard gene expression level, whereby an increase or decrease in the assayed PDEF polypeptide gene expression level compared to the standard expression level is indicative of a disorder.

Moreover, PDEF polypeptides can be used to treat disease. For example, patients can be administered PDEF polypeptides in an effort to replace absent or decreased levels of the PDEF polypeptide (e.g., insulin), to supplement absent or decreased levels of a different polypeptide (e.g., hemoglobin S for hemoglobin B), to inhibit the activity of a polypeptide (e.g., an oncogene), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth). In addition, the PDEF gene promoter and/or enhancer element can be used in gene therapy applications for treating prostate-specific disorders, particularly prostate cancer or tumors.

Similarly, antibodies directed to PDEF polypeptides can also be used to treat disease. For example, administration of an antibody directed to a PDEF polypeptide can bind and reduce overproduction of the polypeptide. Similarly, administration of an antibody can activate the polypeptide, such as by binding to a polypeptide bound to a membrane (receptor).

At the very least, the PDEF polypeptides can be used as molecular weight markers on SDS-PAGE gels or on molecular sieve gel filtration columns using methods well known to those of skill in the art. PDEF polypeptides can also be used to raise antibodies, which in turn are used to measure protein expression from a recombinant cell, as a way of assessing transformation of the host cell. Moreover, PDEF polypeptides can be used to test the following biological activities.

### Biological Activities of PDEF

PDEF polynucleotides and polypeptides can be used in assays to test for one or more biological activities. If PDEF polynucleotides and polypeptides do exhibit activity in a particular assay, it is likely that PDEF may be involved in the diseases associated with the biological activity. Therefore, PDEF could be used to treat the associated disease.

For example, the mechanisms involved in the development and maintenance of prostatic tissue are poorly understood. Although it has been recognized that normal development and continued expression in adults of the secondary sexual phenotype is androgen-dependent, there is relatively little known about the genes on which androgens act or the downstream pathways that lead to development of differentiated tissue. As with prostate development, the fundamental mechanisms underlying prostate cancer also remain obscure. However, androgen regulation and the loss thereof is known to play a critical role. In both developing and mature prostate, the maintenance of prostate-specific cellular functions requires continuous stimulation by androgens; in prostate-specific cancer tissue, the reciprocal loss of this cellular differentiation, which occurs during progression of the disease, is largely concomitant with a loss of androgen responsiveness by prostatic cells. Identifying the genes involved in either of these largely opposing processes, will likely lead to a greater understanding of the fundamental mechanisms involved.

PDEF polynucleotides and polypeptides, but not other Ets factors, have been shown to activate the prostate-specific antigen (PSA) gene through direct enhancement of PSA promoter transcription. The PSA gene encodes a secreted protein which has significant diagnostic utility as a clinical marker for prostate cancer. In contrast, none of the other Ets factors have been shown to significantly effect PSA promoter activity. Thus, the fact that the PSA gene can be activated by PDEF demonstrates the utility of PDEF as a positive regulator of PSA gene transcription and implies that PSA might indeed be a relevant prostate-specific target for PDEF.

Moreover, a characteristic feature of all Ets factors is their ability to interact with other transcription factors. Following this reasoning, PDEF may interact with factors that bind to regulatory elements within the PSA promoter, which maybe an important mechanism of transcriptional control. In addition, PDEF specifically binds to the DNA binding domain of the androgen receptor and cooperates with androgen in activating the PSA promoter. Such cooperativity has been shown to derive from direct physical interaction between PDEF and the androgen receptor.

Strong evidence for epithelial cell specificity is provided by both RT/PCR analysis of individual cell types as well as in situ hybridization. PDEF is primarily expressed in the epithelial cells of the prostate and to lower extent in several additional hormone regulated glandular tissues. In the prostate, PDEF expression is restricted to the luminal epithelial cells which are the exact cells that secrete the prostate cancer marker PSA. It has been demonstrated that the PSA gene is an apparent target for PDEF and that PDEF interacts with the androgen receptor and synergizes in the presence of androgen to induce PSA gene transcription. In correlation with the in situ hybridization results (Example 3 of present invention), PDEF expression is lower in unstimulated prostate cancer cells and becomes induced by androgen which also upregulates PSA. The expression pattern of PDEF suggests that PDEF regulates a distinct group of prostate-specific genes. Only a few epithelial-specific genes have been previously shown to be regulated by Ets factors and none of them is prostate-specific.

Therefore, due to the novel activities of the PDEF polynucleotides and polypeptides, PDEF may be used as a prostate-specific tumor marker for the diagnosis of prostate cancer. Considering the physical interaction between PDEF and the androgen receptor, PDEF may also serve as a potentially important therapeutic for prostate cancer.

Immune Activity

PDEF polypeptides or polynucleotides may be useful in treating deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, PDEF polynucleotides or polypeptides can be used as a marker or detector of a particular immune system disease or disorder.

PDEF polynucleotides or polypeptides may be useful in treating or detecting deficiencies or disorders of hematopoietic cells. PDEF polypeptides or polynucleotides could be used to increase differentiation and proliferation of hematopoietic cells, including the pluripotent stem cells, in an effort to treat those disorders associated with a decrease in certain (or many) types hematopoietic cells. Examples of immunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, PDEF polypeptides or polynucleotides can also be used to modulate hemostatic (the stopping of bleeding) or thrombolytic activity (clot formation). For example, by increasing hemostatic or thrombolytic activity, PDEF polynucleotides or polypeptides could be used to treat blood coagulation disorders (e.g., afibrinogenemia, factor deficiencies), blood platelet disorders (e.g. thrombocytopenia), or wounds resulting from trauma, surgery, or other causes. Alternatively, PDEF polynucleotides or polypeptides that can decrease hemostatic or thrombolytic activity could be used to inhibit or dissolve clotting, important in the treatment of heart attacks (infarction), strokes, or scarring.

PDEF polynucleotides or polypeptides may also be useful in treating or detecting autoimmune disorders. Many autoimmune disorders result from inappropriate recognition of self as foreign material by immune cells. This inappropriate recognition results in an immune response leading to the destruction of the host tissue. Therefore, the administration of PDEF polypeptides or polynucleotides that can inhibit an immune response, particularly the proliferation, differentiation, or chemotaxis of T-cells, may be an effective therapy in preventing autoimmune disorders.

Examples of autoimmune disorders that can be treated or detected by PDEF include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Prostate Cancer, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated by PDEF polypeptides or polynucleotides. Moreover, PDEF can be used to treat anaphylaxis, hypersensitivity to an antigenic molecule, or blood group incompatibility.

PDEF polynucleotides or polypeptides may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of PDEF polypeptides or polynucleotides that inhibits an immune response, particularly the proliferation, differentiation, or chemotaxis of T-cells, may be an effective therapy in preventing organ rejection or GVHD.

Similarly, PDEF polypeptides or polynucleotides may also be used to modulate inflammation. For example, PDEF polypeptides or polynucleotides may inhibit the proliferation and differentiation of cells involved in an inflammatory response. These molecules can be used to treat inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.)

Hyperproliferative Disorders

PDEF polypeptides or polynucleotides can be used to treat or detect hyperproliferative disorders, including neoplasms. PDEF polypeptides or polynucleotides may inhibit the proliferation of the disorder through direct or indirect interactions. Alternatively, PDEF polypeptides or polynucleotides may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated. This immune response maybe increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by PDEF polynucleotides or polypeptides include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, urogenital, and prostate. Moreover, specific types of cancers that can be treated or detected by PDEF include: non-Hodgkin's lymphoma, follicle center lymphoma, ovarian cancer, multiple myeloma, cervical cancer, acute T-lymphoblastic leukemia, endometrial cancer, germ cell tumor, glioma, acute leukemias, CML, and melanoma.

Similarly, other hyperproliferative disorders can also be treated or detected by PDEF polynucleotides or polypeptides. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Infectious Disease

PDEF polypeptides or polynucleotides can be used to treat or detect infectious agents. For example, by increasing the immune response, particularly increasing the proliferation and differentiation of B and/or T cells, infectious diseases may be treated. The immune response may be increased by either enhancing an existing immune response, or by initiating a new immune response. Alternatively, PDEF polypeptides or polynucleotides may also directly inhibit the infectious agent, without necessarily eliciting an immune response.

Viruses are one example of an infectious agent that can cause disease or symptoms that can be treated or detected by PDEF polynucleotides or polypeptides. Examples of viruses, include, but are not limited to the following DNA and RNA viral families: Arbovirus, Adenoviridae, Arenaviridae, Arterivirus, Birnaviridae, Bunyaviridae, Caliciviridae, Circoviridae, Coronaviridae, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picornaviridae, Poxviridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. PDEF polypeptides or polynucleotides can be used to treat or detect any of these symptoms or diseases.

Similarly, bacterial or fungal agents that can cause disease or symptoms and that can be treated or detected by PDEF polynucleotides or polypeptides include, but not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. PDEF polypeptides or polynucleotides can be used to treat or detect any of these symptoms or diseases.

Moreover, parasitic agents causing disease or symptoms that can be treated or detected by PDEF polynucleotides or polypeptides include, but not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. PDEF polypeptides or polynucleotides can be used to treat or detect any of these symptoms or diseases.

Preferably, treatment using PDEF polypeptides or polynucleotides could either be by administering an effective amount of PDEF polypeptide to the patient, or by removing cells from the patient, supplying the cells with PDEF polynucleotide, and returning the engineered cells to the patient (ex vivo therapy). Moreover, the PDEF polypeptide or polynucleotide can be used as an antigen in a vaccine to raise an immune response against infectious disease.

Regeneration

PDEF polynucleotides or polypeptides can be used to alter the expression of genes that differentiate, proliferate, and attract cells, leading to the regeneration of tissues. (See, Science 276:59-87 (1997).) The regeneration of tissues could be used to repair, replace, or protect tissue damaged by congenital defects, trauma (wounds, bums, incisions, or ulcers), age, disease (e.g. osteoporosis, osteoarthritis, periodontal disease, liver failure), surgery, including cosmetic plastic surgery, fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vascular (including vascular endothelium), nervous, hematopoietic, and skeletal (bone, cartilage, tendon, and ligament) tissue. Preferably, regeneration occurs without or decreased scarring. Regeneration also may include angiogenesis.

Moreover, PDEF polynucleotides or polypeptides may increase regeneration of tissues difficult to heal. For example, increased tendon/ligament regeneration would quicken recovery time after damage. PDEF polynucleotides or polypeptides of the present invention could also be used prophylactically in an effort to avoid damage. Specific diseases that could be treated include of tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using PDEF polynucleotides or polypeptides to proliferate and differentiate nerve cells. Diseases that could be treated using this method include central and peripheral nervous system diseases, neuropathies, or mechanical and traumatic disorders (e.g., spinal cord disorders, head trauma, cerebrovascular disease, and stoke). Specifically, diseases associated with peripheral nerve injuries, peripheral neuropathy (e.g., resulting from chemotherapy or other medical therapies), localized neuropathies, and central nervous system diseases (e.g., Alzheimer's disease, Parkinson's disease, Huntington's disease, amyotrophic lateral sclerosis, and Shy-Drager syndrome), could all be treated using the PDEF polynucleotides or polypeptides.

Chemotaxis

PDEF polynucleotides or polypeptides may alter the expression of genes that have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

PDEF polynucleotides or polypeptides may increase chemotaxic activity of particular cells. These chemotactic molecules can then be used to treat inflammation, infection, hyperproliferative disorders, or any immune system disorder by increasing the number of cells targeted to a particular location in the body. For example, chemotaxic molecules can be used to treat wounds and other trauma to tissues by attracting immune cells to the injured location. As a chemotactic molecule, PDEF could also attract fibroblasts, which can be used to treat wounds.

It is also contemplated that PDEF polynucleotides or polypeptides may inhibit chemotactic activity. These molecules could also be used to treat disorders. Thus, PDEF polynucleotides or polypeptides could be used as an inhibitor of chemotaxis.

Binding Activity

PDEF polypeptides may be used to screen for proteins that bind to PDEF or for proteins to which PDEF binds. The binding of PDEF and the molecule may activate (agonist), increase, inhibit (antagonist), or decrease activity of the PDEF or the molecule bound. Examples of such molecules include antibodies, oligonucleotides, proteins (e.g., receptors),or small molecules.

Preferably, the molecule is closely related to the natural ligand of PDEF, e.g., a fragment of the ligand, or a natural substrate, a ligand, a structural or functional mimetic. (See, Coligan et al., Current Protocols in Immunology 1(2):Chapter 5 (1991).) Similarly, the molecule can be closely related to the natural receptor to which PDEF binds, or at least, a fragment of the receptor capable of being bound by PDEF (e.g., active site). In either case, the molecule can be rationally designed using known techniques.

Preferably, the screening for these molecules involves producing appropriate cells which express PDEF, either as a secreted protein or on the cell membrane. Preferred cells include cells from mammals, yeast, Drosophila, or E. coli. Cells expressing PDEF (or cell membrane containing the expressed polypeptide) are then preferably contacted with a test compound potentially containing the molecule to observe binding, stimulation, or inhibition of activity of either PDEF or the molecule.

The assay may simply test binding of a candidate compound to PDEF, wherein binding is detected by a label, or in an assay involving competition with a labeled competitor. Further, the assay may test whether the candidate compound results in a signal generated by binding to PDEF.

Alternatively, the assay can be carried out using cell-free preparations, polypeptide/molecule affixed to a solid support, chemical libraries, or natural product mixtures. The assay may also simply comprise the steps of mixing a candidate compound with a solution containing PDEF, measuring PDEF/molecule activity or binding, and comparing the PDEF/molecule activity or binding to a standard.

Preferably, an ELISA assay can measure PDEF level or activity in a sample (e.g., biological sample) using a monoclonal or polyclonal antibody. The antibody can measure PDEF level or activity by either binding, directly or indirectly, to PDEF or by competing with PDEF for a substrate.

All of these above assays can be used as diagnostic or prognostic markers. The molecules discovered using these assays can be used to treat disease or to bring about a particular result in a patient (e.g., blood vessel growth) by activating or inhibiting the PDEF/molecule. Moreover, the assays can discover agents which may inhibit or enhance the production of PDEF from suitably manipulated cells or tissues.

Therefore, the invention includes a method of identifying compounds which bind to PDEF comprising the steps of: (a) incubating a candidate binding compound with PDEF; and (b) determining if binding has occurred. Moreover, the invention includes a method of identifying agonists/antagonists comprising the steps of: (a) incubating a candidate compound with PDEF, (b) assaying a biological activity, and (b) determining if a biological activity of PDEF has been altered.

Other Activities

PDEF polypeptides or polynucleotides may also alter the expression of genes that increase or decrease the differentiation or proliferation of embryonic stem cells, besides, as discussed above, hematopoietic lineage.

PDEF polypeptides or polynucleotides may also be used to modulate mammalian characteristics, such as body height, weight, hair color, eye color, skin, percentage of adipose tissue, pigmentation, size, and shape (e.g., cosmetic surgery). Similarly, PDEF polypeptides or polynucleotides may be used to modulate mammalian metabolism affecting catabolism, anabolism, processing, utilization, and storage of energy.

PDEF polypeptides or polynucleotides may be used to change a mammal's mental state or physical state by influencing biorhythms, circadian rhythms, depression (including depressive disorders), tendency for violence, tolerance for pain, reproductive capabilities (preferably by Activin or Inhibin-like activity), hormonal or endocrine levels, appetite, libido, memory, stress, or other cognitive qualities.

PDEF polypeptides or polynucleotides may also be used as a food additive or preservative, such as to increase or decrease storage capabilities, fat content, lipid, protein, carbohydrate, vitamins, minerals, cofactors or other nutritional components.

PDEF polypeptides or polynucleotides may be used to identify novel target genes by cDNA array transcriptional profiling.

PDEF polypeptides or polynucleotides may be used to identify DNA sequences which are targets for PDEF.

The PDEF polypeptides or polynucleotides may be used to identify the prostate-specific promoter as a tool for prostate-specific gene therapy. For example, the promoter of PDEF maybe fused to a coding sequence to obtain prostate specific expresssion of the coding sequence.

Having generally described the invention, the same will be more readily understood by reference to the following examples, which are provided by way of illustration and are not intended as limiting.

### EXAMPLES

### Example 1

### Isolation of the PDEF CDNA Clone From the Deposited Sample

The cDNA for PDEF is inserted into the EcoRi multiple cloning site pCI. (Promega.) pCI contains an ampicillin resistance gene and may be transformed into E. coli strain DH10B, available from Life Technologies. (See, for instance, Gruber, C. E., et al., Focus 15:59- (1993).)

Two approaches can be used to isolate PDEF from the deposited sample. First, a specific polynucleotide of SEQ ID NO:1 with 30-40 nucleotides is synthesized using an Applied Biosystems DNA synthesizer according to the sequence reported. The oligonucleotide is labeled, for instance, with ³²P-γ-ATP using T4 polynucleotide kinase and purified according to routine methods. (E.g., Maniatis et al., Molecular Cloning: A Laboratory Manual, Cold Spring Harbor Press, Cold Spring, N.Y. (1982).) The plasmid mixture is transformed into a suitable host (such as XL-1 Blue (Stratagene)) using techniques known to those of skill in the art, such as those provided by the vector supplier or in related publications or patents. The transformants are plated on 1.5% agar plates (containing the appropriate selection agent, e.g., ampicillin) to a density of about 150 transformants (colonies) per plate. These plates are screened using Nylon membranes according to routine methods for bacterial colony screening (e.g., Sambrook et al., Molecular Cloning: A Laboratory Manual, 2nd Edit., (1989), Cold Spring Harbor Laboratory Press, pages 1.93 to 1.104), or other techniques known to those of skill in the art.

Alternatively, two primers of 17-20 nucleotides derived from both ends of the SEQ ID NO:1 (i.e., within the region of SEQ ID NO:1 bounded by the 5′ NT and the 3′ NT of the clone) are synthesized and used to amplify the PDEF cDNA using the deposited cDNA plasmid as a template. The polymerase chain reaction is carried out under routine conditions, for instance, in 25 μl of reaction mixture with 0.5 ug of the above cDNA template. A convenient reaction mixture is 1.5-5 mM MgCl₂, 0.01% (w/v) gelatin, 20 μM each of dATP, dCTP, dGTP, dTTP, 25 pmol of each primer and 0.25 Unit of Taq polymerase. Thirty five cycles of PCR (denaturation at 94° C. for 1 min; annealing at 55° C. for 1 min; elongation at 72° C. for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subcloning and sequencing the DNA product.

Several methods are available for the identification of the 5′ or 3′ non-coding portions of the PDEF gene which may not be present in the deposited clone. These methods include but are not limited to, filter probing, clone enrichment using specific probes, and protocols similar or identical to 5′ and 3′ “RACE” protocols which are well known in the art. For instance, a method similar to 5′ RACE is available for generating the missing 5′ end of a desired full-length transcript. (Fromont-Racine et al., Nucleic Acids Res. 21(7):1683-1684 (1993).)

Briefly, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the PDEF gene of interest is used to PCR amplify the 5′ portion of the PDEF full-length gene. This amplified product may then be sequenced and used to generate the full length gene.

This above method starts with total RNA isolated from the desired source, although poly-A+ RNA can be used. The RNA preparation can then be treated with phosphatase if necessary to eliminate 5′ phosphate groups on degraded or damaged RNA which may interfere with the later RNA ligase step. The phosphatase should then be inactivated and the RNA treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the PDEF gene.

### Example 2

### Isolation of PDEF Genomic Clones

A human genomic P1 library (Genomic Systems, Inc.) is screened by PCR using primers selected for the cDNA sequence corresponding to SEQ ID NO:1, according to the method described in Example 1. (See also, Sambrook.)

### Example 3

### Tissue Distribution of PDEF Polypeptides

Tissue distribution of mRNA expression of PDEF is determined using protocols for Northern blot analysis, described by, among others, Sanbrook et al. For example, a PDEF probe produced by the method described in Example 1 is labeled with P³² using the RediPrime™ DNA labeling system (Amersham Life Science), according to manufacturer's instructions. After labeling, the probe is purified using CHROMA SPIN-100™ column (Clontech Laboratories, Inc.), according to manufacturer's protocol number PT1200-1. The purified labeled probe is then used to examine various human tissues for mRNA expression.

Multiple Tissue Northern (MTN) blots containing various human tissues (H) or human immune system tissues (IM) (Clontech) are examined with the labeled probe using ExpressHyb™ hybridization solution (Clontech) according to manufacturer's protocol number PT1190-1. Following hybridization and washing, the blots are mounted and exposed to film at −70° C. overnight, and the films developed according to standard procedures.

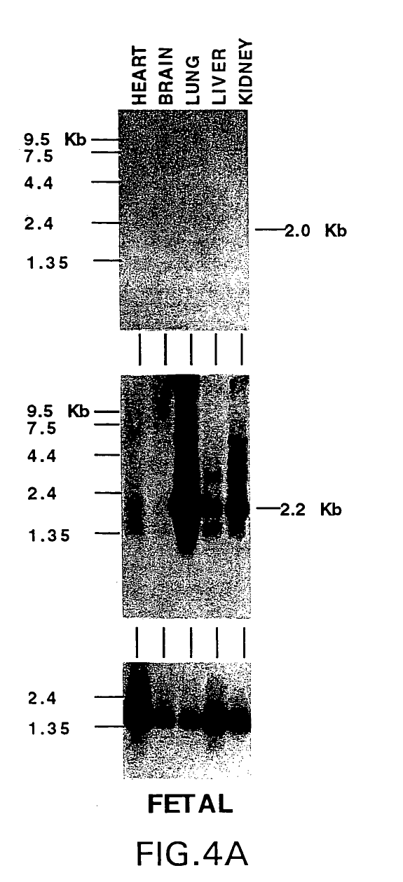

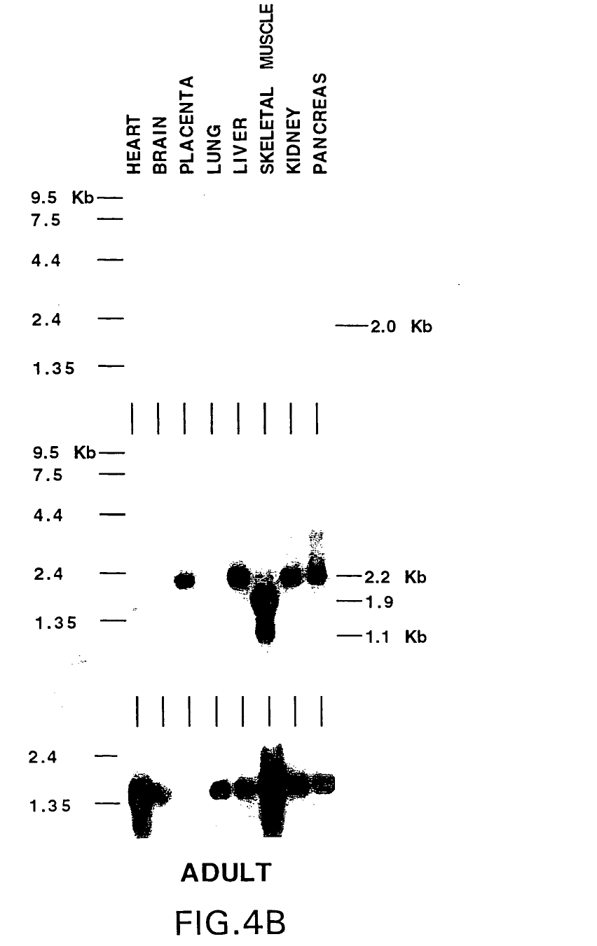

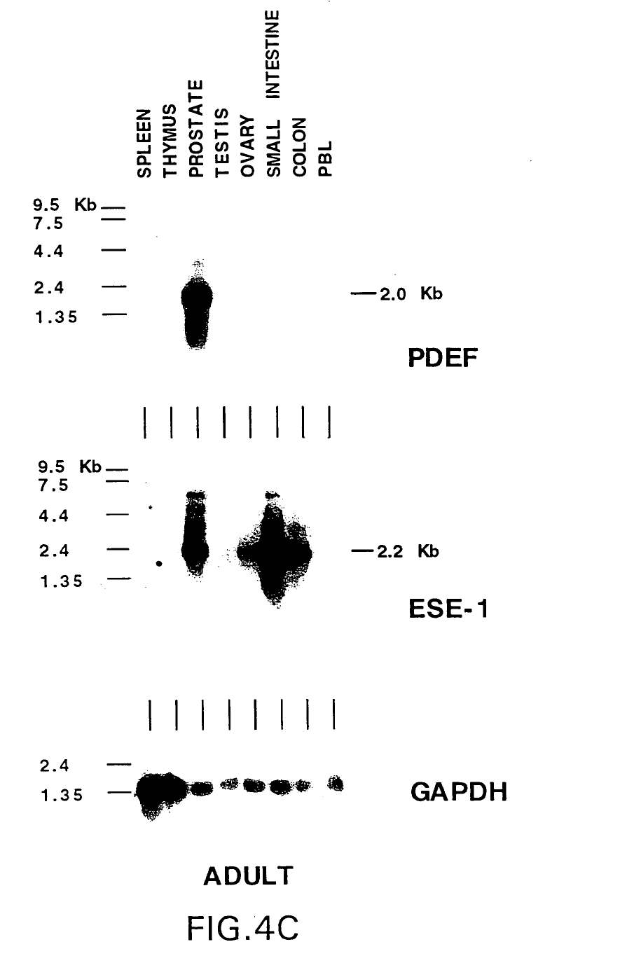

For example, poly(A)+mRNAs derived from various human tissues were analyzed by Northern blot hybridization and dot blot hybridization using PDEF cDNA as a probe. (See FIG. 4). The Northern blots were rehybridized with a GAPDH probe to control for RNA quality and quantity. Conditions for hybridization included: Northern blots and dot blots containing poly(A)+ selected mRNA derived from different human tissues (Clontech) were hybridized with random prime labeled PDEF, ESE-1, and GAPDH cDNA in QuickHyb solution (Stratagene) and washed at 50° C. with 0.2×SSC, 0.2% SDS.

The results indicate the presence of one predominant PDEF transcript of approximately 2.0 kb. PDEF is highly and almost exclusively expressed in prostate, a strikingly different expression pattern from any member of the Ets family. In human fetal tissues, no expression was detected in any tissue by Northern blot hybridization. In adult tissues, prostate expressed the highest levels of PDEF. Low levels of PDEF transcripts were only found in ovary, trachea, and stomach, whereas no expression was detected in all the other tissues.

These results suggest that PDEF is expressed in a very restricted set of tissues and, therefore, has a very specialized fiction. To compare expression of PDEF to expression of another Ets factor, the Northern blots were rehybridized with a cDNA probe for ESE-1. In fetal tissues ESE-1 was expressed in lung, liver and kidney. In adult tissues highest levels of ESE-1 were found in small intestine, prostate, colon, pancreas, kidney, liver, and placenta. Thus, the expression pattern of ESE-1 is strikingly different from PDEF with ESE-1 expression especially high in the gastrointestinal system, fetal lung, and several other epithelial cell tissues.

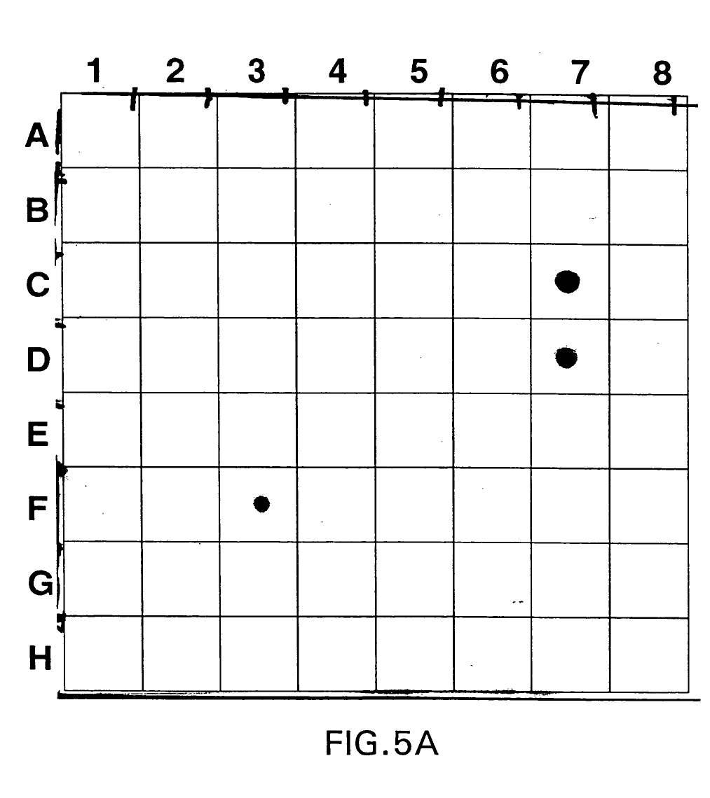

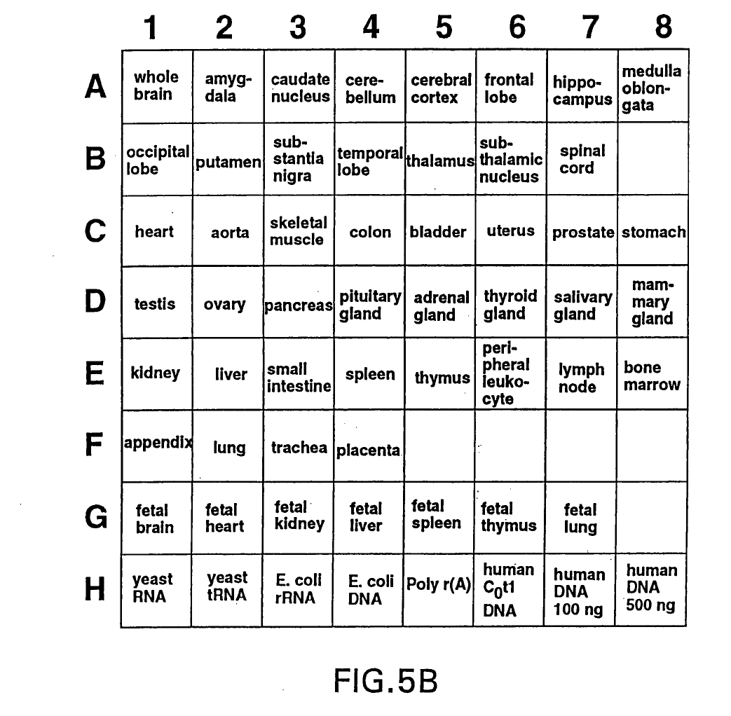

Moreover, dot blot analysis of a whole panel of human RNAs confirmed the highly restricted expression pattern of PDEF demonstrating that PDEF is highly expressed in prostate, to a lower extent in salivary gland and trachea, and weakly in mammary gland, stomach, and lung, (See FIG. 5). RNA blots (CLONTECH) were hybridized in QuickHyb solution (Stratagene), and washed at 50° C. with 0.2×SSC, 0.2% SDS.

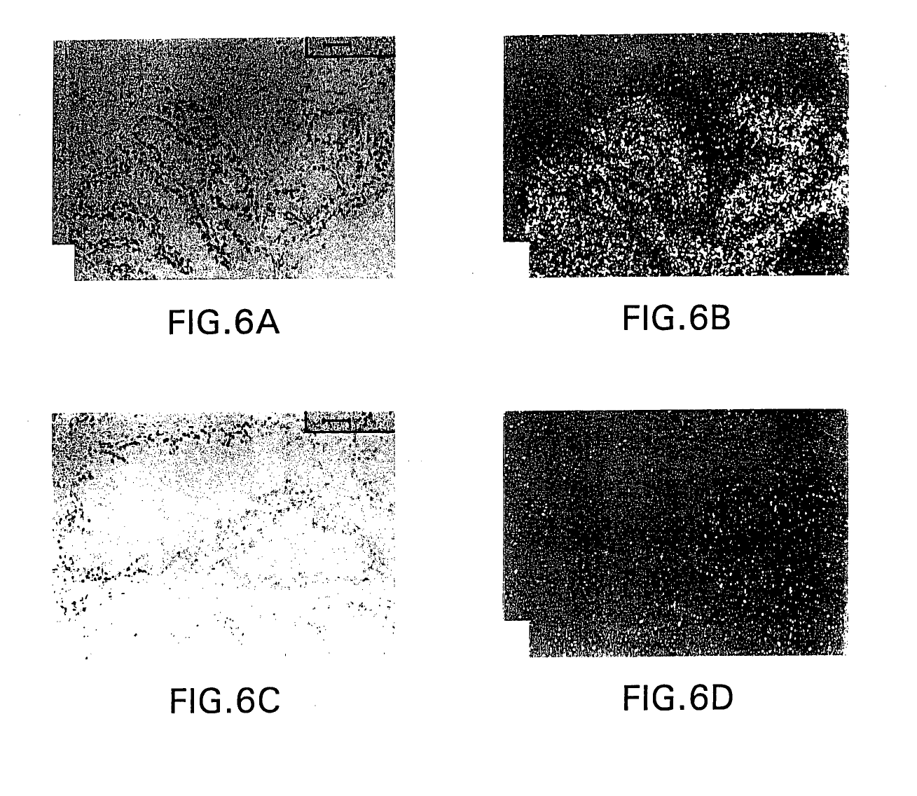

Furthermore, in situ hybridization was performed on frozen sections of human prostate to further test the hypothesis that PDEF expression is restricted to epithelial cells (See FIG. 6). In situ hybridization (ISH) was carried out by the following procedure: Tissues were fixed in 4% paraformaldehyde in phosphate buffered saline, pH 7.4 (PBS), for 2-4 h at 4° C. and were then transferred to 30% sucrose in PBS overnight at 4° C., frozen in OCT compound (Miles Diagnostics, Elkhart, Ind.) and stored at −70° C. ISH was performed on 6_m frozen sections. Slides were passed through xylene and graded alcohols; 0.2M HCl; Tris/EDTA with 3 mcg/ml proteinase K; 0.2% glycine; 4% paraformaldehyde in PBS; 0.1M triethanolamine containing 1/200 (vol/vol) acetic anhydride; and 2×SSC. Slides were hybridized overnight at 50° C. with 35S-labeled riboprobes in the following mixture: 0.3M NaCl, 0.01M Tris pH 7.6, 5 mM EDTA, 50% formamide, 10% dextran sulfate, 0.1 mg/ml yeast tRNA, and 0.01M dithiothreitol. Post-hybridization washes included 2×SSC/50% formamide/10 mM dithiothreitol at 50° C.; 4×SSC/10 mM Tris/1 mM EDTA with 20 mcg/ml ribonuclease at 37° C.; and 2×SSC/50% formamide/10 mM dithiothreitol at 65° C. and 2×SSC. Slides were then dehydrated through graded alcohols containing 0.3M ammonium acetate, dried, coated with Kodak NTB 2 emulsion and stored in the dark at 4° C. for 2 weeks. The emulsion was developed with Kodak D19 developer and the slides were counterstained with hematoxylin (French, C. C., Van de Water, L., Dvorak, H. F. and Hynes, R. O. J. Cell. Biol. 109: 903-914 (1989)).

Diffuse strong expression was only noted in luminal epithelium of the prostate, but not in other cell types. No signal was detected with sense control probe. These results further support the data that PDEF is exclusively expressed in epithelial cells, with particularly strong expression in the prostate.

The expression of PDEF from different cell types was determined by RT/PCR with mRNA derived from different cell types using both primary cells and cancer derived cell lines. RT-PCR was carried out by the following protocol: cDNAs were generated from 1 mg mRNA isolated from different cells or tissues using oligo dT12-18 priming (Gibco BRL Grand Island, N.Y. USA) and M-MLV reverse transcriptase (Gibco BRL) in deoxyribonuclease I (Gibco BRL) treated samples. Each PCR used equivalent amounts of (0.1 ng) cDNA, 4 ng/ul of each primer, 0.25 units of Taq polymerase (Promega, Madison, Wis. USA), 150 uM of each dNTP, 3 mM of MgCl2, reaction buffer and water to a final volume of 25 ul and were covered with mineral oil. The sequences of the primers for GAPDH were: sense: 5′-CAAAGTTGTCATGGATGACC-3′ (See SEQ ID NO.14) antisense: 5′CCATGGAGAAGGCTGGGG-3′ (See SEQ ID NO.15) with an expected amplification product of 200 bp. RT/PCR amplifications were carried out using a Perkin-Elmer Cetus thermal cycler 480 as follows: 20-30 cycles of 1 min at 94° C., 1 min at 56° C. and 1 min at 72° C. followed by 15 min at 72° C. Lower numbers of cycles were used to verify linearity of the amplification signal. The amplification product was analyzed on a 2% agarose gel.

Only cells derived from a prostate epithelial cancer, LNCaP, expressed high levels of PDEF mRNA, whereas other epithelial cells and non-epithelial cells were completely devoid of PDEF mRNA. Surprisingly, human aortic endothelial, but not umbilical vein endothelial cells expressed also low levels of PDEF. Thus, PDEF reveals a distinct and unique expression pattern, being restricted to primarily prostate epithelial cells. LNCaP cells were grown in T-medium (Gibco BRL, Formula No. 97-0295 DJ), 10% FCS, PEN/STREP, and were provided by Dr. Z. J. Sun, Dept. of Surgery and Genetics, Standford University School of Medicine.

### Example 4

### Chromosomal Mapping of PDEF

An oligonucleotide primer set is designed according to the sequence at the 5′ end of SEQ ID NO:1. This primer preferably spans about 100 nucleotides. This primer set is then used in a polymerase chain reaction under the following set of conditions : 30 seconds, 95° C.; 1 minute, 56° C.; 1 minute, 70° C. This cycle is repeated 32 times followed by one 5 minute cycle at 70° C. Human, mouse, and hamster DNA is used as template in addition to a somatic cell hybrid panel containing individual chromosomes or chromosome fragments (Bios, Inc). The reactions is analyzed on either 8% polyacrylamide gels or 3.5% agarose gels. Chromosome mapping is determined by the presence of an approximately 100 bp PCR fragment in the particular somatic cell hybrid.

PDEF localized in a region of human chromosome 6 that has been associated with loss of heterozygosity and chromosomal translocations in various human cancers. The precise chromosomal location of the PDEF gene was determined by single-gene fluorescence in situ hybridization to human chromosome metaphase spreads. Digitized images were analyzed, most of which had a doublet signal characteristic of genuine hybridization on both homologs of chromosome 6. Detailed analysis of individual chromosomes indicated that the PDEF gene is positioned within band 6p21, the majority of signals clustering in band 6p21.3.

Ets factors have been directly linked to tumorigenesis. Since the different members of this family either activate or repress gene expression, it is likely that some members of the Ets family act as oncogenes, whereas others as tumor suppressors. Aberrant expression of PDEF in cancer cells whose normal counterpart does not express PDEF has been observed. Chromosome 6p21.3 is of particular interest for various types of cancer. Non-random structural abnormalities of chromosome 6p21 are frequently observed in human tumors and have been linked to many human cancers including non-Hodgkin's lymphoma, follicle center lymphoma, ovarian cancer, multiple myeloma, cervical cancer, acute T-lymphoblastic leukemia, endometrial cancer, germ cell tumor, glioma, acute leukemias, CML, and melanoma. Since other Ets factors have been shown to be translocated in various types of tumors, it is possible that PDEF might be also involved in chromosomal abnormalities in human cancer.

### Example 5

### Bacterial Expression of PDEF

PDEF polynucleotide encoding a PDEF polypeptide invention is amplified using PCR oligonucleotide primers corresponding to the 5′ and 3′ ends of the DNA sequence, as outlined in Example 1, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Amp^{r}), a bacterial origin of replication (ori), an IPTG-regulatable promoter/operator (P/O), a ribosome binding site (RBS), a 6-histidine tag (6-His), and restriction enzyme cloning sites.

Specifically, to clone the PDEF protein in a bacterial vector, both a 5′ and 3′ primer of a sequence specific to the terminal regions of the PDEF sequence in SEQ ID NO:1 is designed. One of ordinary skill in the art would appreciate, of course, that the point in the protein coding sequence where the 5′ primer begins may be varied to amplify a DNA segment encoding any desired portion of the complete PDEF protein shorter or longer than the full-length protein. Moreover, described primers could then be used to amplify the corresponding cDNA PDEF clone (SEQ ID NO:1) by PCR methodology—taking advantage of restriction sites present within the bacterial vector and added to the terminal ends of the corresponding PDEF-specific primers.

For example, the pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, which expresses the lacI repressor and also confers kanamycin resistance (Kan^{r}). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.⁶⁰⁰) of between 0.4 and 0.6. IPTG (Isopropyl-B-D-thiogalacto pyranoside) is then added to a final concentration of 1 mM. IPTG induces by inactivating the lacd repressor, clearing the P/O leading to increased gene expression.

Cells are grown for an extra 3 to 4 hours. Cells are then harvested by centrifugation (20 mins at 6000×g). The cell pellet is solubilized in the chaotropic agent 6 Molar Guanidine HCl by stirring for 3-4 hours at 4° C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6×His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified PDEF protein is then renatured by dialyzing it against phosphate-buffered saline (PBS) or 50 mM Na-acetate, pH 6 buffer plus 200 mM NaCl. Alternatively, the PDEF protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM immidazole. Immidazole is removed by a final dialyzing step against PBS or 50 mM sodium acetate pH 6 buffer plus 200 mM NaCl. The purified PDEF protein is stored at 40° C. or frozen at −80° C.

In addition to the above expression vector, the present invention further includes an expression vector comprising phage operator and promoter elements operatively linked to a PDEF polynucleotide, called pHE4a. (ATCC Accession Number 209645, deposited Feb. 25, 1998.) This vector contains: 1) a neomycinphosphotransferase gene as a selection marker, 2) an E. coli origin of replication, 3) a T5 phage promoter sequence, 4) two lac operator sequences, 5) a Shine-Delgarno sequence, and 6) the lactose operon repressor gene (lacIq). The origin of replication (oriC) is derived from pUC19 (LTI, Gaithersburg, Md.). The promoter sequence and operator sequences are made synthetically.

DNA can be inserted into the pHEa by restricting the vector with NdeI and XbaI, BamHI, XhoI, or Asp718, running the restricted product on a gel, and isolating the larger fragment (the stuffer fragment should be about 310 base pairs). The DNA insert is generated according to the PCR protocol described in Example 1, using PCR primers having restriction sites for NdeI (5′ primer) and XbaI, BamHI, XhoI, or Asp718 (3′ primer). The PCR insert is gel purified and restricted with compatible enzymes. The insert and vector are ligated according to standard protocols.

The engineered vector could easily be substituted in the above protocol to express protein in a bacterial system.

### Example 6

### Purification of PDEF Polypeptide from an Inclusion Body

The following alternative method can be used to purify PDEF polypeptide expressed in E coli when it is present in the form of inclusion bodies. Unless otherwise specified, all of the following steps are conducted at 4-10° C.

Upon completion of the production phase of the E. coli fermentation, the cell culture is cooled to 4-10° C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifligation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the polypeptide containing supernatant is incubated at 4° C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded polypeptide solution, a previously prepared tangential filtration unit equipped with 0.16 μm membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the PDEF polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the polypeptide (determined, for instance, by 16% SDS-PAGE) are then pooled.

The resultant PDEF polypeptide should exhibit greater than 95% purity after the above refolding and purification steps. No major contaminant bands should be observed from Commassie blue stained 16% SDS-PAGE gel when 5 μg of purified protein is loaded. The purified PDEF protein can also be tested for endotoxin/LPS contamination, and typically the LPS content is less than 0.1 ng/ml according to LAL assays.

### Example 7

### Cloning and Expression of PDEF in a Baculovirus Expression System

In this example, the plasmid shuttle vector pA2 is used to insert PDEF polynucleotide into a baculovirus to express PDEF. This expression vector contains the strong polyhedrin promoter of the Autographa californica nuclear polyhedrosis virus (AcMNPV) followed by convenient restriction sites such as BamHI, Xba I and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned PDEF polynucleotide.

Many other baculovirus vectors can be used in place of the vector above, such as pAc373, pVL941, and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

Specifically, the PDEF cDNA sequence contained in the deposited clone, including the AUG initiation codon is amplified using the PCR protocol described in Example 1. Alternatively, the vector can be modified (pA2 GP) to include a baculovirus leader sequence, using the standard methods described in Summers et al., “A Manual of Methods for Baculovirus Vectors and Insect Cell Culture Procedures,” Texas Agricultural Experimental Station Bulletin No. 1555 (1987).

More specifically, the cDNA sequence encoding the full length PDEF protein in the deposited clone, including the AUG initiation codon, shown in SEQ ID NO:1, is amplified using PCR oligonucleotide primers corresponding to the 5′ and 3′ sequences of the gene.

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five μg of a plasmid containing the polynucleotide is co-transfected with 1.0 μg of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84:7413-7417 (1987). One μg of BaculoGold™ virus DNA and 5 μg of the plasmid are mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supematants of these culture dishes are harvested and then they are stored at 4° C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 μCi of ³⁵S-methionine and 5 μCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced PDEF protein.

### Example 8

### Expression of PDEF in Mammalian Cells

PDEF polypeptide can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, PDEF polypeptide can be expressed in stable cell lines containing the PDEF polynucleotide integrated into a chromosome. The co-transfection with a selectable marker such as DHFR, GPT, neomycin, hygromycin allows the identification and isolation of the transfected cells.

The transfected PDEF gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

Derivatives of the plasmid pSV2-DHFR (ATCC Accession No. 37146), the expression vectors pC4 (ATCC Accession No. 209646) and pC6 (ATCC Accession No.209647) contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985).) Multiple cloning sites, e.g., with the restriction enzyme cleavage sites BamHI, XbaI and Asp718, facilitate the cloning of PDEF. The vectors also contain the 3′ intron, the polyadenylation and termination signal of the rat preproinsulin gene, and the mouse DHFR gene under control of the SV40 early promoter.

Specifically, the plasmid pC6, for example, is digested with appropriate restriction enzymes and then dephosphorylated using calf intestinal phosphates by procedures known in the art. The vector is then isolated from a 1% agarose gel.

PDEF polynucleotide is amplified according to the protocol outlined in Example 1. Because PDEF is not naturally secreted, the vector does not need a signal peptide. Alternatively, if the naturally occurring signal sequence is not used, the vector can be modified to include a heterologous signal sequence. (See, e.g., WO 96/34891.)

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transfection. Five μg of the expression plasmid pC6 is cotransfected with 0.5 μg of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of PDEF is analyzed, for instance, by SDS-PAGE and Western blot or by reversed phase HPLC analysis.

### Example 9

### Construction of N-Terminal and/or C-Terminal Deletion Mutants

The following general approach may be used to clone a N-terminal or C-terminal deletion PDEF deletion mutant. Generally, two oligonucleotide primers of about 15-25 nucleotides are derived from the desired 5′ and 3′ positions of a polynucleotide of SEQ ID NO:1. The 5′ and 3′ positions of the primers are determined based on the desired PDEF polynucleotide fragment. An initiation and stop codon are added to the 5′ and 3′ primers respectively, if necessary, to express the PDEF polypeptide fragment encoded by the polynucleotide fragment. Preferred PDEF polynucleotide fragments are those encoding the N-terminal and C-terminal deletion mutants disclosed above in the “Polynucleotide and Polypeptide Fragments” section of the Specification.

Additional nucleotides containing restriction sites to facilitate cloning of the PDEF polynucleotide fragment in a desired vector may also be added to the 5′ and 3′ primer sequences. The PDEF polynucleotide fragment is amplified from genomic DNA or from the deposited cDNA clone using the appropriate PCR oligonucleotide primers and conditions discussed herein or known in the art. The PDEF polypeptide fragments encoded by the PDEF polynucleotide fragments of the present invention may be expressed and purified in the same general manner as the full length polypeptides, although routine modifications may be necessary due to the differences in chemical and physical properties between a particular fragment and full length polypeptide.

As a means of exemplifying but not limiting the present invention, the polynucleotide encoding the PDEF polypeptide fragment P-248 to V-332 is amplified and cloned as follows: A 5′ primer is generated comprising a restriction enzyme site followed by an initiation codon in frame with the polynucleotide sequence encoding the N-terminal portion of the polypeptide fragment beginning with a start codon of P representing amino acid 248 in SEQ ID NO:2. A complementary 3′ primer is generated comprising a restriction enzyme site followed by a stop codon in frame with the polynucleotide sequence encoding C-terminal portion of the PDEF polypeptide fragment ending with a V which represents the amino acid 332 in SEQ ID NO:2.

The amplified polynucleotide fragment and the expression vector are digested with restriction enzymes which recognize the sites in the primers. The digested polynucleotides are then ligated together. The PDEF polynucleotide fragment is inserted into the restricted expression vector, preferably in a manner which places the PDEF polypeptide fragment coding region downstream from the promoter. The ligation mixture is transformed into competent E. coli cells using standard procedures and as described in the Examples herein. Plasmid DNA is isolated from resistant colonies and the identity of the cloned DNA confirmed by restriction analysis, PCR and DNA sequencing.

### Example 10

### Protein Fusions of PDEF

PDEF polypeptides are preferably fused to other proteins. These fusion proteins can be used for a variety of applications. For example, fusion of PDEF polypeptides to His-tag, HA-tag, protein A, IgG domains, and maltose binding protein facilitates purification. (See Example 5; see also EP A 394,827; Traunecker, et al., Nature 331:84-86 (1988).) Similarly, fusion to IgG-1, IgG-3, and albumin increases the halflife time in vivo. Nuclear localization signals fused to PDEF polypeptides can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule, or the protocol described in Example 5.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (Accession No. 209646) is used, the human Fc portion can be ligated into the BamHI cloning site. Note that the 3′ BamHI site should be destroyed. Next, the vector containing the human Fc portion is re-restricted with BamHI, linearizing the vector, and PDEF polynucleotide, isolated by the PCR protocol described in Example 1, is ligated into this BamHI site. Note that the polynucleotide is cloned without a stop codon, otherwise a fusion protein will not be produced.

The vector can be modified to include a heterologous signal sequence if a secreted protein is to be made. (See, e.g., WO 96/34891.)

Human IgG Fc region:

| | |
|---|---|
| GGGATCCGGAGCCCAAATCTTCTGACAAAACTCACACATGCCCACCGTGCCCAGC | (SEQ ID NO:4) |
| ACCTGAATTCGAGGGTGCACCGTCAGTCTTCCTCTTCCCCCCAAAACCCAAGGAC | |
| ACCCTCATGATCTCCCGGACTCCTGAGGTCACATGCGTGGTGGTGGACGTAAGCC | |
| ACGAAGACCCTGAGGTCAAGTTCAACTGGTACGTGGACGGCGTGGAGGTGCATA | |
| ATGCCAAGACAAAGCCGCGGGAGGAGCAGTACAACAGCACGTACCGTGTGGTCA | |
| GCGTCCTCACCGTCCTGCACCAGGACTGGCTGAATGGCAAGGAGTACAAGTGCA | |
| AGGTCTCCAACAAAGCCCTCCCAACCCCCATCGAGAAAACCATCTCCAAAGCCA | |
| AAGGGCAGCCCCGAGAACCACAGGTGTACACCCTGCCCCCATCCCGGGATGAGC | |
| TGACCAAGAACCAGGTCAGCCTGACCTGCCTGGTCAAAGGCTTCTATCCAAGCGA | |
| CATCGCCGTGGAGTGGGAGAGCAATGGGCAGCCGGAGAACAACTACAAGACCAC | |
| GCCTCCCGTGCTGGACTCCGACGGCTCCTTCTTCCTCTACAGCAAGCTCACCGTGG | |
| ACAAGAGCAGGTGGCAGCAGGGGAACGTCTTCTCATGCTCCGTGATGCATGAGG | |
| CTCTGCACAACCACTACACGCAGAAGAGCCTCTCCCTGTCTCCGGGTAAATGAGT | |
| GCGACGGCCGCGACTCTAGAGGAT | |

### Example 11

### Production of an Antibody

The antibodies of the present invention can be prepared by a variety of methods. (See, Current Protocols, Chapter 2.) For example, cells expressing PDEF is administered to an animal to induce the production of sera containing polyclonal antibodies. In a preferred method, a preparation of PDEF protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Köhler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with PDEF polypeptide or, more preferably, with a secreted PDEF polypeptide-expressing cell. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP2O), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the PDEF polypeptide.

Alternatively, additional antibodies capable of binding to PDEF polypeptide can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the PDEF protein-specific antibody can be blocked byPDEF. Such antibodies comprise anti-idiotypic antibodies to the PDEF protein-specific antibody and can be used to immunize an animal to induce formation of further PDEF protein-specific antibodies.

It will be appreciated that Fab and F(ab′)2 and other fragments of the antibodies of the present invention maybe used according to the methods disclosed herein. Such fragments are typically produced by proteolytic cleavage, using enzymes such as papain (to produce Fab fragments) or pepsin (to produce F(ab′)2 fragments). Alternatively, secreted PDEF protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

### Example 12

### Production of PDEF Protein for High-Throughput Screening Assays

The following protocol produces a supernatant containing PDEF polypeptide to be tested, or proteins produced by the binding of PDEF to the gene promoters. This supernatant can then be used in the Screening Assays described in Examples 14-21.

First, dilute Poly-D-Lysine (644 587 Boehringer-Mannheim) stock solution (1 mg/ml in PBS) 1:20 in PBS (w/o calcium or magnesium 17-516F Biowhittaker) for a working solution of 50 μg/ml. Add 200 μl of this solution to each well (24 well plates) and incubate at RT for 20 minutes. Be sure to distribute the solution over each well (note: a 12-channel pipetter may be used with tips on every other channel). Aspirate off the Poly-D-Lysine solution and rinse with 1 ml PBS (Phosphate Buffered Saline). The PBS should remain in the well until just prior to plating the cells and plates may be poly-lysine coated in advance for up to two weeks.

Plate 293T cells (do not carry cells past P+20) at 2×10⁵ cells/well in 0.5 ml DMEM(Dulbecco's Modified Eagle Medium)(with 4.5 G/L glucose and L-glutamine (12-604F Biowhittaker))/10% heat inactivated FBS(14-503F Biowhittaker)/1× Penstrep(17-602E Biowhittaker). Let the cells grow overnight.

The next day, mix together in a sterile solution basin: 300 ul Lipofectamine (18324-012 Gibco/BRL) and 5 ml Optimem I(31985070 Gibco/BRL)/96-well plate. With a small volume multi-channel pipetter, aliquot approximately 2 μg of an expression vector containing a polynucleotide insert, produced by the methods described in Examples 8-10, into an appropriately labeled 96-well round bottom plate. With a multi-channel pipetter, add 50 μl of the Lipofectamine/Optimem I mixture to each well. Pipette up and down gently to mix. Incubate at RT 15-45 minutes. After about 20 minutes, use a multi-channel pipetter to add 150 μl Optimem I to each well. As a control, one plate of vector DNA lacking an insert should be transfected with each set of transfections.

Preferably, the transfection should be performed by tag-teaming the following tasks. By tag-teaming, hands on time is cut in half, and the cells do not spend too much time on PBS. First, person A aspirates off the media from four 24-well plates of cells, and then person B rinses each well with 0.5-1 ml PBS. Person A then aspirates off PBS rinse, and person B, using a 12-channel pipetter with tips on every other channel, adds the 200 μl of DNA/Lipofectamine/Optimem I complex to the odd wells first, then to the even wells, to each row on the 24-well plates. Incubate at 37° C. for 6 hours.

While cells are incubating, prepare appropriate media, either 1% BSA in DMEM with 1× penstrep, or HGS CHO-5 media (116.6 mg/L of CaCl2 (anhyd); 0.00130 mg/L CuSO₄-5H₂O; 0.050 mg/L of Fe(NO₃)₃-9H₂O; 0.417 mg/L of FeSO₄-7H₂O; 311.80 mg/L of Kcl; 28.64 mg/L of MgCl₂; 48.84 mg/L of MgSO₄; 6995.50 mg/L of NaCl; 2400.0 mg/L of NaHCO₃; 62.50 mg/L of NaH₂PO₄-H₂O; 71.02 mg/L of Na₂HPO4; 0.4320 mg/L of ZnSO₄-7H₂O; 0.002 mg/L of Arachidonic Acid; 1.022 mg/L of Cholesterol; 0.070 mg/L of DL-alpha-Tocopherol-Acetate; 0.0520 mg/L of Linoleic Acid; 0.010 mg/L of Linolenic Acid; 0.010 mg/L of Myristic Acid; 0.010 mg/L of Oleic Acid; 0.010 mg/L of Palmitric Acid; 0.010 mg/L of Palmitic Acid; 100 mg/L of Pluronic F-68; 0.010 mg/L of Stearic Acid; 2.20 mg/L of Tween 80; 4551 mg/L of D-Glucose; 130.85 mg/ml of L-Alanine; 147.50 mg/ml of L-Arginine-HCL; 7.50 mg/ml of L-Asparagine-H₂O; 6.65 mg/ml of L-Aspartic Acid; 29.56 mg/ml of L-Cystine-2HCL-H₂O; 31.29 mg/ml of L-Cystine-2HCL; 7.35 mg/ml of L-Glutamic Acid; 365.0 mg/ml of L-Glutamine; 18.75 mg/ml of Glycine; 52.48 mg/ml of L-Histidine-HCL-H₂O; 106.97 mg/ml of L-Isoleucine; 111.45 mg/ml of L-Leucine; 163.75 mg/ml of L-Lysine HCL; 32.34 mg/ml of L-Methionine; 68.48 mg/ml of L-Phenylalainine; 40.0 mg/ml of L-Proline; 26.25 mg/ml of L-Serine; 101.05 mg/ml of L-Threonine; 19.22 mg/ml of L-Tryptophan; 91.79 mg/ml of L-Tryrosine-2Na-2H₂O; and 99.65 mg/ml of L-Valine; 0.0035 mg/L of Biotin; 3.24 mg/L of D-Ca Pantothenate; 11.78 mg/L of Choline Chloride; 4.65 mg/L of Folic Acid; 15.60 mg/L of i-Inositol; 3.02 mg/L of Niacinamide; 3.00 mg/L of Pyridoxal HCL; 0.031 mg/L of Pyridoxine HCL; 0.319 mg/L of Riboflavin; 3.17 mg/L of Thiamine HCL; 0.365 mg/L of Thymidine; 0.680 mg/L of Vitamin B₁₂; 25 mM of HEPES Buffer; 2.39 mg/L of Na Hypoxanthine; 0.105 mg/L of Lipoic Acid; 0.081 mg/L of Sodium Putrescine-2HCL; 55.0 mg/L of Sodium Pyruvate; 0.0067 mg/L of Sodium Selenite; 20 uM of Ethanolamine; 0.122 mg/L of Ferric Citrate; 41.70 mg/L of Methyl-B-Cyclodextrin complexed with Linoleic Acid; 33.33 mg/L of Methyl-B-Cyclodextrin complexed with Oleic Acid; 10 mg/L of Methyl-B-Cyclodextrin complexed with Retinal Acetate. Adjust osmolarity to 327 mOsm) with 2 mm glutamine and 1× penstrep. (BSA (81-068-3 Bayer) 100 gm dissolved in 1 L DMEM for a 10% BSA stock solution). Filter the media and collect 50 ul for endotoxin assay in 15 ml polystyrene conical.

The transfection reaction is terminated, preferably by tag-teaming, at the end of the incubation period. Person A aspirates off the transfection media, while person B adds 1.5 ml appropriate media to each well. Incubate at 37° C. for 45 or 72 hours depending on the media used: 1% BSA for 45 hours or CHO-5 for 72 hours.

On day four, using a 300 ul multichannel pipetter, aliquot 600 ul in one 1 ml deep well plate and the remaining supernatant into a 2 ml deep well. The supernatants from each well can then be used in the assays described in Examples 14-21.

It is specifically understood that when activity is obtained in any of the assays described below using a supernatant, the activity originates from either the PDEF polypeptide directly (e.g., as a secreted and/or soluble protein) or by PDEF inducing expression of other proteins, which are then secreted into the supernatant. Thus, the invention further provides a method of identifying the protein in the supernatant characterized by an activity in a particular assay.

### Example 13

### Construction of GAS Reporter Construct

One signal transduction pathway involved in the differentiation and proliferation of cells is called the Jaks-STATs pathway. Activated proteins in the Jaks-STATs pathway bind to gamma activation site “GAS” elements or interferon-sensitive responsive element (“ISRE”), located in the promoter of many genes. The binding of a protein to these elements alter the expression of the associated gene.

GAS and ISRE elements are recognized by a class of transcription factors called Signal Transducers and Activators of Transcription, or “STATs.” There are six members of the STATs family. Stat1 and Stat3 are present in many cell types, as is Stat2 (as response to IFN-alpha is widespread). Stat4 is more restricted and is not in many cell types though it has been found in T helper class I, cells after treatment with IL-12. Stat5 was originally called mammary growth factor, but has been found at higher concentrations in other cells including myeloid cells. It can be activated in tissue culture cells by many cytokines.

The STATs are activated to translocate from the cytoplasm to the nucleus upon tyrosine phosphorylation by a set of kinases known as the Janus Kinase (“Jaks”) family. Jaks represent a distinct family of soluble tyrosine kinases and include Tyk2, Jak1, Jak2, and Jak3. These kinases display significant sequence similarity and are generally catalytically inactive in resting cells.

The Jaks are activated by a wide range of receptors summarized in the Table below. (Adapted from review by Schidler and Damell, Ann. Rev. Biochem. 64:621-51 (1995).) A cytokine receptor family, capable of activating Jaks, is divided into two groups: (a) Class 1 includes receptors for IL-2, IL-3, IL-4, IL-6, IL-7, IL-9, IL-11, IL-12, IL-15, Epo, PRL, GH, G-CSF, GM-CSF, LIF, CNTF, and thrombopoietin; and (b) Class 2 includes IFN-a, IFN-g, and IL-10. The Class 1 receptors share a conserved cysteine motif (a set of four conserved cysteines and one tryptophan) and a WSXWS motif (a membrane proxial region encoding Trp-Ser-Xxx-Trp-Ser (SEQ ID NO:5)).

Thus, on binding of a ligand to a receptor, Jaks are activated, which in turn activate STATs, which then translocate and bind to GAS elements. This entire process is encompassed in the Jaks-STATs signal transduction pathway.

Therefore, activation of the Jaks-STATs pathway, reflected by the binding of the GAS or the ISRE element, can be used to indicate proteins involved in the proliferation and differentiation of cells. For example, growth factors and cytokines are known to activate the Jaks-STATs pathway. (See Table below.) Thus, by using GAS elements linked to reporter molecules, activators of the Jaks-STATs pathway can be identified.

| | | JAKs | | | | |
|---|---|---|---|---|---|---|
| Ligand | tyk2 | Jak1 | Jak2 | Jak3 | STATS | GAS (elements) or ISRE |
| IFN family | | | | | | |
| IFN-a/B | + | + | − | − | 1, 2, 3 | ISRE |
| IFN-g | | + | + | − | 1 | GAS (IRF1 > Lys6 > IFP) |
| Il-10 | + | ? | ? | − | 1, 3 | |
| gp130 family | | | | | | |
| IL-6 (Pleiotropic) | + | + | + | ? | 1, 3 | GAS (IRF1 > Lys6 > IFP) |
| Il-11 (Pleiotropic) | ? | + | ? | ? | 1, 3 | |
| OnM (Pleiotropic) | ? | + | + | ? | 1, 3 | |
| LIF (Pleiotropic) | ? | + | + | ? | 1, 3 | |
| CNTF (Pleiotropic) | −/+ | + | + | ? | 1, 3 | |
| G-CSF (Pleiotropic) | ? | + | ? | ? | 1, 3 | |
| IL-12 (Pleiotropic) | + | − | + | + | 1, 3 | |
| g-C family | | | | | | |
| IL-2 (lymphocytes) | − | + | − | + | 1, 3, 5 | GAS |
| IL-4 (lymph/myeloid) | − | + | − | + | 6 | GAS (IRF1 = IFP >> Ly6)(IgH) |
| IL-7 (lymphocytes) | − | + | − | + | 5 | GAS |
| IL-9 (lymphocytes) | − | + | − | + | 5 | GAS |
| IL-13 (lymphocyte) | − | + | ? | ? | 6 | GAS |
| IL-15 | ? | + | ? | + | 5 | GAS |
| gp140 family | | | | | | |
| IL-3 (myeloid) | − | − | + | − | 5 | GAS (IRF1 > IFP >> Ly6) |
| IL-5 (myeloid) | − | − | + | − | 5 | GAS |
| GM-CSF (myeloid) | − | − | + | − | 5 | GAS |
| Growth hormone family | | | | | | |
| GH | ? | − | + | − | 5 | |
| PRL | ? | +/− | + | − | 1, 3, 5 | |
| EPO | ? | − | + | − | 5 | GAS (B-CAS > IRF1 = IFP >> Ly6) |
| Receptor Tyrosine Kinases | | | | | | |
| EGF | ? | + | + | − | 1, 3 | GAS (IRF1) |
| PDGF | ? | + | + | − | 1, 3 | |
| CSF-1 | ? | + | + | − | 1, 3 | GAS (not IRF1) |

To construct a synthetic GAS containing promoter element, which is used in the Biological Assays described in Examples 14-15, a PCR based strategy is employed to generate a GAS-SV40 promoter sequence. The 5′ primer contains four tandem copies of the GAS binding site found in the IRF1 promoter and previously demonstrated to bind STATs upon induction with a range of cytokines (Rothman et al., Immunity 1:457-468 (1994).), although other GAS or ISRE elements can be used instead. The 5′ primer also contains 18 bp of sequence complementary to the SV40 early promoter sequence and is flanked with an XhoI site. The sequence of the 5′ primer is:

| | |
|---|---|
| 5′:GCGCCTCGAGATTTCCCCGAAATCTAGATTTCCCCGAAATGATTTCCCCGAAAT | (SEQ ID NO:6) |
| GATTTCCCCGAAATATCTGCCATCTCAATTAG:3′ | |

The downstream primer is complementary to the SV40 promoter and is flanked with a Hind III site: 5′:GCGGCAAGCTTTTTGCAAAGCCTAGGC:3′ (SEQ ID NO:7)

PCR amplification is performed using the SV40 promoter template present in the B-gal:promoter plasmid obtained from Clontech. The resulting PCR fragment is digested with XhoI/Hind III and subcloned into BLSK2-. (Stratagene.) Sequencing with forward and reverse primers confirms that the insert contains the following sequence:

| | |
|---|---|
| 5′:CTCGAGATTTCCCCGAAATCTAGATTTCCCCGAAATGATTTCCCCGAAATGATT | (SEQ ID NO:8) |
| TCCCCGAAATATCTGCCATCTCAATTAGTCAGCAACCATAGTCCCGCCCCTAACT | |
| CCGCCCATCCCGCCCCTAACTCCGCCCAGTTCCGCCCATTCTCCGCCCCATGGCTG | |
| ACTAATTTTTTTTATTTATGCAGAGGCCGAGGCCGCCTCGGCCTCTGAGCTATTCC | |
| AGAAGTAGTGAGGAGGCTTTTTTGGAGGCCTAGGCTTTTGCAAAAAGCTT:3′ | |

With this GAS promoter element linked to the SV40 promoter, a GAS:SEAP2 reporter construct is next engineered. Here, the reporter molecule is a secreted alkaline phosphatase, or “SEAP.” Clearly, however, any reporter molecule can be instead of SEAP, in this or in any of the other Examples. Well known reporter molecules that can be used instead of SEAP include chloramphenicol acetyltransferase (CAT), luciferase, alkaline phosphatase, B-galactosidase, green fluorescent protein (GFP), or any protein detectable by an antibody.

The above sequence confirmed synthetic GAS-SV40 promoter element is subcloned into the pSEAP-Promoter vector obtained from Clontech using HindIII and XhoI, effectively replacing the SV40 promoter with the amplified GAS:SV40 promoter element, to create the GAS-SEAP vector. However, this vector does not contain a neomycin resistance gene, and therefore, is not preferred for mammalian expression systems.

Thus, in order to generate mammalian stable cell lines expressing the GAS-SEAP reporter, the GAS-SEAP cassette is removed from the GAS-SEAP vector using SalI and NotI, and inserted into a backbone vector containing the neomycin resistance gene, such as pGFP-1 (Clontech), using these restriction sites in the multiple cloning site, to create the GAS-SEAP/Neo vector. Once this vector is transfected into mammalian cells, this vector can then be used as a reporter molecule for GAS binding as described in Examples 14-15.

Other constructs can be made using the above description and replacing GAS with a different promoter sequence. For example, construction of reporter molecules containing NFK-B and EGR promoter sequences are described in Examples 16 and 17. However, many other promoters can be substituted using the protocols described in these Examples. For instance, SRE, IL-2, NFAT, or Osteocalcin promoters can be substituted, alone or in combination (e.g., GAS/NF-KB/EGR, GAS/NF-KB, I1-2/NFAT, or NF-KB/GAS). Similarly, other cell lines can be used to test reporter construct activity, such as HELA (epithelial), HUVEC (endothelial), Reh (B-cell), Saos-2 (osteoblast), HUVAC (aortic), or Cardiomyocyte.

### Example 14

### High-Throughput Screening Assay for T-cell Activity

The following protocol is used to assess T-cell activity of PDEF by determining whether PDEF supernatant proliferates and/or differentiates T-cells. T-cell activity is assessed using the GAS/SEAP/Neo construct produced in Example 13. Thus, factors that increase SEAP activity indicate the ability to activate the Jaks-STATS signal transduction pathway. The T-cell used in this assay is Jurkat T-cells (ATCC Accession No. TIB-152), although Molt-3 cells (ATCC Accession No. CRL-1552) and Molt-4 cells (ATCC Accession No. CRL-1582) cells can also be used.

Jurkat T-cells are lymphoblastic CD4+ Th1 helper cells. In order to generate stable cell lines, approximately 2 million Jurkat cells are transfected with the GAS-SEAP/neo vector using DMRIE-C (Life Technologies)(transfection procedure described below). The transfected cells are seeded to a density of approximately 20,000 cells per well and transfectants resistant to 1 mg/ml genticin selected. Resistant colonies are expanded and then tested for their response to increasing concentrations of interferon gamma. The dose response of a selected clone is demonstrated.

Specifically, the following protocol will yield sufficient cells for 75 wells containing 200 ul of cells. Thus, it is either scaled up, or performed in multiple to generate sufficient cells for multiple 96 well plates. Jurkat cells are maintained in RPMI+10% serum with 1% Pen-Strep. Combine 2.5 mls of OPTI-MEM (Life Technologies) with 10 ug of plasmid DNA in a T25 flask. Add 2.5 ml OPTI-MEM containing 50 ul of DMRIE-C and incubate at room temperature for 15-45 mins.

During the incubation period, count cell concentration, spin down the required number of cells (10⁷ per transfection), and resuspend in OPTI-MEM to a final concentration of 10⁷ cells/ml. Then add 1 ml of 1×10⁷ cells in OPTI-MEM to T25 flask and incubate at 37° C. for 6 hrs. After the incubation, add 10 ml of RPMI+15% serum.

The Jurkat:GAS-SEAP stable reporter lines are maintained in RPMI+10% serum, 1 mg/ml Genticin, and 1% Pen-Strep. These cells are treated with supernatants containing PDEF polypeptides or PDEF induced polypeptides as produced by the protocol described in Example 12.

On the day of treatment with the supernatant, the cells should be washed and resuspended in fresh RPMI+10% serum to a density of 500,000 cells per ml. The exact number of cells required will depend on the number of supernatants being screened. For one 96 well plate, approximately 10 million cells (for 10 plates, 100 million cells) are required.

Transfer the cells to a triangular reservoir boat, in order to dispense the cells into a 96 well dish, using a 12 channel pipette. Using a 12 channel pipette, transfer 200 ul of cells into each well (therefore adding 100,000 cells per well).

After all the plates have been seeded, 50 ul of the supernatants are transferred directly from the 96 well plate containing the supernatants into each well using a 12 channel pipette. In addition, a dose of exogenous interferon gamma (0.1, 1.0, 10 ng) is added to wells H9, H10, and H11 to serve as additional positive controls for the assay.

The 96 well dishes containing Jurkat cells treated with supernatants are placed in an incubator for 48 hrs (note: this time is variable between 48-72 hrs). 35 ul samples from each well are then transferred to an opaque 96 well plate using a 12 channel pipette. The opaque plates should be covered (using sellophene covers) and stored at −20° C. until SEAP assays are performed according to Example 18. The plates containing the remaining treated cells are placed at 4° C. and serve as a source of material for repeating the assay on a specific well if desired.

As a positive control, 100 Unit/ml interferon gamma can be used which is known to activate Jurkat T cells. Over 30 fold induction is typically observed in the positive control wells.

### Example 15

### High-Throughput Screening Assay Identifying Myeloid Activity

The following protocol is used to assess myeloid activity of PDEF by determining whether PDEF proliferates and/or differentiates myeloid cells. Myeloid cell activity is assessed using the GAS/SEAP/Neo construct produced in Example 13. Thus, factors that increase SEAP activity indicate the ability to activate the Jaks-STATS signal transduction pathway. The myeloid cell used in this assay is U937, a pre-monocyte cell line, although TF-1, HL60, or KG1 can be used.

To transiently transfect U937 cells with the GAS/SEAP/Neo construct produced in Example 13, a DEAE-Dextran method (Kharbanda et. al., 1994, Cell Growth & Differentiation, 5:259-265) is used. First, harvest 2×10e⁷ U937 cells and wash with PBS. The U937 cells are usually grown in RPMI 1640 medium containing 10% heat-inactivated fetal bovine serum (FBS) supplemented with 100 units/ml penicillin and 100 mg/ml streptomycin.

Next, suspend the cells in 1 ml of 20 mM Tris-HCl (pH 7.4) buffer containing 0.5 mg/ml DEAE-Dextran, 8 ug GAS-SEAP2 plasmid DNA, 140 mM NaCl, 5 mM KCl, 375 μM Na₂HPO₄.7H₂O, 1 mM MgCl₂, and 675 μM CaCl₂. Incubate at 37° C. for 45 min.

Wash the cells with RPMI 1640 medium containing 10% FBS and then resuspend in 10 ml complete medium and incubate at 37° C. for 36 hr.

The GAS-SEAP/U937 stable cells are obtained by growing the cells in 400 ug/ml G418. The G418-free medium is used for routine growth but every one to two months, the cells should be re-grown in 400 ug/ml G418 for couple of passages.

These cells are tested by harvesting 1×10⁸ cells (this is enough for ten 96-well plates assay) and wash with PBS. Suspend the cells in 200 ml above described growth medium, with a final density of 5×10⁵ cells/ml. Plate 200 ul cells per well in the 96-well plate (or 1×10⁵ cells/well).

Add 50 ul of the supernatant prepared by the protocol described in Example 12. Incubate at 37° C. for 48 to 72 hr. As a positive control, 100 Unit/ml interferon gamma can be used which is known to activate U937 cells. Over 30 fold induction is typically observed in the positive control wells. SEAP assay the supernatant according to the protocol described in Example 18.

### Example 16

### High-Throughput Screening Assay Identifying Neuronal Activity.

When cells undergo differentiation and proliferation, a group of genes are activated through many different signal transduction pathways. One of these genes, EGR1 (early growth response gene 1), is induced in various tissues and cell types upon activation. The promoter of EGR1 is responsible for such induction. Using the EGR1 promoter linked to reporter molecules, activation of cells can be assessed by PDEF.

Particularly, the following protocol is used to assess neuronal activity in PCI2 cell lines. PCI2 cells (rat phenochromocytoma cells) are known to proliferate and/or differentiate by activation with a number of mitogens, such as TPA (tetradecanoyl phorbol acetate), NGF (nerve growth factor), and EGF (epidermal growth factor). The EGR1 gene expression is activated during this treatment. Thus, by stably transfecting PCI2 cells with a construct containing an EGR promoter linked to SEAP reporter, activation of PCI2 cells by PDEF can be assessed.

The EGR/SEAP reporter construct can be assembled by the following protocol. The EGR-1 promoter sequence (−633 to +1)(Sakamoto K et al., Oncogene 6:867-871 (1991)) can be PCR amplified from human genomic DNA using the following primers:

| | |
|---|---|
| 5′ GCGCTCGAGGGATGACAGCGATAGAACCCCGG-3′ | (SEQ ID |
| | NO:9) |
| 5′ GCGAAGCTTCGCGACTCCCCGGATCCGCCTC-3′ | (SEQ ID |
| | NO:10) |

Using the GAS:SEAP/Neo vector produced in Example 13, EGR1 amplified product can then be inserted into this vector. Linearize the GAS:SEAP/Neo vector using restriction enzymes XhoI/HindIII, removing the GAS/SV40 stuffer. Restrict the EGR1 amplified product with these same enzymes. Ligate the vector and the EGR1 promoter.

To prepare 96 well-plates for cell culture, two mls of a coating solution (1:30 dilution of collagen type I (Upstate Biotech Inc. Cat#08-115) in 30% ethanol (filter sterilized)) is added per one 10 cm plate or 50 ml per well of the 96-well plate, and allowed to air dry for 2 hr.

PCI2 cells are routinely grown in RPMI-1640 medium (Bio Whittaker) containing 10% horse serum (JRH BIOSCIENCES, Cat. # 12449-78P), 5% heat-inactivated fetal bovine serum (FBS) supplemented with 100 units/ml penicillin and 100 ug/ml streptomycin on a precoated 10 cm tissue culture dish. One to four split is done every three to four days. Cells are removed from the plates by scraping and resuspended with pipetting up and down for more than 15 times.

Transfect the EGR/SEAP/Neo construct into PCI2 using the Lipofectamine protocol described in Example 12. EGR-SEAP/PCI2 stable cells are obtained by growing the cells in 300 ug/ml G418. The G418-free medium is used for routine growth but every one to two months, the cells should be re-grown in 300 ug/ml G418 for couple of passages.

To assay for neuronal activity, a 10 cm plate with cells around 70 to 80% confluent is screened by removing the old medium. Wash the cells once with PBS (Phosphate buffered saline). Then starve the cells in low serum medium (RPMI-1640 containing 1% horse serum and 0.5% FBS with antibiotics) overnight.

The next morning, remove the medium and wash the cells with PBS. Scrape off the cells from the plate, suspend the cells well in 2 ml low serum medium. Count the cell number and add more low serum medium to reach final cell density as 5×10⁵ cells/ml.

Add 200 ul of the cell suspension to each well of 96-well plate (equivalent to 1×10⁵ cells/well). Add 50 ul supernatant produced by Example 12, 37° C. for 48 to 72 hr. As a positive control, a growth factor known to activate PCI2 cells through EGR can be used, such as 50 ng/ul of Neuronal Growth Factor (NGF). Over fifty-fold induction of SEAP is typically seen in the positive control wells. SEAP assay the supernatant according to Example 18.

### Example 17

### High-Throughput Screening Assay for T-cell Activity

NF-κB (Nuclear Factor κB) is a transcription factor activated by a wide variety of agents including the inflammatory cytokines IL-1 and TNF, CD30 and CD40, lymphotoxin-alpha and lymphotoxin-beta, by exposure to LPS or thrombin, and by expression of certain viral gene products. As a transcription factor, NF-κB regulates the expression of genes involved in immune cell activation, control of apoptosis (NF-κB appears to shield cells from apoptosis), B and T-cell development, anti-viral and antimicrobial responses, and multiple stress responses.

In non-stimulated conditions, NF-κB is retained in the cytoplasm with I-κB (Inhibitor κB). However, upon stimulation, I-κB is phosphorylated and degraded, causing NF-κB to shuttle to the nucleus, thereby activating transcription of target genes. Target genes activated by NF-κB include IL-2, IL-6, GM-CSF, ICAM-1 and class 1 MHC.

Due to its central role and ability to respond to a range of stimuli, reporter constructs utilizing the NF-κB promoter element are used to screen the supernatants produced in Example 12. Activators or inhibitors of NF-κB would be useful in treating diseases. For example, inhibitors of NF-κB could be used to treat those diseases related to the acute or chronic activation of NF-κB, such as rheumatoid arthritis.

To construct a vector containing the NF-κB promoter element, a PCR based strategy is employed. The upstream primer contains four tandem copies of the NF-κB binding site (GGGGACTTTCCC) (SEQ ID NO:11), 18 bp of sequence complementary to the 5′ end of the SV40 early promoter sequence, and is flanked with an XhoI site:

| | |
|---|---|
| 5′:GCGGCCTCGAGGGGACTTTCCCGGGGACTTTCCGGGGACTTTCCGGGACTTTCC | (SEQ ID NO:12) |
| ATCCTGCCATCTCAATTAG:3′ | |

The downstream primer is complementary to the 3′ end of the SV40 promoter and is flanked with a Hind III site: 5′:GCGGCAAGCTTTTTGCAAAGCCTAGGC:3′ (SEQ ID NO:7)

PCR amplification is performed using the SV40 promoter template present in the pB-gal:promoter plasmid obtained from Clontech. The resulting PCR fragment is digested with XhoI and Hind III and subcloned into BLSK2-. (Stratagene) Sequencing with the T7 and T3 primers confirms the insert contains the following sequence:

| | |
|---|---|
| 5′:CTCGAGGGGACTTTCCCGGGGACTTTCCGGGGACTTTCCGGGACTTTCCATCTG | (SEQ ID NO:13) |
| CCATCTCAATTAGTCAGCAACCATAGTCCCGCCCCTAACTCCGCCCATCCCGCCC | |
| CTAACTCCGCCCAGTTCCGCCCATTCTCCGCCCCATGGCTGACTAATTTTTTTTAT | |
| TTATGCAGAGGCCGAGGCCGCCTCGGCCTCTGAGCTATTCCAGAAGTAGTGAGGA | |
| GGCTTTTTTGGAGGCCTAGGCTTTTGCAAAAAGCTT:3′ | |

Next, replace the SV40 minimal promoter element present in the pSEAP2-promoter plasmid (Clontech) with this NF-κB/SV40 fragment using XhoI and HindIII. However, this vector does not contain a neomycin resistance gene, and therefore, is not preferred for mammalian expression systems.

In order to generate stable mammalian cell lines, the NF-κB/SV40/SEAP cassette is removed from the above NF-κB/SEAP vector using restriction enzymes SalI and NotI, and inserted into a vector containing neomycin resistance. Particularly, the NF-κB/SV40/SEAP cassette was inserted into pGFP-1 (Clontech), replacing the GFP gene, after restricting pGFP-1 with SalI and NotI.

Once NF-κB/SV40/SEAP/Neo vector is created, stable Jurkat T-cells are created and maintained according to the protocol described in Example 14. Similarly, the method for assaying supernatants with these stable Jurkat T-cells is also described in Example 14. As a positive control, exogenous TNF alpha (0.1, 1, 10 ng) is added to wells H9, H10, and H11, with a 5-10 fold activation typically observed.

### Example 18

### Assay for SEAP Activity

As a reporter molecule for the assays described in Examples 14-17, SEAP activity is assayed using the Tropix Phospho-light Kit (Cat. BP-400) according to the following general procedure. The Tropix Phospho-light Kit supplies the Dilution, Assay, and Reaction Buffers used below.

Prime a dispenser with the 2.5× Dilution Buffer and dispense 15 μl of 2.5× dilution buffer into Optiplates containing 35 μl of a supernatant. Seal the plates with a plastic sealer and incubate at 65° C. for 30 min. Separate the Optiplates to avoid uneven heating.

Cool the samples to room temperature for 15 minutes. Empty the dispenser and prime with the Assay Buffer. Add 50 μl Assay Buffer and incubate at room temperature 5 min. Empty the dispenser and prime with the Reaction Buffer (see the table below). Add 50 μl Reaction Buffer and incubate at room temperature for 20 minutes. Since the intensity of the chemiluminescent signal is time dependent, and it takes about 10 minutes to read 5 plates on luminometer, one should treat 5 plates at each time and start the second set 10 minutes later.

Read the relative light unit in the luminometer. Set H12 as blank, and print the results. An increase in chemiluminescence indicates reporter activity.

Reaction Buffer Formulation:

| # of plates | Rxn buffer diluent (ml) | CSPD (ml) |
|---|---|---|
| 10 | 60 | 3 |
| 11 | 65 | 3.25 |
| 12 | 70 | 3.5 |
| 13 | 75 | 3.75 |
| 14 | 80 | 4 |
| 15 | 85 | 4.25 |
| 16 | 90 | 4.5 |
| 17 | 95 | 4.75 |
| 18 | 100 | 5 |
| 19 | 105 | 5.25 |
| 20 | 110 | 5.5 |
| 21 | 115 | 5.75 |
| 22 | 120 | 6 |
| 23 | 125 | 6.25 |
| 24 | 130 | 6.5 |
| 25 | 135 | 6.75 |
| 26 | 140 | 7 |
| 27 | 145 | 7.25 |
| 28 | 150 | 7.5 |
| 29 | 155 | 7.75 |
| 30 | 160 | 8 |
| 31 | 165 | 8.25 |
| 32 | 170 | 8.5 |
| 33 | 175 | 8.75 |
| 34 | 180 | 9 |
| 35 | 185 | 9.25 |
| 36 | 190 | 9.5 |
| 37 | 195 | 9.75 |
| 38 | 200 | 10 |
| 39 | 205 | 10.25 |
| 40 | 210 | 10.5 |
| 41 | 215 | 10.75 |
| 42 | 220 | 11 |
| 43 | 225 | 11.25 |
| 44 | 230 | 11.5 |
| 45 | 235 | 11.75 |
| 46 | 240 | 12 |
| 47 | 245 | 12.25 |
| 48 | 250 | 12.5 |
| 49 | 255 | 12.75 |
| 50 | 260 | 13 |

### Example 19

### High-Throughput Screening Assay Identifying Changes in Small Molecule Concentration and Membrane Permeability

Binding of a ligand to a receptor is known to alter intracellular levels of small molecules, such as calcium, potassium, sodium, and pH, as well as alter membrane potential. These alterations can be measured in an assay to identify supernatants which bind to receptors of particular cell. Although the following protocol describes an assay for calcium, this protocol can easily be modified to detect changes in potassium, sodium, pH, membrane potential, or any other small molecule which is detectable by a fluorescent probe.

The following assay uses Fluorometric Imaging Plate Reader (“FLIPR”) to measure changes in fluorescent molecules (Molecular Probes) that bind small molecules. Clearly, any fluorescent molecule detecting a small molecule can be used instead of the calcium fluorescent molecule, fluo-3, used here.

For adherent cells, seed the cells at 10,000-20,000 cells/well in a Co-star black 96-well plate with clear bottom. The plate is incubated in a CO₂ incubator for 20 hours. The adherent cells are washed two times in Biotek washer with 200 ul of HBSS (Hank's Balanced Salt Solution) leaving 100 ul of buffer after the final wash.

A stock solution of 1 mg/ml fluo-3 is made in 10% pluronic acid DMSO. To load the cells with fluo-3, 50 ul of 12 μg/ml fluo-3 is added to each well. The plate is incubated at 37° C. in a CO₂ incubator for 60 min. The plate is washed four times in the Biotek washer with HBSS leaving 100 μl of buffer.

For non-adherent cells, the cells are spun down from culture media. Cells are resuspended to 2-5×10⁶ cells/ml with HBSS in a 50-ml conical tube. 4 μl of 1 mg/ml fluo-3 solution in 10% pluronic acid DMSO is added to each ml of cell suspension. The tube is then placed in a 37° C. water bath for 30-60 min. The cells are washed twice with HBSS, resuspended to 1×10⁶ cells/ml, and dispensed into a microplate, 100 μl/well. The plate is centrifuged at 1000 rpm for 5 min. The plate is then washed once in Denley CellWash with 200 μl, followed by an aspiration step to 100 μl final volume.

For a non-cell based assay, each well contains a fluorescent molecule, such as fluo-3. The supernatant is added to the well, and a change in fluorescence is detected.

To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 μl. Increased emission at 530 nm indicates an extracellular signaling event caused by the a molecule, either PDEF or a molecule induced by PDEF, which has resulted in an increase in the intracellular Ca⁺⁺ concentration.

### Example 20

### High-Throughput Screening Assay Identifying Tyrosine Kinase Activity

The Protein Tyrosine Kinases (PTK) represent a diverse group of transmembrane and cytoplasmic kinases. Within the Receptor Protein Tyrosine Kinase (RPTK) group are receptors for a range of mitogenic and metabolic growth factors including the PDGF, FGF, EGF, NGF, HGF and Insulin receptor subfamilies. In addition there are a large family of RPTKs for which the corresponding ligand is unknown. Ligands for RPTKs include mainly secreted small proteins, but also membrane-bound and extracellular matrix proteins.

Activation of RPTK by ligands involves ligand-mediated receptor dimerization, resulting in transphosphorylation of the receptor subunits and activation of the cytoplasmic tyrosine kinases. The cytoplasmic tyrosine kinases include receptor associated tyrosine kinases of the src-family (e.g., src, yes, lck, lyn, fyn) and non-receptor linked and cytosolic protein tyrosine kinases, such as the Jak family, members of which mediate signal transduction triggered by the cytokine superfamily of receptors (e.g., the Interleukins, Interferons, GM-CSF, and Leptin).

Because of the wide range of known factors capable of stimulating tyrosine kinase activity, identifying whether PDEF or a molecule induced by PDEF is capable of activating tyrosine kinase signal transduction pathways is of interest. Therefore, the following protocol is designed to identify such molecules capable of activating the tyrosine kinase signal transduction pathways.

Seed target cells (e.g., primary keratinocytes) at a density of approximately 25,000 cells per well in a 96 well Loprodyne Silent Screen Plates purchased from Nalge Nunc (Naperville, Ill.). The plates are sterilized with two 30 minute rinses with 100% ethanol, rinsed with water and dried overnight. Some plates are coated for 2 hr with 100 ml of cell culture grade type I collagen (50 mg/ml), gelatin (2%) or polylysine (50 mg/ml), al of which can be purchased from Sigma Chemicals (St. Louis, Mo.) or 10% Matrigel purchased from Becton Dickinson (Bedford, Mass.), or calf serum, rinsed with PBS and stored at 4° C. Cell growth on these plates is assayed by seeding 5,000 cells/well in growth medium and indirect quantitation of cell number through use of alamarBlue as described by the manufacturer Alamar Biosciences, Inc. (Sacramento, Calif.) after 48 hr. Falcon plate covers #3071 from Becton Dickinson (Bedford, Mass.) are used to cover the Loprodyne Silent Screen Plates. Falcon Microtest III cell culture plates can also be used in some proliferation experiments.

To prepare extracts, A431 cells are seeded onto the nylon membranes of Loprodyne plates (20,000/200 ml/well) and cultured overnight in complete medium. Cells are quiesced by incubation in serum-free basal medium for 24 hr. After 5-20 minutes treatment with EGF (60 ng/ml) or 50 ul of the supernatant produced in Example 12, the medium was removed and 100 ml of extraction buffer ((20 mM HEPES pH 7.5, 0.15 M NaCl, 1% Triton X-100, 0.1% SDS, 2 mM Na3VO4, 2 mM Na4P207 and a cocktail of protease inhibitors (# 1836170) obtained from Boehringer Mannheim (Indianapolis, Ind.) is added to each well and the plate is shaken on a rotating shaker for 5 minutes at 4° C. The plate is then placed in a vacuum transfer manifold and the extract filtered through the 0.45 mm membrane bottoms of each well using house vacuum. Extracts are collected in a 96-well catch/assay plate in the bottom of the vacuum manifold and immediately placed on ice. To obtain extracts clarified by centrifugation, the content of each well, after detergent solubilization for 5 minutes, is removed and centrifuged for 15 minutes at 4° C. at 16,000×g.

Test the filtered extracts for levels of tyrosine kinase activity. Although many methods of detecting tyrosine kinase activity are known, one method is described here.

Generally, the tyrosine kinase activity of a supernatant is evaluated by determining its ability to phosphorylate a tyrosine residue on a specific substrate (a biotinylated peptide). Biotinylated peptides that can be used for this purpose include PSK1 (corresponding to amino acids 6-20 of the cell division kinase cdc2-p34) and PSK2 (corresponding to amino acids 1-17 of gastrin). Both peptides are substrates for a range of tyrosine kinases and are available from Boehringer Mannheim.

The tyrosine kinase reaction is set up by adding the following components in order. First, add 10 μl of 5 uM Biotinylated Peptide, then 10 ul ATP/Mg₂₊ (5 mM ATP/50 mM MgCl₂), then 10 μl of 5× Assay Buffer (40 mM imidazole hydrochloride, pH7.3, 40 mM beta-glycerophosphate, 1 mM EGTA, 100 mM MgCl₂, 5 mM MnCl₂, 0.5 mg/ml BSA), then 5 μl of Sodium Vanadate(1 mM), and then 5 μl of water. Mix the components gently and preincubate the reaction mix at 30° C. for 2 min. Initial the reaction by adding 10 μl of the control enzyme or the filtered supernatant.

The tyrosine kinase assay reaction is then terminated by adding 10 μl of 120 mm EDTA and place the reactions on ice.

Tyrosine kinase activity is determined by transferring 50 μl aliquot of reaction mixture to a microtiter plate (MTP) module and incubating at 37° C. for 20 min. This allows the streptavadin coated 96 well plate to associate with the biotinylated peptide. Wash the MTP module with 300 μl/well of PBS four times. Next add 75 μl of anti-phospotyrosine antibody conjugated to horse radish peroxidase(anti-P-Tyr-POD(0.5 u/ml)) to each well and incubate at 37° C. for one hour. Wash the well as above.

Next add 100 μl of peroxidase substrate solution (Boehringer Mannheim) and incubate at room temperature for at least 5 mins (up to 30 min). Measure the absorbance of the sample at 405 nm by using ELISA reader. The level of bound peroxidase activity is quantitated using an ELISA reader and reflects the level of tyrosine kinase activity.

### Example 21

### High-Throughput Screening Assay Identifying Phosphorylation Activity

As a potential alternative and/or compliment to the assay of protein tyrosine kinase activity described in Example 20, an assay which detects activation (phosphorylation) of major intracellular signal transduction intermediates can also be used. For example, as described below one particular assay can detect tyrosine phosphorylation of the Erk-1 and Erk-2 kinases. However, phosphorylation of other molecules, such as Raf, JNK, p38 MAP, Map kinase kinase (MEK), MEK kinase, Src, Muscle specific kinase (MuSK), IRAK, Tec, and Janus, as well as any other phosphoserine, phosphotyrosine, or phosphothreonine molecule, can be detected by substituting these molecules for Erk-1 or Erk-2 in the following assay.

Specifically, assay plates are made by coating the wells of a 96-well ELISA plate with 0.1 ml of protein G (1 μg/ml) for 2 hr at room temp, (RT). The plates are then rinsed with PBS and blocked with 3% BSA/PBS for 1 hr at RT. The protein G plates are then treated with 2 commercial monoclonal antibodies (100 ng/well) against Erk-1 and Erk-2 (1 hr at RT) (Santa Cruz Biotechnology). (To detect other molecules, this step can easily be modified by substituting a monoclonal antibody detecting any of the above described molecules.) After 3-5 rinses with PBS, the plates are stored at 4° C. until use.

A431 cells are seeded at 20,000/well in a 96-well Loprodyne filterplate and cultured overnight in growth medium. The cells are then starved for 48 hr in basal medium (DMEM) and then treated with EGF (6 ng/well) or 50 μl of the supernatants obtained in Example 12 for 5-20 minutes. The cells are then solubilized and extracts filtered directly into the assay plate.

After incubation with the extract for 1 hr at RT, the wells are again rinsed. As a positive control, a commercial preparation of MAP kinase (10 ng/well) is used in place of A431 extract. Plates are then treated with a commercial polyclonal (rabbit) antibody (1 μg/ml) which specifically recognizes the phosphorylated epitope of the Erk-1 and Erk-2 kinases (1 hr at RT). This antibody is biotinylated by standard procedures. The bound polyclonal antibody is then quantitated by successive incubations with Europium-streptavidin and Europium fluorescence enhancing reagent in the Wallac DELFIA instrument (time-resolved fluorescence). An increased fluorescent signal over background indicates a phosphorylation by PDEF or a molecule induced by PDEF.

### Example 22

### Method of Determining Alterations in the PDEF Gene

RNA isolated from entire families or individual patients presenting with a phenotype of interest (such as a disease) is be isolated. cDNA is then generated from these RNA samples using protocols known in the art. (See, Sambrook.) The cDNA is then used as a template for PCR, employing primers surrounding regions of interest in SEQ ID NO:1. Suggested PCR conditions consist of 35 cycles at 95° C. for 30 seconds; 60-120 seconds at 52-58° C.; and 60-120 seconds at 70° C., using buffer solutions described in Sidransky, D., et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons of PDEF is also determined and genomic PCR products analyzed to confirm the results. PCR products harboring suspected mutations in PDEF is then cloned and sequenced to validate the results of the direct sequencing.

PCR products of PDEF are cloned into T-tailed vectors as described in Holton, T. A. and Graham, M. W., Nucleic Acids Research, 19:1156 (1991) and sequenced with T7 polymerase (United States Biochemical). Affected individuals are identified by mutations in PDEF not present in unaffected individuals.

Genomic rearrangements are also observed as a method of determining alterations in the PDEF gene. Genomic clones isolated according to Example 2 are nick-translated with digoxigenindeoxy-uridine 5′-triphosphate (Boehringer Manheim), and FISH performed as described in Johnson, Cg. et al., Methods Cell Biol. 35:73-99(1991). Hybridization with the labeled probe is carried out using a vast excess of human cot-I DNA for specific hybridization to the PDEF genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson, Cv. et al., Genet. Anal. Tech. Appl., 8:75 (1991).) Image collection, analysis and chromosomal fractional length measurements are performed using the ISee Graphical Program System. (Inovision Corporation, Durham, N.C.) Chromosome alterations of the genomic region of PDEF (hybridized by the probe) are identified as insertions, deletions, and translocations. These PDEF alterations are used as a diagnostic marker for an associated disease.

### Example 23

### Method of Detecting Abnormal Levels of PDEF in a Biological Sample

PDEF polypeptides can be detected in a biological sample, and if an increased or decreased level of PDEF is detected, this polypeptide is a marker for a particular phenotype. Methods of detection are numerous, and thus, it is understood that one skilled in the art can modify the following assay to fit their particular needs.

For example, antibody-sandwich ELISAs are used to detect PDEF in a sample, preferably a biological sample. Wells of a microtiter plate are coated with specific antibodies to PDEF, at a final concentration of 0.2 to 10 μg/ml. The antibodies are either monoclonal or polyclonal and are produced by the method described in Example 11. The wells are blocked so that non-specific binding of PDEF to the well is reduced.

The coated wells are then incubated for >2 hours at RT with a sample containing PDEF. Preferably, serial dilutions of the sample should be used to validate results. The plates are then washed three times with deionized or distilled water to remove unbounded PDEF.

Next, 50 μl of specific antibody-alkaline phosphatase conjugate, at a concentration of 25-400 ng, is added and incubated for 2 hours at room temperature. The plates are again washed three times with deionized or distilled water to remove unbounded conjugate.

Add 75 μl of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot PDEF polypeptide concentration on the X-axis (log scale) and fluorescence or absorbance of the Y-axis (linear scale). Interpolate the concentration of the PDEF in the sample using the standard curve.

### Example 24

### Formulating a Polypeptide

The PDEF composition will be formulated and dosed in a fashion consistent with good medical practice, taking into account the clinical condition of the individual patient (especially the side effects of treatment with the PDEF polypeptide alone), the site of delivery, the method of administration, the scheduling of administration, and other factors known to practitioners. The “effective amount” for purposes herein is thus determined by such considerations.

As a general proposition, the total pharmaceutically effective amount of PDEF administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, PDEF is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing PDEF are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrastemal, subcutaneous and intraarticular injection and infusion.

PDEF is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langeret al.) or poly-D-(−)-3-hydroxybutyric acid (EP 133,988). Sustained-release compositions also include liposomally entrapped PDEF polypeptides. Liposomes containing the PDEF are prepared by methods known per se: DE 3,218,121; Epstein et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal secreted polypeptide therapy.

For parenteral administration, in one embodiment, PDEF is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting PDEF uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

PDEF is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

PDEF used for therapeutic administration can be sterile. Sterility is readily accomplished by filtration through sterile filtration membranes (e.g., 0.2 micron membranes). Therapeutic polypeptide compositions generally are placed into a container having a sterile access port, for example, an intravenous solution bag or vial having a stopper pierceable by a hypodermic injection needle.

PDEF polypeptides ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous PDEF polypeptide solution, and the resulting mixture is lyophilized. The infusion solution is prepared by reconstituting the lyophilized PDEF polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, PDEF may be employed in conjunction with other therapeutic compounds.

### Example 25

### Method of Treating Decreased Levels of PDEF

The present invention relates to a method for treating an individual in need of a decreased level of PDEF activity in the body comprising, administering to such an individual a composition comprising a therapeutically effective amount of PDEF antagonist. Preferred antagonists for use in the present invention are PDEF-specific antibodies.

Moreover, it will be appreciated that conditions caused by a decrease in the standard or normal expression level of PDEF in an individual can be treated by administering PDEF, preferably in the soluble form. Thus, the invention also provides a method of treatment of an individual in need of an increased level of PDEF polypeptide comprising administering to such an individual a pharmaceutical composition comprising an amount of PDEF to increase the activity level of PDEF in such an individual.

For example, a patient with decreased levels of PDEF polypeptide receives a daily dose 0.1-100 μg/kg of the polypeptide for six consecutive days. Preferably, the polypeptide is in the soluble form. The exact details of the dosing scheme, based on administration and formulation, are provided in Example 24.

### Example 26

### Method of Treating Increased Levels of PDEF

The present invention also relates to a method for treating an individual in need of an increased level of PDEF activity in the body comprising administering to such an individual a composition comprising a therapeutically effective amount of PDEF or an agonist thereof.

Antisense technology is used to inhibit production of PDEF. This technology is one example of a method of decreasing levels of PDEF polypeptide, preferably a soluble form, due to a variety of etiologies, such as cancer.

For example, a patient diagnosed with abnormally increased levels of PDEF is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided in Example 24.

### Example 27

### Method of Treatment Using Gene Therapy—Ex Vivo

One method of gene therapy transplants fibroblasts, which are capable of expressing PDEF polypeptides, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37° C. for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al., DNA, 7:219-25 (1988)), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding PDEF can be amplified using PCR primers which correspond to the 5′ and 3′ end sequences respectively as set forth in Example 1. Preferably, the 5′ primer contains an EcoRI site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector contains properly inserted PDEF.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the PDEF gene is then added to the media and the packaging cells transduced with the vector. The packaging cells now produce infectious viral particles containing the PDEF gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a subconfluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether PDEF protein is produced.

The engineered fibroblasts are then transplanted onto the host, either alone or after having been grown to confluence on cytodex 3 microcarrier beads.

### Example 28

### Method of Treatment Using Gene Therapy—In Vivo

Another aspect of the present invention is using in vivo gene therapy methods to treat disorders, diseases and conditions. The gene therapy method relates to the introduction of naked nucleic acid (DNA, RNA, and antisense DNA or RNA) PDEF sequences into an animal to increase or decrease the expression of the PDEF polypeptide. The PDEF polynucleotide may be operatively linked to a promoter or any other genetic elements necessary for the expression of the PDEF polypeptide by the target tissue. Such gene therapy and delivery techniques and methods are known in the art, see, for example, WO90/11092, WO98/11779; U.S. Pat. Nos. 5693622, 5705151, 5580859; Tabata H. et al. (1997) Cardiovasc. Res. 35(3):470-479, Chao J et al. (1997) Pharmacol. Res. 35(6):517-522, Wolff J. A. (1997) Neuromuscul. Disord. 7(5):314-318, Schwartz B. et al. (1996) Gene Ther. 3(5):405-411, Tsurumi Y. et al. (1996) Circulation 94(12):3281-3290 (incorporated herein by reference).

The PDEF polynucleotide constructs maybe delivered by any method that delivers injectable materials to the cells of an animal, such as, injection into the interstitial space of tissues (heart, muscle, skin, lung, liver, intestine and the like). The PDEF polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the PDEF polynucleotides may also be delivered in liposome formulations (such as those taught in Felgner P. L. et al. (1995) Ann. NY Acad. Sci. 772:126-139 and Abdallah B. et al. (1995) Biol. Cell 85(1):1-7) which can be prepared by methods well known to those skilled in the art.

The PDEF polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The PDEF polynucleotide construct can be delivered to the interstitial space of tissues within an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked PDEF polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked PDEF polynucleotide constructs can be delivered to arteries during angioplasty by the catheter used in the procedure.

The dose response effects of injected PDEF polynucleotide in muscle in vivo is determined as follows. Suitable PDEF template DNA for production of mRNA coding for PDEF polypeptide is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The PDEF template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for PDEF protein expression. A time course for PDEF protein expression may be done in a similar fashion except that quadriceps from different mice are harvested at different times. Persistence of PDEF DNA in muscle following injection maybe determined by Southern blot analysis after preparing total cellular DNA and HIRT supernatants from injected and control mice. The results of the above experimentation in mice can be use to extrapolate proper dosages and other treatment parameters in humans and other animals using PDEF naked DNA.

### Example 29

### PDEF Expression During Androgen Stimulation

Considering the sensitivity of prostate epithelial cells to androgen stimulation, in addition to, the high expression of PDEF in prostate epithelial cells, it would be of interest to know whether PDEF may be involved in the androgen response. Human prostate cancer cells (LNCaP) were starved in a specialized growth medium devoid of any steroids prior to androgen stimulation. The addition of androgen initiates a program of gene expression which progresses over the period of more than 24 h through various stages of up- and down-regulation of distinct prostate marker genes, such as PSA.

To evaluate PDEF expression during androgen induction of LNCAP cells, RT/PCR with mRNA derived from starved LNCaP cells, and from LNCaP cells of different time points after induction with androgen, was performed. To test for RNA quality and to roughly compare relative levels of expression, each RNA sample was analyzed by RT/PCR for GAPDH expression. Conditions for RT/PCR include: cDNAs were generated from 1 μg mRNA isolated from different cells or tissues using oligo dT12-18 priming (Gibco BRL Grand Island, N.Y. USA) and M-MLV reverse transcriptase (Gibco BRL) in deoxyribonuclease I (Gibco BRL) treated samples. Each PCR used equivalent amounts of (0.1 ng) cDNA, 4 ng/μl of each primer, 0.25 units of Taq polymerase (Promega, Madison, Wis. USA), 150 μM of each dNTP, 3 mM of MgCl₂, reaction buffer and water to a final volume of 25 μl and were covered with mineral oil. The sequences of the primers for GAPDH were: sense: 5′-CAAAGTTGTCATGGATGACC-3′ (SEQ ID NO:14) antisense: 5′-CCATGGAGAAGGCTGGGG-3′ (SEQ ID NO:15) with an expected amplification product of 200 bp. RT/PCR amplifications were carried out using a Perkin-Elmer Cetus thermal cycler 480 as follows: 20-30 cycles of 1 min at 94° C., 1 min at 56° C. and 1 min at 72° C. followed by 15 min at 72° C. Lower numbers of cycles were used to verify linearity of the amplification signal. The amplification product was analyzed on a 2% agarose gel.

Using equal amounts of cDNA from each time point for PCR amplification very similar levels of GAPDH amplification products were obtained for all samples, even when using different numbers of amplification cycles. PDEF expression was already detected in unstimulated LNCaP cells. However, twelve hours after stimulation enhanced expression of PDEF was clearly visible. These results suggest that PDEF expression is upregulated during androgen stimulation of prostate cancer cells twelve hours after stimulation.

To determine whether expression of another Ets factor ESE-1 is inducible as well, RT/PCR using ESE-1 specific primers was performed. In contrast to PDEF, expression of ESE-1 did not vary significantly during androgen stimulation indicating that PDEF is specifically upregulated during androgen stimulation.

### Example 30

### PDEF Specifically Enhances Transcription of the PSA Gene, Unlike other Ets Factors

In assessing whether PDEF acts as a repressor or enhancer of transcription, and to evaluate the possibility that the prostate-specific prostate specific antigen (PSA) gene is a target for PDEF, full length PDEF inserted into a eukaryotic expression vector (pCI/PDEF) was co-transfected into PDEF negative CV-1 cells, together with a pGL2 reporter gene construct containing the luciferase gene under the control of the PSA promoter. PSA encodes a secreted protein of significant clinical value due to its use as a diagnostic marker for human prostate cancer. In addition, expression vectors encoding a variety of other Ets factors were co-transfected to compare their activity to PDEF.

Co-transfections of 3×10⁵ CV-1 cells were carried out with 2 _g reporter gene construct DNA and 3 μg expression vector DNA using 12.5 μl lipofectamine (Gibco-BRL) as described. The cells were either harvested 16 hours after transfection or starved in androgen-free medium containing charcoal stripped FCS for 24 h and then induced with androgen for 24 h and assayed for luciferase activity. Transfections for every construct were performed independently in duplicate and repeated 3 to 4 times with two different plasmid preparations with similar results. Cotransfection of a second plasmid for determination of transfection efficiency was omitted because potential artifacts with this technique have been reported and because many commonly used viral promoters contain potential binding sites for Ets factors.

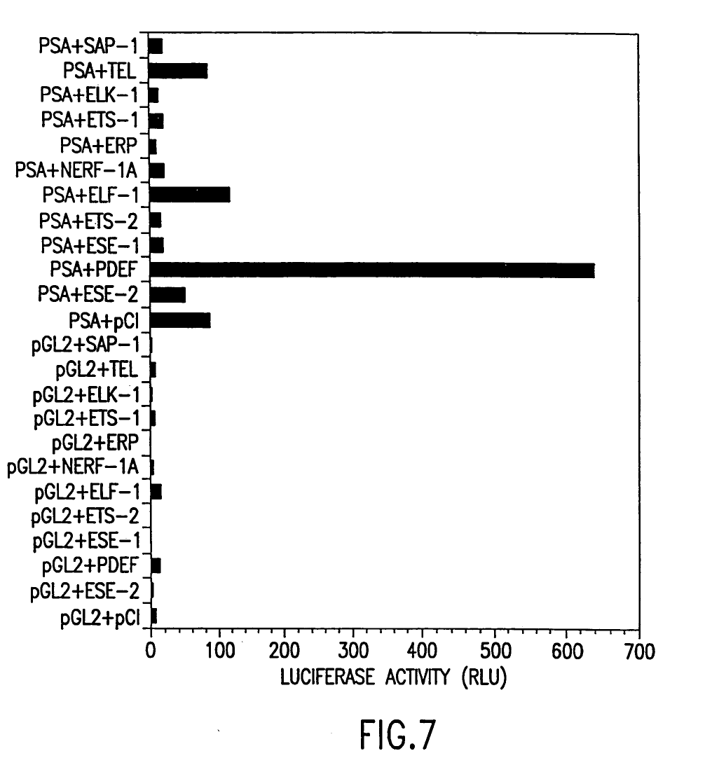

Co-transfection with pCI/PDEF resulted in a ˜7-fold transcriptional stimulation of the PSA promoter compared to the parental pCI vector. (See FIG. 7.) In contrast, none of the other Ets factors had any significant effect on PSA promoter activity, even though some of them such as ESE-1 appeared to repress rather than enhance the PSA promoter. Thus, the PSA gene can be activated by PDEF demonstrating the utility of PDEF as a positive regulator of transcription which implies that PSA may indeed be a relevant prostate-specific target for PDEF. Conditions for co-transfections also included: Co-transfections of 3×10⁵ CV-1 cells were carried out with 3 μg reporter gene construct DNA and 1 μg expression vector DNA using 12.5 μl lipofectamine (Gibco-BRL). Cells were washed with serum free DMEM. 1.6 ml serum free DMEM was added per well. Liposomes were incubated with the DNA in 200 μl serum free DMEM for 15 min at room temperature and then incubated with the cells for 4 h at 37° C. 2 ml DMEM containing 20% fetal calf serum (FCS) was added, the cells wer harvested 16 hours after transfection and assayed for luciferase activity as described. Transfections for every construct were performed independently in duplicates and repeated 2-5 times with at least two different plasmid preparations with similar results (Oettgen, P, Akbarali, Y, Boltax, J, Best, J, Kunsch, C and Libermann, Ta (1996), Mol. Cell. Biol. 16: 5091-5106).

### Example 31

### PDEF Interacts with the Androgen Receptor and Synergistically Enhances Transcription of the PSA Gene

In addition to the Ets sites, various other regulatory elements are present in the PSA promoter including an androgen receptor binding site which has been shown to be critical for androgen inducibility of the PSA gene. A characteristic feature of all Ets factors is their ability to interact with other transcription factors, suggesting that interactions of PDEF with factors binding to regulatory elements within the PSA promoter may be an important mechanism of transcriptional control.

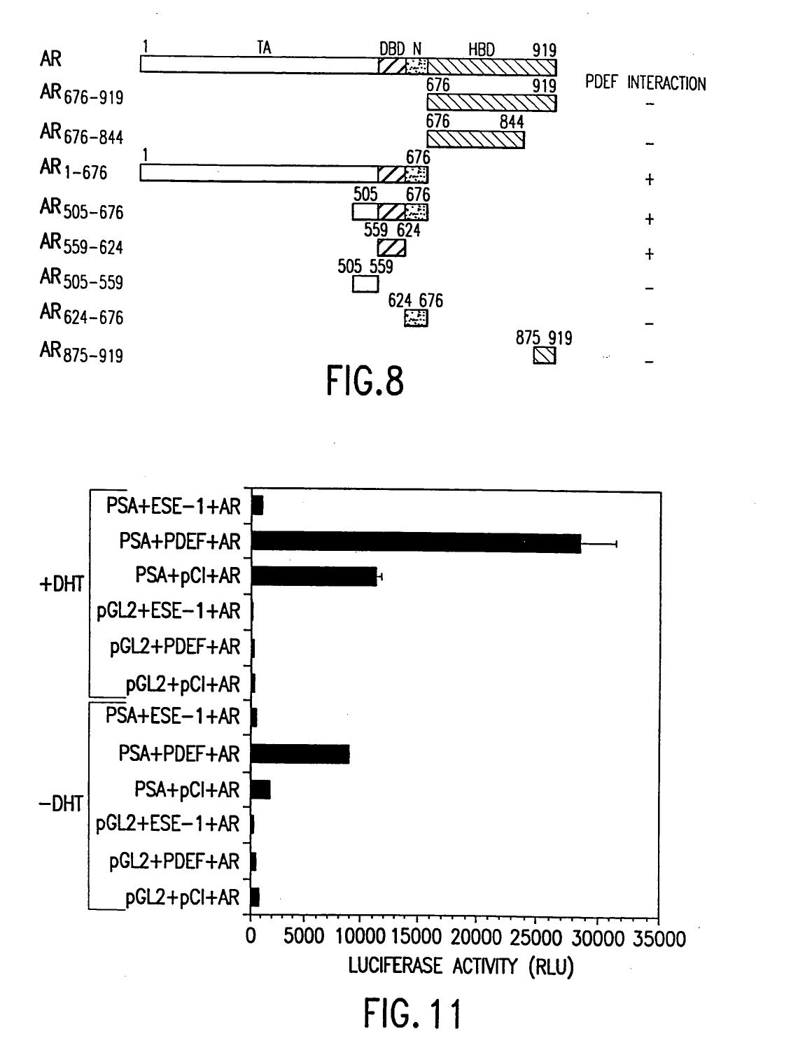

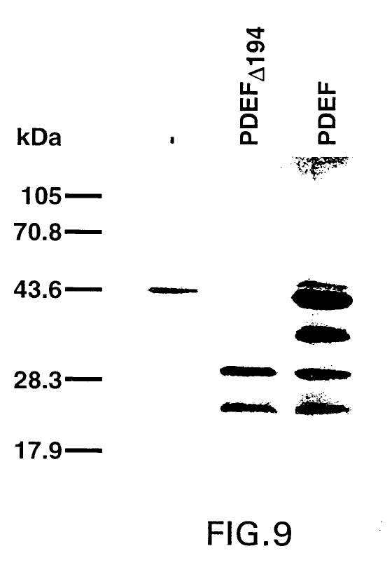

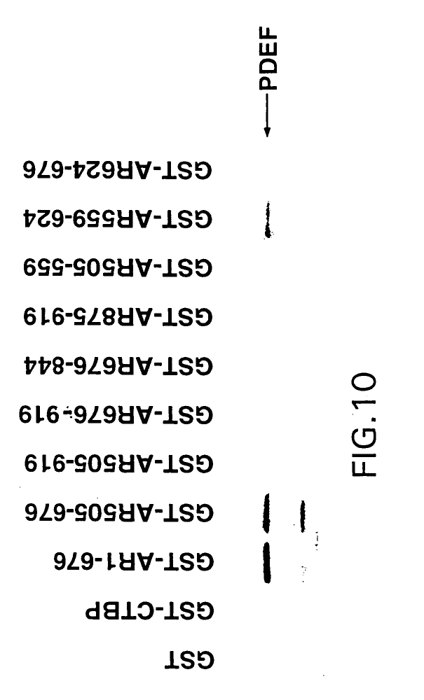

To explore the possibility that PDEF might interact with the androgen receptor, GST (Glutathione-S-Transferase) pull down experiments were performed. Different domains of the full-length PDEF gene were fused to the GST coding region and expressed in bacteria as fusion proteins. (See FIG. 8.) PDEF was in vitro transcribed and translated into protein in a reticulocyte lysate revealing as the major product a protein of the expected molecular weight. (See FIG. 9.) Minor amounts of additional faster migrating (lower molecular weight) proteins were present due to either partial proteolysis, internal translation initiation, or premature translational termination. Plasmid constructs for in vitro translation were constructed by inserting the full length PDEF cDNA, encoding the entire open reading frame, downstream of the T7 promoter into TA cloning vector, pCR2 (Invitrogen). Coupled in vitro transcription/in vitro translation reactions were performed (Promega). Affinity-purified GST-androgen receptor fragments were incubated with in vitro translated 35S-methionine labeled PDEF, washed, and analyzed by SDS-PAGE (Sodium Dodecyl-Sulfate Poly-Acrylamide Gel Electrophoresis). (See FIG. 10.)

Conditions for in vitro translation included: Full length PDEF cDNA encoding the entire open reading frame was inserted into the TA cloning vector (Invitrogen) with the T7 promoter upstream of the initiator methionine. Coupled in vitro transcription/in vitro translation reactions were performed with 1 _g plasmid using the TNT reticulocyte lysate kit (Promega) and T7 RNA polymerase as recommended by the manufacturer in the presence of either (³⁵S)-methionine (NEN) or cold methionine. A portion of the labeled in vitro translation product was analyzed on a 10% sodium dodecyl sulfate-polyacrylamide gel (SDS-PAGE).

PDEF was brought down specifically by any GST-androgen receptor fusion protein containing the DNA binding domain, but not by GST alone or by other domains of the androgen receptor.

To evaluate whether the physical interaction between PDEF and androgen receptor correlates with functional interaction, PDEF and androgen receptor expression vectors were co-transfected together with the PSA promoter luciferase construct and cells were cultured either in the absence or presence of androgen DHT (Dihydro-Testosterone). Conditions for transfections include: Transfections of 3×10⁵ CV-1 cells were carried out with 3 μg reporter gene construct DNA and 1 μg expression vector DNA using 12.5 ml lipofectamine (Gibco-BRL) as described. The cells were either harvested 16 hours after transfection or starved in androgen-free medium containing 10% charcoal stripped FCS for 24 h and then induced with androgen for 24 h and assayed for luciferase activity. Transfections for every construct were performed independently in duplicate and repeated 3 to 4 times with two different plasmid preparations with similar results. Cotransfection of a second plasmid for determination of transfection efficiency was omitted because potential artifacts with this technique have been reported and because many commonly used viral promoters contain potential binding sites for Ets factors. The PSA/luciferase construct (pGL2/PSA-628) was constructed as a fusion protein using the luciferase pGL2 vector (Promega) (Sun, Z, Pan, J, and Balk, S. P. Nucl. Acids Res. 15: 3318-3325 (1997)). (See FIG. 11.)

PDEF in the absence of androgen enhanced PSA promoter activity ˜4-fold. Similarly the androgen receptor in the presence of androgen enhanced the PSA promoter ˜5-fold. In the presence of PDEF, androgen receptor and androgen a synergistic effect of PSA promoter transactivation was observed indicating that PDEF and androgen receptor cooperate in the regulation of the PSA promoter.

### Example 32

### Differential Expression of PDEF in Human Tumors

In order to evaluate expression levels of PDEF in human cancer cell lines and primary cancer tissues, mRNA from cancer cell lines and primary cancer tissues were isolated. RT/PCR analysis revealed a highly differential expression pattern for PDEF in different cancers of epithelial origin. To confirm that similar amounts of cDNA were used for each reaction, RT/PCR with GAPDH specific primers was performed according to the methods described in Example 3 of present invention. Three out of seven lung cancer cell lines express PDEF, but among cervical carcinoma cell lines only A-431, but not HeLa or C-33A cells express PDEF.

Among primary tumors, similar results of differential expression were observed. Whereas one out of three colon cancers, two breast cancers, two prostate cancers, an endometrial cancer, and two out of three Wilms tumors expressed PDEF transcripts, a liver cancer, a pancreatic cancer and an ovarian cancer showed no evidence of PDEF expression. These results suggest that a variety of tumors express high levels of PDEF, but in a certain portion of these or other tumors PDEF is not expressed.

Types of primary tumors and cell lines used to evaluate PDEF expression include: SCC-4 (squamous carcinoma of the tongue), SCC-9 (squamous carcinoma of the tongue), SCC-13 (epidermal squamous carcinome), SCC-15 (squamous carcinoma of the tongue), SCC-40 (soft palate squamous carcinoma), HaCaT (spontaneously immortalized skin keratinocyte, p53 mutant), HeLa (cervical carcinoma), A431 (vulvar carcinoma), C-33A (cervical carcinoma), SK-MES-1 (squamous lung carcinoma), ChaGo K-1 (undifferentiated bronchogenic carcinoma), Calu 1 (epidermoid lung carcinoma, grade E), H157 (large cell lung carcinoma), Calu 6 (lung adenocarcinoma), A549 (lung adenocarcinoma), H249 (small cell lung carcinoma), IGR3 (melanoma), U-373 Mg (astrocytoma, grade III), 340 (glioblastoma), Cl 229 (glioblastoma), A 172 (glioblastoma), T47 (glioma), U-706T (giant cell glioblastoma), U-343 Mg (glioblastoma), U-Cl 2:6 (glioblastoma), U-1242 Mg (glioblastoma), and U-563 Mg (glioblastoma). (See, e.g., Libermann, T. A. and D. Baltimore, “A Pre-B-Cell-Specific Enhancer Element in the Immunoglobulin Heavy-Chain Enhancer,” Mol. Cell. Biol. 13(10): 5957-69 (1993); Libermann, T. A., Friesel, R., Jaye, M., Lyall, R. M., Westermark, B., Drohan, W., Schmidt, A., Maciag, T., and Schlessinger, J., “An Angiogenic Growth Factor is Expressed in Human Glioma Cells,” Embo J., 6: 1627-1632 (1987); Oettgen, P., Akbarali, Y., Boltax, J., Best, J., Kunsch, C. and Libermann, T. A., “Characterization of NERF, a Novel Transcription Factor Related to the Ets Factor ELF-1,” Mol. Cell. Biol., 16: 5091-5106 (1996); Rheinwald, J. G., and M. A. Beckett, “Tumorigenic Keratinocyte Lines Requiring Anchorage and Fibroblast Support Cultures from Human Squamous Cell Carcinomas,” Cancer Res., 41(5): 1657-63 (1981).

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Moreover, the sequence listing is herein incorporated by reference.

TABLE 1

| | | | | | | | | | Kyle- . . . | Eisen . . . | Eisen . . . | Karpl . . . | James . . . | Emini |
|---|---|---|---|---|---|---|---|---|---|---|---|---|---|---|
| Res | Pos | Gami . . . Alpha | Chou- . . . Alpha | Gami . . . Beta | Chou- . . . Beta | Gami . . . Turn | Chou- . . . Turn | Gami . . . Coil | Hydro . . . | Alpha | Beta | Flexi . . . | Antig . . . | Surfa . . . |
| Met | 1 | . | . | B | . | . | . | . | −0.19 | . | . | . | −0.10 | 0.50 |
| Gly | 2 | . | . | B | . | . | . | . | −0.01 | . | . | . | −0.10 | 0.52 |
| Ser | 3 | . | . | B | . | . | . | . | 0.03 | . | . | . | −0.10 | 0.63 |
| Ala | 4 | . | . | . | . | . | . | C | −0.39 | . | . | . | 0.10 | 0.63 |
| Ser | 5 | . | . | . | . | . | T | C | −0.30 | . | . | F | 0.45 | 0.52 |
| Pro | 6 | . | . | . | . | . | T | C | −0.00 | . | . | F | 0.45 | 0.52 |
| Gly | 7 | . | . | . | . | T | T | . | −0.51 | . | . | F | 0.65 | 0.70 |
| Leu | 8 | . | . | B | . | . | T | . | −0.51 | . | . | F | 0.25 | 0.39 |
| Ser | 9 | . | . | B | B | . | . | . | −0.13 | . | . | F | −0.15 | 0.33 |
| Ser | 10 | . | . | B | B | . | . | . | −0.13 | . | . | F | −0.15 | 0.52 |
| Val | 11 | . | . | B | B | . | . | . | 0.04 | . | . | F | −0.15 | 0.85 |
| Ser | 12 | . | . | B | . | . | T | . | −0.42 | . | . | F | 0.25 | 0.86 |
| Pro | 13 | . | . | B | . | . | T | . | −0.42 | . | . | F | −0.05 | 0.53 |
| Ser | 14 | . | . | B | . | . | T | . | −0.93 | . | . | F | −0.05 | 0.59 |
| His | 15 | . | . | B | . | . | T | . | −0.84 | . | . | . | −0.20 | 0.36 |
| Leu | 16 | . | . | B | . | . | . | . | −0.20 | . | . | . | −0.40 | 0.36 |
| Leu | 17 | . | . | B | . | . | . | . | 0.10 | . | . | . | −0.40 | 0.42 |
| Leu | 18 | . | . | B | . | . | . | . | 0.00 | . | . | . | −0.40 | 0.51 |
| Pro | 19 | . | . | B | . | . | T | . | −0.56 | . | . | F | −0.05 | 0.90 |
| Pro | 20 | . | . | . | . | T | T | . | −0.82 | * | * | F | 0.65 | 0.81 |
| Asp | 21 | . | . | . | . | T | T | . | 0.10 | * | . | F | 0.80 | 1.31 |
| Thr | 22 | . | . | B | . | . | T | . | 0.60 | * | | F | 1.30 | 1.66 |
| Val | 23 | . | . | B | . | . | . | . | 1.07 | * | * | F | 1.10 | 1.55 |
| Ser | 24 | . | . | B | . | . | T | . | 0.47 | * | * | F | 1.15 | 0.92 |
| Arg | 25 | . | . | B | . | . | T | . | 0.68 | * | . | F | 0.25 | 0.53 |
| Thr | 26 | . | . | B | . | . | T | . | 0.72 | * | . | F | 1.00 | 1.23 |
| Gly | 27 | A | . | . | . | . | T | . | 0.44 | * | . | F | 1.30 | 1.83 |
| Leu | 28 | A | A | . | . | . | . | . | 0.71 | * | . | F | 0.75 | 0.94 |
| Glu | 29 | A | A | . | . | . | . | . | 0.42 | * | . | F | 0.45 | 0.66 |
| Lys | 30 | A | A | . | . | . | . | . | −0.03 | * | * | . | 0.30 | 0.68 |
| Ala | 31 | A | A | . | . | . | . | . | −0.31 | * | . | . | 0.30 | 0.81 |
| Ala | 32 | A | A | . | . | . | . | . | −0.82 | . | . | . | 0.60 | 0.47 |
| Ala | 33 | A | A | . | . | . | . | . | −0.36 | * | . | . | −0.30 | 0.18 |
| Gly | 34 | A | A | . | . | . | . | . | −1.17 | . | . | . | −0.60 | 0.17 |
| Ala | 35 | A | A | . | . | . | . | . | −1.21 | * | . | . | −0.60 | 0.14 |
| Val | 36 | A | A | . | . | . | . | . | −0.51 | * | . | . | −0.30 | 0.24 |
| Gly | 37 | A | A | . | . | . | . | . | 0.19 | . | . | . | 0.64 | 0.48 |
| Leu | 38 | . | A | B | . | . | . | . | 0.78 | . | . | . | 1.28 | 0.92 |
| Glu | 39 | . | A | B | . | . | . | . | 0.83 | . | . | F | 1.92 | 2.08 |
| Arg | 40 | . | . | B | . | . | T | . | 1.12 | . | . | F | 2.66 | 2.21 |
| Arg | 41 | . | . | . | . | T | T | . | 1.77 | . | . | F | 3.40 | 3.58 |
| Asp | 42 | . | . | . | . | T | T | . | 1.81 | . | . | F | 3.06 | 3.20 |
| Trp | 43 | . | . | . | . | T | T | . | 2.41 | . | . | F | 2.72 | 2.19 |
| Ser | 44 | . | . | . | . | . | T | C | 2.20 | . | . | F | 1.88 | 1.73 |
| Pro | 45 | . | . | . | . | . | T | C | 1.50 | . | . | F | 1.54 | 1.60 |
| Ser | 46 | . | . | . | . | . | T | C | 1.08 | . | . | F | 0.50 | 1.54 |
| Pro | 47 | . | . | . | . | . | T | C | 0.87 | . | * | F | 1.00 | 1.66 |
| Pro | 48 | . | . | . | . | . | . | C | 1.16 | . | . | F | 1.00 | 1.66 |
| Ala | 49 | . | . | . | . | . | . | C | 1.46 | . | . | F | 1.80 | 2.14 |
| Thr | 50 | . | . | B | . | . | T | . | 1.32 | . | . | F | 2.00 | 2.40 |
| Pro | 51 | . | . | B | . | . | T | . | 0.81 | . | . | F | 1.80 | 1.53 |
| Glu | 52 | . | . | B | . | . | T | . | 0.72 | . | . | F | 1.60 | 1.25 |
| Gln | 53 | . | . | B | . | . | T | . | 0.34 | . | . | F | 1.40 | 1.16 |
| Gly | 54 | . | . | B | . | . | . | . | 0.23 | . | . | F | 0.85 | 0.76 |
| Leu | 55 | . | . | B | B | . | . | . | 0.30 | . | . | . | −0.30 | 0.38 |
| Ser | 56 | . | . | B | B | . | . | . | −0.30 | . | . | . | −0.60 | 0.34 |
| Ala | 57 | . | . | B | B | . | . | . | −0.60 | . | . | . | −0.60 | 0.29 |
| Phe | 58 | . | . | B | B | . | . | . | −0.84 | . | . | . | −0.60 | 0.47 |
| Tyr | 59 | . | . | B | B | . | . | . | −1.20 | . | . | . | −0.60 | 0.54 |
| Leu | 60 | . | . | B | B | . | . | . | −0.39 | . | . | . | −0.60 | 0.47 |
| Ser | 61 | . | . | B | B | . | . | . | −0.69 | . | . | . | −0.60 | 0.90 |
| Tyr | 62 | . | . | B | B | . | . | . | −0.91 | . | . | . | −0.60 | 0.57 |
| Phe | 63 | . | . | B | B | . | . | . | −0.46 | . | . | . | −0.60 | 0.57 |
| Asp | 64 | . | . | B | B | . | . | . | −0.42 | . | . | . | −0.60 | 0.66 |
| Met | 65 | . | . | B | B | . | . | . | 0.39 | . | . | . | −0.60 | 0.66 |
| Leu | 66 | . | . | B | B | . | . | . | 0.69 | . | . | . | −0.15 | 1.31 |
| Tyr | 67 | . | . | B | B | . | . | . | 0.63 | . | . | . | 0.75 | 1.31 |
| Pro | 68 | . | . | . | B | . | . | C | 1.03 | . | . | F | 0.80 | 1.78 |
| Glu | 69 | A | . | . | . | . | . | . | 0.74 | . | . | F | 0.80 | 2.88 |
| Asp | 70 | A | . | . | . | . | T | . | 0.76 | . | . | F | 1.00 | 1.94 |
| Ser | 71 | A | . | . | . | . | T | . | 0.98 | . | * | F | 1.00 | 1.26 |
| Ser | 72 | A | . | . | . | . | T | . | 1.27 | . | * | F | 0.85 | 0.74 |
| Trp | 73 | A | . | . | . | . | T | . | 0.89 | . | * | . | 0.91 | 0.88 |
| Ala | 74 | A | . | . | . | . | . | . | 0.68 | . | * | . | 0.32 | 0.67 |
| Ala | 75 | A | . | . | . | . | . | . | 0.33 | . | * | . | 0.53 | 0.77 |
| Lys | 76 | A | . | . | . | . | . | . | 0.04 | . | * | F | 0.89 | 0.72 |
| Ala | 77 | . | . | . | . | . | T | C | 0.04 | . | * | F | 2.10 | 0.72 |
| Pro | 78 | . | . | . | . | . | T | C | 0.03 | * | * | F | 1.89 | 0.96 |
| Gly | 79 | . | . | . | . | . | T | C | 0.73 | * | . | F | 1.68 | 0.64 |
| Ala | 80 | . | . | . | . | . | T | C | 1.32 | . | * | F | 1.62 | 1.25 |
| Ser | 81 | . | . | . | . | . | T | C | 1.28 | . | * | F | 1.71 | 1.40 |
| Ser | 82 | . | . | . | . | . | T | C | 1.66 | . | * | F | 1.50 | 2.44 |
| Arg | 83 | . | . | . | . | . | T | C | 1.66 | . | . | F | 1.84 | 3.74 |
| Glu | 84 | . | . | . | . | . | T | C | 2.00 | . | * | F | 2.18 | 4.31 |
| Glu | 85 | . | . | . | . | . | . | C | 2.59 | . | * | F | 2.32 | 5.58 |
| Pro | 86 | . | . | . | . | . | T | C | 2.68 | . | * | F | 2.86 | 4.93 |
| Pro | 87 | . | . | . | . | T | T | . | 2.98 | . | . | F | 3.40 | 4.40 |
| Glu | 88 | . | . | . | . | T | T | . | 2.87 | . | * | F | 3.06 | 4.40 |
| Glu | 89 | A | . | . | . | . | T | . | 2.20 | * | . | F | 2.32 | 4.93 |
| Pro | 90 | A | . | . | . | . | . | . | 1.99 | . | . | F | 1.78 | 1.71 |
| Glu | 91 | A | . | . | . | . | . | . | 1.34 | . | . | F | 1.44 | 1.53 |
| Gln | 92 | A | . | . | B | . | . | . | 0.67 | . | . | F | 0.75 | 0.65 |
| Cys | 93 | . | . | B | B | . | . | . | 0.67 | . | . | . | −0.30 | 0.30 |
| Pro | 94 | . | . | B | B | . | . | . | 0.37 | . | . | . | 0.30 | 0.29 |
| Val | 95 | . | . | B | B | . | . | . | 0.58 | . | . | . | −0.30 | 0.22 |
| Ile | 96 | . | . | B | B | . | . | . | −0.01 | . | . | . | −0.30 | 0.71 |
| Asp | 97 | . | . | B | B | . | . | . | −0.22 | . | * | F | −0.15 | 0.47 |
| Ser | 98 | . | . | B | . | . | . | . | −0.14 | . | * | F | 0.05 | 0.97 |
| Gln | 99 | . | . | B | . | . | . | . | −0.28 | . | * | F | 0.80 | 1.40 |
| Ala | 100 | . | . | . | . | . | . | C | 0.28 | . | . | F | 0.85 | 0.83 |
| Pro | 101 | . | . | . | . | . | T | C | 0.36 | . | * | F | 0.45 | 0.83 |
| Ala | 102 | . | . | . | . | T | T | . | 0.36 | . | * | F | 0.65 | 0.40 |
| Gly | 103 | . | . | B | . | . | T | . | −0.16 | . | . | F | 0.85 | 0.65 |
| Ser | 104 | . | . | B | . | . | T | . | −1.01 | . | . | F | 0.25 | 0.35 |
| Leu | 105 | . | . | B | . | . | . | . | −0.63 | . | . | . | −0.10 | 0.26 |
| Asp | 106 | . | . | B | . | . | . | . | −0.77 | . | . | . | −0.10 | 0.40 |
| Leu | 107 | . | . | B | . | . | . | . | −0.52 | . | . | . | −0.10 | 0.30 |
| Val | 108 | . | . | B | . | . | T | . | −0.99 | . | * | F | 0.25 | 0.35 |
| Pro | 109 | . | . | B | . | . | T | . | −1.00 | . | * | F | 0.25 | 0.18 |
| Gly | 110 | . | . | . | . | . | T | C | −1.00 | . | * | F | 0.15 | 0.31 |
| Gly | 111 | . | . | . | . | . | T | C | −1.00 | . | . | F | 0.15 | 0.34 |
| Leu | 112 | A | A | . | . | . | . | . | −0.19 | . | * | F | −0.15 | 0.38 |
| Thr | 113 | A | A | . | . | . | . | . | 0.63 | . | * | . | 0.30 | 0.67 |
| Leu | 114 | A | A | . | . | . | . | . | 0.54 | . | . | . | 0.30 | 0.92 |
| Glu | 115 | A | A | . | . | . | . | . | 0.08 | . | . | . | 0.45 | 1.49 |
| Glu | 116 | A | A | . | . | . | . | . | 0.42 | . | . | . | 0.30 | 0.85 |
| His | 117 | A | A | . | . | . | . | . | 1.23 | . | . | . | 0.75 | 1.79 |
| Ser | 118 | A | A | . | . | . | . | . | 0.69 | . | . | . | 0.75 | 1.79 |
| Leu | 119 | A | A | . | . | . | . | . | 1.50 | . | . | . | 0.30 | 0.77 |
| Glu | 120 | A | A | . | . | . | . | . | 1.20 | . | . | . | −0.30 | 0.98 |
| Gln | 121 | A | A | . | . | . | . | . | 0.60 | * | . | F | 0.45 | 0.98 |
| Val | 122 | A | A | . | . | . | . | . | −0.22 | * | . | F | 0.00 | 1.17 |
| Gln | 123 | A | A | . | . | . | . | . | −0.78 | . | . | . | −0.30 | 0.50 |
| Ser | 124 | . | . | B | B | . | . | . | −0.31 | . | . | . | −0.60 | 0.22 |
| Met | 125 | . | . | B | B | . | . | . | −0.31 | . | * | . | −0.60 | 0.29 |
| Val | 126 | . | A | B | B | . | . | . | −1.17 | . | * | . | −0.30 | 0.29 |
| Val | 127 | A | A | . | B | . | . | . | −1.12 | . | * | . | −0.30 | 0.16 |
| Gly | 128 | A | A | . | B | . | . | . | −1.08 | * | . | . | −0.60 | 0.13 |
| Glu | 129 | A | A | . | B | . | . | . | −0.78 | * | . | . | −0.30 | 0.36 |
| Val | 130 | A | A | . | B | . | . | . | −1.07 | * | . | . | 0.60 | 0.80 |
| Leu | 131 | A | A | . | B | . | . | . | −0.21 | * | . | F | 0.75 | 0.57 |
| Lys | 132 | A | A | . | B | . | . | . | 0.33 | * | . | F | 0.75 | 0.57 |
| Asp | 133 | A | A | . | B | . | . | . | 0.09 | * | . | F | 0.60 | 1.11 |
| Ile | 134 | A | A | . | B | . | . | . | −0.58 | * | * | F | 0.90 | 1.36 |
| Glu | 135 | A | A | . | B | . | . | . | 0.32 | * | * | F | 0.75 | 0.36 |
| Thr | 136 | A | A | . | . | . | . | . | 0.32 | * | * | F | 0.75 | 0.43 |
| Ala | 137 | A | A | . | . | . | . | . | −0.53 | * | . | . | 0.30 | 0.51 |
| Cys | 138 | A | A | . | . | . | . | . | −0.53 | * | * | . | 0.30 | 0.24 |
| Lys | 139 | A | A | . | . | . | . | . | −0.53 | * | * | . | −0.30 | 0.27 |
| Leu | 140 | A | A | . | B | . | . | . | −0.84 | * | . | . | −0.60 | 0.19 |
| Leu | 141 | A | A | . | B | . | . | . | −1.12 | * | * | . | −0.60 | 0.51 |
| Asn | 142 | . | A | B | B | . | . | . | −0.53 | * | * | . | −0.60 | 0.26 |
| Ile | 143 | . | A | B | B | . | . | . | −0.08 | * | * | . | −0.60 | 0.52 |
| Thr | 144 | . | A | B | B | . | . | . | −0.72 | * | . | . | −0.05 | 0.97 |
| Ala | 145 | . | A | B | B | . | . | . | 0.09 | * | * | F | 0.35 | 0.60 |
| Asp | 146 | . | . | B | B | . | . | . | 0.61 | . | * | F | 1.35 | 1.43 |
| Pro | 147 | . | . | B | . | . | T | . | 0.31 | . | * | F | 2.00 | 1.04 |
| Met | 148 | . | . | . | . | T | T | . | 0.99 | . | * | . | 2.50 | 1.38 |
| Asp | 149 | . | . | . | . | T | T | . | 1.00 | . | . | . | 2.25 | 1.28 |
| Trp | 150 | . | . | . | . | . | T | C | 1.59 | . | . | . | 1.20 | 1.11 |
| Ser | 151 | . | . | . | . | . | T | C | 0.73 | . | . | F | 1.10 | 1.80 |
| Pro | 152 | . | . | . | . | . | T | C | 0.94 | * | . | F | 0.70 | 0.80 |
| Ser | 153 | . | . | . | . | T | T | . | 1.59 | * | . | F | 0.50 | 1.32 |
| Asn | 154 | . | . | . | . | . | T | C | 1.30 | * | . | F | 1.20 | 1.96 |
| Val | 155 | . | A | . | . | . | . | C | 0.78 | . | . | F | 0.20 | 1.34 |
| Gin | 156 | . | A | . | . | T | . | . | 0.27 | . | . | F | −0.05 | 0.82 |
| Lys | 157 | . | A | B | . | . | . | . | 0.19 | . | . | . | −0.60 | 0.42 |
| Trp | 158 | . | A | B | . | . | . | . | 0.18 | . | . | . | −0.60 | 0.60 |
| Leu | 159 | . | A | B | . | . | . | . | 0.18 | . | . | . | −0.60 | 0.50 |
| Leu | 160 | . | A | B | . | . | . | . | 1.00 | . | . | . | −0.60 | 0.43 |
| Trp | 161 | A | A | . | . | . | . | . | 1.00 | . | . | . | −0.60 | 0.56 |
| Thr | 162 | A | A | . | . | . | . | . | 0.71 | * | * | . | −0.45 | 1.17 |
| Glu | 163 | . | A | . | . | T | . | . | 1.11 | . | * | . | −0.05 | 2.22 |
| His | 164 | . | A | . | . | T | . | . | 1.11 | . | * | . | 0.25 | 4.14 |
| Gln | 165 | . | A | . | . | T | . | . | 1.71 | . | * | . | 0.85 | 2.37 |
| Tyr | 166 | . | . | . | . | T | . | . | 1.79 | * | * | . | 1.30 | 2.11 |
| Arg | 167 | . | . | . | . | T | . | . | 1.50 | . | * | . | 0.95 | 2.40 |
| Leu | 168 | . | . | . | . | . | . | C | 1.16 | * | * | . | 1.00 | 1.37 |
| Pro | 169 | . | . | . | . | . | . | C | 1.23 | . | * | F | 1.25 | 0.87 |
| Pro | 170 | . | . | . | . | T | T | . | 0.64 | * | * | F | 2.50 | 0.89 |
| Met | 171 | . | . | . | . | T | T | . | 0.19 | * | * | F | 1.80 | 1.08 |
| Gly | 172 | . | . | . | . | . | T | C | 0.08 | * | * | F | 1.20 | 0.61 |
| Lys | 173 | A | . | . | . | . | T | . | 0.89 | * | . | F | 0.75 | 0.68 |
| Ala | 174 | A | A | . | . | . | . | . | 0.29 | * | . | . | 0.70 | 1.19 |
| Phe | 175 | A | A | . | . | . | . | . | −0.09 | * | . | . | 0.30 | 0.99 |
| Gln | 176 | A | A | . | . | . | . | . | 0.17 | * | * | . | 0.30 | 0.50 |
| Glu | 177 | A | A | . | . | . | . | . | 0.56 | * | . | . | −0.30 | 0.49 |
| Leu | 178 | A | A | . | . | . | . | . | 0.51 | * | . | . | 0.45 | 1.13 |
| Ala | 179 | A | A | . | . | . | . | . | 0.29 | . | . | F | 0.90 | 1.13 |
| Gly | 180 | A | A | . | . | . | . | . | 0.32 | . | . | F | 0.75 | 0.54 |
| Lys | 181 | A | A | . | . | . | . | . | −0.27 | . | . | F | 0.45 | 0.35 |
| Glu | 182 | A | A | . | . | . | . | . | −0.87 | . | . | F | 0.45 | 0.35 |
| Leu | 183 | A | A | . | . | . | . | . | −0.36 | . | . | . | 0.30 | 0.35 |
| Cys | 184 | A | A | . | . | . | . | . | 0.23 | * | . | . | 0.30 | 0.24 |
| Ala | 185 | A | A | . | . | . | . | . | 0.58 | . | . | . | 0.30 | 0.24 |
| Mel | 186 | A | A | . | . | . | . | . | 0.53 | . | . | . | 0.30 | 0.49 |
| Ser | 187 | A | A | . | . | . | . | . | −0.17 | * | * | . | 0.75 | 1.60 |
| Glu | 188 | A | A | . | . | . | . | . | 0.76 | * | * | F | 0.60 | 1.37 |
| Glu | 189 | A | A | . | . | . | . | . | 1.42 | . | * | F | 0.90 | 2.71 |
| Gln | 190 | A | A | . | . | . | . | . | 2.12 | . | * | F | 0.90 | 3.50 |
| Phe | 191 | A | A | . | . | . | . | . | 2.42 | . | * | F | 0.90 | 3.96 |
| Arg | 192 | A | A | . | . | . | . | . | 2.51 | . | * | F | 0.90 | 3.06 |
| Gln | 193 | . | A | . | . | T | . | . | 1.70 | . | * | F | 1.61 | 2.73 |
| Arg | 194 | . | A | . | . | T | . | . | 1.36 | . | * | F | 1.62 | 2.60 |
| Ser | 195 | . | . | . | . | . | T | C | 1.01 | . | * | F | 2.43 | 1.32 |
| Pro | 196 | . | . | . | . | . | T | C | 1.71 | . | * | F | 2.29 | 0.75 |
| Leu | 197 | . | . | . | . | T | T | . | 0.74 | . | * | F | 3.10 | 0.64 |
| Gly | 198 | . | . | . | . | T | T | . | −0.07 | . | * | F | 1.89 | 0.35 |
| Gly | 199 | . | . | . | . | . | . | C | −0.21 | . | * | F | 1.18 | 0.19 |
| Asp | 200 | . | A | B | . | . | . | . | −0.50 | . | . | F | 0.47 | 0.31 |
| Val | 201 | . | A | B | . | . | . | . | −0.32 | . | * | . | 0.01 | 0.32 |
| Leu | 202 | A | A | . | . | . | . | . | −0.32 | * | * | . | −0.30 | 0.44 |
| His | 203 | A | A | . | . | . | . | . | 0.02 | * | * | . | −0.60 | 0.22 |
| Ala | 204 | A | A | . | . | . | . | . | −0.52 | * | * | . | −0.60 | 0.49 |
| His | 205 | A | A | . | . | . | . | . | −0.81 | * | * | . | −0.60 | 0.41 |
| Leu | 206 | A | A | . | . | . | . | . | 0.09 | * | * | . | −0.60 | 0.32 |
| Asp | 207 | A | A | . | . | . | . | . | 0.60 | * | * | . | −0.30 | 0.63 |
| Ile | 208 | A | A | . | . | . | . | . | 0.04 | * | * | . | −0.30 | 0.62 |
| Trp | 209 | A | A | . | . | . | . | . | 0.04 | * | * | . | −0.30 | 0.76 |
| Lys | 210 | A | A | . | . | . | . | . | −0.21 | . | . | . | 0.30 | 0.46 |
| Ser | 211 | A | A | . | . | . | . | . | −0.00 | . | * | . | −0.60 | 0.69 |
| Ala | 212 | A | A | . | . | . | . | . | 0.04 | * | . | . | −0.60 | 0.65 |
| Ala | 213 | A | A | . | . | . | . | . | 0.93 | * | * | . | 0.30 | 0.65 |
| Trp | 214 | A | A | . | . | . | . | . | 1.33 | . | * | . | 0.30 | 0.84 |
| Met | 215 | A | A | . | . | . | . | . | 0.98 | . | * | . | 0.45 | 1.63 |
| Lys | 216 | A | A | . | . | . | . | . | 0.98 | . | * | . | 0.75 | 2.34 |
| Glu | 217 | A | A | . | . | . | . | . | 1.36 | . | * | F | 1.50 | 2.98 |
| Arg | 218 | . | A | . | . | T | . | . | 1.60 | . | * | F | 2.20 | 4.65 |
| Thr | 219 | . | A | . | . | . | . | C | 1.30 | . | * | F | 2.30 | 2.30 |
| Ser | 220 | . | . | . | . | . | T | C | 1.01 | . | * | F | 3.00 | 1.34 |
| Pro | 221 | . | . | . | . | T | T | . | 0.93 | . | * | F | 2.45 | 0.48 |
| Gly | 222 | . | . | . | . | T | T | . | 0.69 | * | . | F | 1.25 | 0.45 |
| Ala | 223 | . | . | B | . | . | T | . | −0.09 | * | * | . | 0.40 | 0.53 |
| Ile | 224 | . | . | B | . | . | . | . | −0.37 | . | . | . | −0.10 | 0.18 |
| His | 225 | . | . | B | . | . | . | . | −0.37 | . | . | . | −0.40 | 0.19 |
| Tyr | 226 | . | . | B | . | . | . | . | −0.47 | . | . | . | −0.40 | 0.25 |
| Cys | 227 | . | . | B | . | . | T | . | −0.42 | . | . | . | −0.20 | 0.51 |
| Ala | 228 | . | . | B | . | . | T | . | 0.17 | . | . | . | −0.20 | 0.50 |
| Ser | 229 | . | . | . | . | . | T | C | 1.06 | . | . | F | 0.45 | 0.56 |
| Thr | 230 | . | . | . | . | . | T | C | 0.79 | . | . | F | 1.80 | 1.80 |
| Ser | 231 | . | . | . | . | . | . | C | 0.74 | . | . | F | 1.90 | 2.39 |
| Glu | 232 | A | . | . | . | . | . | . | 1.10 | . | . | F | 1.70 | 1.87 |
| Glu | 233 | A | . | . | . | . | . | . | 1.69 | . | . | F | 2.00 | 1.87 |
| Ser | 234 | . | . | . | . | T | . | . | 1.69 | . | . | F | 3.00 | 2.33 |
| Trp | 235 | A | . | . | . | . | T | . | 2.00 | . | . | F | 2.50 | 1.81 |
| Thr | 236 | A | . | . | . | . | T | . | 1.44 | . | * | F | 2.54 | 1.81 |
| Asp | 237 | A | . | . | . | . | T | . | 1.44 | . | * | F | 2.28 | 1.00 |
| Ser | 238 | A | . | . | . | . | T | . | 1.14 | . | * | F | 2.62 | 1.59 |
| Glu | 239 | A | . | . | . | T | . | . | 1.14 | . | * | F | 2.86 | 1.48 |
| Val | 240 | . | . | . | . | T | T | . | 0.77 | . | * | F | 3.40 | 1.18 |
| Asp | 241 | . | . | . | . | T | T | . | 0.78 | . | * | F | 2.91 | 0.47 |
| Ser | 242 | . | . | . | . | T | T | . | 0.43 | . | * | F | 2.70 | 0.37 |
| Ser | 243 | . | . | . | . | T | T | . | 0.73 | . | * | F | 2.19 | 0.49 |
| Cys | 244 | . | . | . | . | T | T | . | 0.52 | . | * | F | 1.98 | 0.51 |
| Ser | 245 | . | . | . | . | T | T | . | 0.49 | . | * | F | 1.77 | 0.58 |
| Gly | 246 | . | . | . | . | T | T | . | 0.46 | . | * | F | 1.30 | 0.31 |
| Gln | 247 | . | . | . | . | . | T | C | −0.06 | . | . | F | 0.97 | 0.78 |
| Pro | 248 | . | . | B | . | . | . | . | −0.04 | . | . | F | 0.14 | 0.48 |
| Ile | 249 | . | A | B | . | . | . | . | 0.62 | . | . | . | −0.34 | 0.51 |
| His | 250 | . | A | B | . | . | . | . | 0.22 | . | . | . | −0.47 | 0.51 |
| Leu | 251 | . | A | B | . | . | . | . | −0.24 | . | . | . | −0.60 | 0.28 |
| Trp | 252 | A | A | . | . | . | . | . | −0.20 | * | . | . | −0.60 | 0.33 |
| Gln | 253 | A | A | . | . | . | . | . | 0.01 | * | . | . | −0.60 | 0.49 |
| Phe | 254 | A | A | . | . | . | . | . | 0.09 | * | . | . | −0.45 | 1.03 |
| Leu | 255 | A | A | . | . | . | . | . | −0.69 | * | . | . | −0.60 | 0.81 |
| Lys | 256 | A | A | . | . | . | . | . | −0.69 | * | . | . | −0.30 | 0.39 |
| Glu | 257 | A | A | . | . | . | . | . | −0.36 | * | * | . | −0.60 | 0.37 |
| Leu | 258 | A | A | . | . | . | . | . | −0.57 | * | * | . | 0.30 | 0.89 |
| Leu | 259 | A | A | . | . | . | . | . | 0.10 | * | . | . | 0.60 | 0.69 |
| Leu | 260 | A | A | . | . | . | . | . | 0.61 | * | . | . | 0.30 | 0.54 |
| Lys | 261 | A | A | . | . | . | . | . | 0.32 | . | * | F | −0.15 | 0.88 |
| Pro | 262 | A | A | . | . | . | . | . | −0.02 | * | * | F | −0.30 | 1.67 |
| His | 263 | . | . | . | . | T | T | . | 0.90 | . | * | F | 0.80 | 2.00 |
| Ser | 264 | . | . | . | . | T | T | . | 1.01 | * | * | . | 1.25 | 1.98 |
| Tyr | 265 | . | . | B | . | . | T | . | 0.93 | * | * | . | 0.25 | 1.10 |
| Gly | 266 | . | . | B | . | . | T | . | 1.00 | * | * | . | −0.20 | 0.57 |
| Arg | 267 | . | A | B | . | . | . | . | 0.92 | * | . | . | −0.30 | 0.83 |
| Phe | 268 | . | A | B | . | . | . | . | 0.14 | * | . | . | −0.60 | 0.56 |
| Ile | 269 | . | A | B | . | . | . | . | 0.44 | * | . | . | −0.60 | 0.46 |
| Arg | 270 | . | A | B | . | . | . | . | 0.73 | * | . | . | −0.60 | 0.38 |
| Trp | 271 | A | A | . | . | . | . | . | 1.08 | * | * | . | −0.60 | 0.88 |
| Leu | 272 | A | A | . | . | . | . | . | 1.01 | * | * | . | 0.45 | 2.17 |
| Asn | 273 | A | A | . | . | . | . | . | 1.37 | * | * | F | 0.90 | 2.21 |
| Lys | 274 | A | A | . | . | . | . | . | 1.37 | * | * | F | 0.90 | 2.08 |
| Glu | 275 | . | A | . | . | T | . | . | 0.56 | * | * | F | 1.30 | 1.77 |
| Lys | 276 | A | A | . | . | . | . | . | 0.89 | * | * | F | 0.75 | 0.95 |
| Gly | 277 | A | A | . | . | . | . | . | 0.81 | . | * | F | 0.75 | 0.95 |
| Ile | 278 | A | A | . | . | . | . | . | 0.81 | * | * | . | 0.30 | 0.39 |
| Phe | 279 | . | A | B | . | . | . | . | 0.77 | * | * | . | 0.30 | 0.33 |
| Lys | 280 | A | A | . | . | . | . | . | 0.47 | * | * | . | 0.30 | 0.56 |
| Ile | 281 | A | A | . | . | . | . | . | −0.17 | * | * | F | 0.60 | 1.08 |
| Glu | 282 | A | A | . | . | . | . | . | 0.18 | * | * | F | 0.90 | 1.26 |
| Asp | 283 | A | A | . | . | . | . | . | 0.21 | * | * | F | 0.90 | 1.09 |
| Ser | 284 | A | A | . | . | . | . | . | 0.32 | . | * | F | 0.60 | 1.15 |
| Ala | 285 | A | A | . | . | . | . | . | 0.39 | * | * | . | 0.60 | 0.67 |
| Gln | 286 | A | A | . | . | . | . | . | 0.47 | * | * | . | 0.60 | 0.79 |
| Val | 287 | A | A | . | . | . | . | . | 0.18 | * | . | . | −0.30 | 0.49 |
| Ala | 288 | A | A | . | . | . | . | . | −0.17 | . | . | . | −0.60 | 0.51 |
| Arg | 289 | A | A | . | . | . | . | . | −0.76 | . | . | . | −0.60 | 0.29 |
| Leu | 290 | . | A | B | . | . | . | . | −0.06 | . | . | . | −0.60 | 0.27 |
| Trp | 291 | . | A | B | . | . | . | . | −0.01 | . | . | . | 0.04 | 0.53 |
| Gly | 292 | . | A | B | . | . | . | . | 0.84 | . | . | . | 0.98 | 0.54 |
| Ile | 293 | . | . | . | . | T | . | . | 1.54 | . | . | . | 1.47 | 1.05 |
| Arg | 294 | . | . | B | . | . | T | . | 1.22 | . | . | F | 2.66 | 1.96 |
| Lys | 295 | . | . | . | . | T | T | . | 1.44 | . | . | F | 3.40 | 3.07 |
| Asn | 296 | . | . | . | . | . | T | C | 1.13 | * | * | F | 2.86 | 4.42 |
| Arg | 297 | . | . | . | . | . | T | C | 1.48 | * | * | F | 2.52 | 2.23 |
| Pro | 298 | . | . | . | . | . | . | C | 2.12 | * | * | F | 1.98 | 1.79 |
| Ala | 299 | . | . | . | . | T | . | . | 2.01 | * | . | . | 0.79 | 1.75 |
| Met | 300 | . | . | B | . | . | . | . | 2.01 | * | * | . | 0.65 | 1.49 |
| Asn | 301 | . | . | B | . | . | T | . | 1.20 | * | * | . | 0.85 | 1.93 |
| Tyr | 302 | . | . | B | . | . | T | . | 0.79 | * | * | . | 0.85 | 1.57 |
| Asp | 303 | A | . | . | . | . | T | . | 1.11 | * | . | F | 1.00 | 2.13 |
| Lys | 304 | A | . | . | . | . | T | . | 1.40 | * | . | F | 1.30 | 2.60 |
| Leu | 305 | . | . | B | . | . | T | . | 1.11 | * | * | F | 1.30 | 2.22 |
| Ser | 306 | . | . | B | . | . | T | . | 1.22 | * | . | F | 1.15 | 0.93 |
| Arg | 307 | . | . | B | . | . | T | . | 1.47 | * | . | F | 1.15 | 0.91 |
| Ser | 308 | . | . | B | . | . | T | . | 1.22 | * | . | F | 1.00 | 1.92 |

What is claimed is:

1. An isolated polynucleotide comprising a first nucleic acid at least 95% identical to a reference nucleic acid selected from the group consisting of:
(a) a nucleic acid consisting of nucleotides 839 to 1048 of SEQ ID NO:1;
(b) a nucleic acid consisting of nucleotides 419 to 1420 of SEQ ID NO:1;
(c) a nucleic acid consisting of nucleotides 416 to 1420 of SEQ ID NO:1; and
(d) a nucleic acid consisting of the nucleotides encoding the complete amino acid sequence encoded by the cDNA clone contained in ATCC Deposit No. 203072; wherein said first nucleic acid encodes a polypeptide that regulates Prostate-Specific Antigen (PSA) gene expression.

2. The isolated polynucleotide of claim 1, wherein said first nucleic acid is at least 95% identical to a reference nucleic acid consisting of nucleotides 839 to 1048 of SEQ ID NO:1.

3. The isolated polynucleotide of claim 2, comprising nucleotides 839 to 1048 of SEQ ID NO:1.

4. The isolated polynucleotide of claim 1, wherein said first nucleic acid is at least 95% identical to a reference nucleic acid consisting of nucleotides 419 to 1420 of SEQ ID NO:1.

5. The isolated polynucleotide of claim 4, comprising nucleotides 419 to 1420 of SEQ ID NO:1.

6. The isolated polynucleotide of claim 1, wherein said first nucleic acid is at least 95% identical to a reference nucleic acid consisting of nucleotides 416 to 1420 of SEQ ID NO:1.

7. The isolated polynucleotide of claim 1, comprising nucleotides 416 to 1420 of SEQ ID NO:1.

8. The isolated polynucleotide of claim 1, wherein said first nucleic acid is at least 95% identical to a reference nucleic acid consisting of the nucleotides encoding the complete amino acid sequence encoded by the cDNA clone contained in ATCC Deposit No. 203072.

9. The isolated polynucleotide of claim 8, comprising the nucleotides encoding the complete amino acid sequence encoded by the cDNA clone contained in ATCC Deposit No. 203072.

10. The isolated polynucleotide of claim 1, further comprising a nucleotide sequence heterologous to said first nucleic acid.

11. A method of producing a vector comprising inserting the isolated polynucleotide of claim 1 into a vector.

12. A vector comprising the isolated polynucleotide of claim 1.

13. The vector of claim 12, wherein said polynucleotide comprises a nucleotide sequence heterologous to said first nucleic acid.

14. A host cell comprising the isolated polynucleotide of claim 1.

15. The host cell of claim 14, wherein said polynucleotide comprises a nucleotide sequence heterologous to said first nucleic acid.

16. An isolated polynucleotide comprising a nucleic acid encoding a first amino acid sequence at least 99% identical to a reference amino acid sequence selected from the group consisting of:
(a) amino acids 142 to 211 of SEQ ID NO:2;
(b) amino acids 2 to 335 of SEQ ID NO:2;
(c) amino acids 1 to 335 of SEQ ID NO:2; and
(d) the complete amino acid sequence encoded by the cDNA clone contained in ATCC Deposit No. 203072; wherein said first amino acid sequence regulates Prostate-Specific Antigen (PSA) gene expression.

17. The isolated polynucleotide of claim 16, wherein said first amino acid sequence is at least 99% identical to amino acids 142 to 211 of SEQ ID NO:2.

18. The isolated polynucleotide of claim 17, comprising a nucleic acid encoding amino acids 142 to 211 of SEQ ID NO:2.

19. The isolated polynucleotide of claim 16, wherein said first amino acid sequence is at least 99% identical to amino acids 2 to 335 of SEQ ID NO:2.

20. The isolated polynucleotide of claim 19, comprising a nucleic acid encoding amino acids 2 to 335 of SEQ ID NO:2.

21. The isolated polynucleotide of claim 16, wherein said first amino acid sequence is at least 99% identical to amino acids 1 to 335 of SEQ ID NO:2.

22. The isolated polynucleotide of claim 21, comprising a nucleic acid encoding amino acids 1 to 335 of SEQ ID NO:2.

23. The isolated polynucleotide of claim 16, wherein said first amino acid sequence is at least 99% identical to the complete amino acid sequence encoded by the cDNA clone contained in ATCC Deposit No. 203072.

24. The isolated polynucleotide of claim 23, comprising a nucleic acid encoding the complete amino acid sequence encoded by the cDNA clone contained in ATCC Deposit No. 203072.

25. The isolated polynucleotide of claim 16, further comprising a nucleotide sequence heterologous to said nucleic acid.

26. A method of producing a vector comprising inserting the isolated polynucleotide of claim 16 into a vector.

27. A vector comprising the isolated polynucleotide of claim 16.

28. The vector of claim 27, wherein said polynucleotide comprises a nucleotide sequence heterologous to said nucleic acid.

29. A host cell comprising the isolated polynucleotide of claim 16.

30. The host cell of claim 29, wherein said polynucleotide comprises a nucleotide sequence heterologous to said nucleic acid.

31. A method of producing a polypeptide comprising culturing the host cell of claim 29 under conditions such that a polypeptide is expressed, and recovering said polypeptide.

32. An isolated polynucleotide comprising a nucleic acid encoding a fragment of SEQ ID NO:2 or a fragment of the amino acid sequence encoded by the cDNA clone in ATCC Deposit No. 203072; wherein said fragment regulates Prostate-Specific Antigen (PSA) gene expression; or the complement of said nucleic acid.

33. The isolated polynucleotide of claim 32, further comprising a nucleotide sequence heterologous to said nucleic acid.

34. A method of producing a vector comprising inserting the isolated polynucleotide of claim 32 into a vector.

35. A vector comprising the isolated polynucleotide of claim 32.

36. The vector of claim 35, wherein said polynucleotide comprises a nucleotide sequence heterologous to said nucleic acid.

37. A host cell comprising the isolated polynucleotide of claim 32.

38. The host cell of claim 37, wherein said polynucleotide comprises a nucleotide sequence heterologous to said nucleic acid.

39. A method of producing a polypeptide comprising culturing the host cell of claim 37 under conditions such that a polypeptide is expressed, and recovering said polypeptide.

40. An isolated polynucleotide comprising a nucleic acid selected from the group consisting of:
(a) a nucleic acid encoding amino acids 21 to 29 of SEQ ID NO:2;
(b) a nucleic acid encoding amino acids 38 to 46 of SEQ ID NO:2;
(c) a nucleic acid encoding amino acids 46 to 54 of SEQ ID NO:2;
(d) a nucleic acid encoding amino acids 66 to 74 of SEQ ID NO:2;
(e) a nucleic acid encoding amino acids 75 to 83 of SEQ ID NO:2;
(f) a nucleic acid encoding amino acids 84 to 92 of SEQ ID NO:2;
(g) a nucleic acid encoding amino acids 130 to 138 of SEQ ID NO:2;
(h) a nucleic acid encoding amino acids 146 to 154 of SEQ ID NO:2;
(i) a nucleic acid encoding amino acids 165 to 173 of SEQ ID NO:2;
(j) a nucleic acid encoding amino acids 178 to 186 of SEQ ID NO:2;
(k) a nucleic acid encoding amino acids 192 to 200 of SEQ ID NO:2;
(l) a nucleic acid encoding amino acids 215 to 222 of SEQ ID NO:2;
(m) a nucleic acid encoding amino acids 229 to 237 of SEQ ID NO:2; and
(n) a nucleic acid encoding amino acids 234 to 242 of SEQ ID NO:2.

41. The isolated polynucleotide of claim 40, wherein said nucleic acid is (a).

42. The isolated polynucleotide of claim 40, wherein said nucleic acid is (b).

43. The isolated polynucleotide of claim 40, wherein said nucleic acid is (c).

44. The isolated polynucleotide of claim 40, wherein said nucleic acid is (d).

45. The isolated polynucleotide of claim 40, wherein said nucleic acid is (e).

46. The isolated polynucleotide of claim 40, wherein said nucleic acid is (f).

47. The isolated polynucleotide of claim 40, wherein said nucleic acid is (g).

48. The isolated polynucleotide of claim 40, wherein said nucleic acid is (h).

49. The isolated polynucleotide of claim 40, wherein said nucleic acid is (i).

50. The isolated polynucleotide of claim 40, wherein said nucleic acid is (j).

51. The isolated polynucleotide of claim 40, wherein said nucleic acid is (k).

52. The isolated polynucleotide of claim 40, wherein said nucleic acid is (l).

53. The isolated polynucleotide of claim 40, wherein said nucleic acid is (m).

54. The isolated polynucleotide of claim 40, wherein said nucleic acid is (n).

55. An isolated polynucleotide comprising a nucleic acid encoding at least 100 contiguous amino acids of SEQ ID NO:2.

56. The isolated polynucleotide of claim 55, comprising a nucleic acid encoding at least 150 contiguous amino acids of SEQ ID NO:2.

57. The isolated polynucleotide of claim 55, further comprising a nucleotide sequence heterologous to said nucleic acid.

58. A method of producing a vector comprising inserting the isolated polynucleotide of claim 55 into a vector.

59. A vector comprising the isolated polynucleotide of claim 55.

60. The vector of claim 59, wherein said polynucleotide comprises a nucleotide sequence heterologous to said nucleic acid.

61. A host cell comprising the isolated polynucleotide of claim 55.

62. The host cell of claim 61, wherein said polynucleotide comprises a nucleotide sequence heterologous to said nucleic acid.

63. A method of producing a polypeptide comprising culturing the host cell of claim 61 under conditions such that a polypeptide is expressed, and recovering said polypeptide.

64. A composition comprising the isolated polynucleotide of claim 55.

65. An isolated polynucleotide comprising a first nucleic acid which hybridizes in a wash solution consisting of 0.1×SSC at 65° C. over the entire length of the first nucleic acid to a second nucleic acid selected from the group consisting of:
(a) a nucleic acid consisting of the coding region of the cDNA clone contained in ATCC Deposit No. 203072 or the complement thereof; and
(b) a nucleic acid consisting of the coding region of SEQ ID NO:1 or the complement thereof;
wherein said first nucleic acid is 600 or more nucleotides long.

66. The isolated polynucleotide of claim 65, further comprising a nucleotide sequence heterologous to said first nucleic acid.

67. A method of producing a vector comprising inserting the isolated polynucleotide of claim 65 into a vector.

68. A vector comprising the isolated polynucleotide of claim 65.

69. The vector of claim 68, wherein said polynucleotide comprises a nucleotide sequence heterologous to said first nucleic acid.

70. A host cell comprising the isolated polynucleotide of claim 65.

71. The host cell of claim 70, wherein said polynucleotide comprises a nucleotide sequence heterologous to said first nucleic acid.

72. An isolated polynucleotide comprising a nucleic acid encoding an amino acid sequence from position m to position n of SEQ ID NO:2, wherein m is an integer from 2 to 236, n is an integer from 243 to 335; and m is less than n.

73. The isolated polynucleotide of claim 72, wherein said nucleic acid encodes an amino acid sequence at least 20 amino acids in length.

74. The isolated polynucleotide of claim 72, further comprising a nucleotide sequence heterologous to said nucleic acid.

75. A method of producing a vector comprising inserting the isolated polynucleotide of claim 72 into a vector.

76. A vector comprising the isolated polynucleotide of claim 72.

77. The vector of claim 76, wherein said polynucleotide comprises a nucleotide sequence heterologous to said first nucleic acid.

78. A host cell comprising the isolated polynucleotide of claim 72.

79. The host cell of claim 78, wherein said polynucleotide comprises a nucleotide sequence heterologous to said first nucleic acid.

80. A method of producing a polypeptide comprising culturing the host cell of claim 78 under conditions such that a polypeptide is expressed, and recovering said polypeptide.

81. A polynucleotide comprising a nucleic acid fused in frame to a nucleotide sequence heterologous to SEQ ID NO:1, wherein said heterologous nucleotide sequence encodes a heterologous polypeptide, and wherein said nucleic acid is selected from the group consisting of:
(a) a nucleic acid encoding amino acids 279 to 287 of SEQ ID NO:2;
(b) a nucleic acid encoding amino acids 292 to 300 of SEQ ID NO:2;
(c) a nucleic acid encoding amino acids 317 to 325 of SEQ ID NO:2;
(d) a nucleic acid encoding amino acids 239 to 247 of SEQ ID NO:2;
(e) a nucleic acid encoding amino acids 272 to 280 of SEQ ID NO:2; and
(f) a nucleic acid encoding amino acids 248 to 331 of SEQ ID NO:2.

82. The isolated polynucleotide of claim 81, wherein said nucleic acid encodes amino acids 292 to 300 of SEQ ID NO:2.

83. The isolated polynucleotide of claim 81, wherein said nucleic acid encodes amino acids 317 to 325 of SEQ ID NO:2.

84. The isolated polynucleotide of claim 81, wherein said nucleic acid encodes amino acids 239 to 247 of SEQ ID NO:2

85. The isolated polynucleotide of claim 81, wherein said nucleic acid encodes amino acids 272 to 280 of SEQ ID NO:2.

86. The isolated polynucleotide of claim 81, wherein said nucleic acid encodes amino acids 248 to 331 of SEQ ID NO:2.

87. A method of producing a vector comprising inserting the isolated polynucleotide of claim 81 into a vector.

88. A vector comprising the isolated polynucleotide of claim 81.

89. The vector of claim 88, wherein said polynucleotide comprises a nucleotide sequence heterologous to said nucleic acid.

90. A host cell comprising the isolated polynucleotide of claim 81.

91. The host cell of claim 81, wherein said polynucleotide comprises a nucleotide sequence heterologous to said nucleic acid.

92. A method of producing a polypeptide comprising culturing the host cell of claim 81 under conditions such that a polypeptide is expressed, and recovering said polypeptide.

93. An isolated polynucleotide comprising a nucleic acid encoding at least 60 contiguous amino acids of SEQ ID NO:2;
wherein said nucleic acid is associated with a heterologous sequence selected from the group consisting of a promoter, a site for transcription initiation, a site for transcription termination, an enhancer, a Kozak sequence, an operator and a ribosome binding site.

94. The isolated polynucleotide of claim 93, wherein said nucleic acid encodes at least 70 contiguous amino acids of SEQ ID NO:2.

95. The isolated polynucleotide of claim 94, wherein said nucleic acid encodes at least 80 contiguous amino acids of SEQ ID NO:2.

96. A method of producing a vector comprising inserting the isolated polynucleotide of claim 93 into a vector.

97. A vector comprising the isolated polynucleotide of claim 93.

98. A host cell comprising the isolated polynucleotide of claim 93.

99. A method of producing the polypeptide encoded by the polynucleotide of claim 93, comprising culturing a host cell comprising said polynucleotide under conditions such that said polypeptide is expressed, and recovering said polypeptide.

100. A polynucleotide comprising a nucleic acid fused to a nucleotide sequence heterologous to SEQ ID NO:1, wherein said heterologous nucleotide sequence encodes a heterologous polypeptide, and wherein said nucleic acid is selected from the group consisting of:
(a) a nucleic acid encoding amino acids 279 to 287 of SEQ ID NO:2;
(b) a nucleic acid encoding amino acids 292 to 300 of SEQ ID NO:2;
(c) a nucleic acid encoding amino acids 317 to 325 of SEQ ID NO:2;
(d) a nucleic acid encoding amino acids 239 to 247 of SEQ ID NO:2;
(e) a nucleic acid encoding amino acids 272 to 280 of SEQ ID NO:2; and
(f) a nucleic acid encoding amino acids 248 to 331 of SEQ ID NO:2;
wherein said nucleic acid is operatively associated with a promoter to express said amino acids.

101. The polynucleotide of claim 100, wherein said nucleic acid encodes amino acids 279 to 287 of SEQ ID NO:2.

102. The polynucleotide of claim 100, wherein said nucleic acid encodes amino acids 292 to 300 of SEQ ID NO:2.

103. The polynucleotide of claim 100, wherein said nucleic acid encodes amino acids 317 to 325 of SEQ ID NO:2.

104. The polynucleotide of claim 100, wherein said nucleic acid encodes amino acids 239 to 247 of SEQ ID NO:2.

105. The polynucleotide of claim 100, wherein said nucleic acid encodes amino acids 272 to 280 of SEQ ID NO:2.

106. The polynucleotide of claim 100, wherein said nucleic acid encodes amino acids 248 to 331 of SEQ ID NO:2.


SEQUENCE LISTING (15 sequences)

SEQ ID NO: 1 - DNA, 1894 bp, Homo sapiens
gtctgacttcctcccagcacattcctgcactctgccgtgtccacactgccccacagacccagtcctccaagcctgctgccagctccctgcaagcccctcaggttgggccttgccacggtgccagcaggcagccctgggctgggggtaggggactccctacaggcacgcagccctgagacctcagagggccaccccttgagggtggccaggcccccagtggccaacctgagtgctgcctctgccaccagccctgctggcccctggttccgctggccccccagatgcctggctgagacacgccagtggcctcagctgcccacacctcttcccggcccctgaagttggcactgcagcagacagctccctgggcaccaggcagctaacagacacagccgccagcccaaacagcagcggcatgggcagcgccagcccgggtctgagcagcgtatcccccagccacctcctgctgccccccgacacggtgtcgcggacaggcttggagaaggcggcagcgggggcagtgggtctcgagagacgggactggagtcccagtccacccgccacgcccgagcagggcctgtccgccttctacctctcctactttgacatgctgtaccctgaggacagcagctgggcagccaaggcccctggggccagcagtcgggaggagccacctgaggagcctgagcagtgcccggtcattgacagccaagccccagcgggcagcctggacttggtgcccggcgggctgaccttggaggagcactcgctggagcaggtgcagtccatggtggtgggcgaagtgctcaaggacatcgagacggcctgcaagctgctcaacatcaccgcagatcccatggactggagccccagcaatgtgcagaagtggctcctgtggacagagcaccaataccggctgccccccatgggcaaggccttccaggagctggcgggcaaggagctgtgcgccatgtcggaggagcagttccgccagcgctcgcccctgggtggggatgtgctgcacgcccacctggacatctggaagtcagcggcctggatgaaagagcggacttcacctggggcgattcactactgtgcctcgaccagtgaggagagctggaccgacagcgaggtggactcatcatgctccgggcagcccatccacctgtggcagttcctcaaggagttgctactcaagccccacagctatggccgcttcattaggtggctcaacaaggagaagggcatcttcaaaattgaggactcagcccaggtggcccggctgtggggcatccgcaagaaccgtcccgccatgaactacgacaagctgagccgctccatccgccagtattacaagaagggcatcatccggaagccagacatctcccagcgcctcgtctaccagttcgtgcaccccatctgagtgcctggcccagggcctgaaacccgccctcaggggcctctctcctgcctgccctgcctcagccaggccctgagatgggggaaaacgggcagtctgctctgctgctctgaccttccagagcccaaggtcagggaggggcaaccaactgccccagggggatatgggtcctctggggccttcgggaccatggggcaggggtgcttcctcctcaggcccagctgctcccctggaggacagagggagacagggctgctccccaacacctgcctctgaccccagcatttccagagcagagcctacagaagggcagtgactcgacaaaggccacaggcagtccaggcctctctctgctccatccccctgcctcccattctgcaccacacctggcatggtgcagggagacatctgcacccctgagttgggcagccaggagtgcccccgggaatggataataaagatactagagaactg

SEQ ID NO: 2 - protein, 335 aa, Homo sapiens
MGSASPGLSSVSPSHLLLPPDTVSRTGLEKAAAGAVGLERRDWSPSPPATPEQGLSAFYLSYFDMLYPEDSSWAAKAPGASSREEPPEEPEQCPVIDSQAPAGSLDLVPGGLTLEEHSLEQVQSMVVGEVLKDIETACKLLNITADPMDWSPSNVQKWLLWTEHQYRLPPMGKAFQELAGKELCAMSEEQFRQRSPLGGDVLHAHLDIWKSAAWMKERTSPGAIHYCASTSEESWTDSEVDSSCSGQPIHLWQFLKELLLKPHSYGRFIRWLNKEKGIFKIEDSAQVARLWGIRKNRPAMNYDKLSRSIRQYYKKGIIRKPDISQRLVYQFVHPI

SEQ ID NO: 3 - protein, 114 aa, Drosophila melanogaster
TNASNGGTATVKRPNGGRTGGGGSHIHLWQFLKELLASPQVNGTAIRWIDRSKGIFKIEDSVRVAKLWGRRKNRPAMNYDKLSRSIRQYYKKGIMKKTERSQRLVYQFCHPYSQ

SEQ ID NO: 4 - DNA, 733 bp, Homo sapiens
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 5 - protein, 5 aa, Homo sapiens
  UNSURE (3): May be any amino acid
WSXWS

SEQ ID NO: 6 - DNA, 86 bp, Artificial Sequence
  Description of Artificial Sequence DNA primer
gcgcctcgagatttccccgaaatctagatttccccgaaatgatttccccgaaatgatttccccgaaatatctgccatctcaattag

SEQ ID NO: 7 - DNA, 27 bp, Artificial Sequence
  Description of Artificial Sequence DNA primer
gcggcaagctttttgcaaagcctaggc

SEQ ID NO: 8 - DNA, 271 bp, Artificial Sequence
  Description of Artificial Sequence PCR fragment
ctcgagatttccccgaaatctagatttccccgaaatgatttccccgaaatgatttccccgaaatatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 9 - DNA, 32 bp, Homo sapiens
gcgctcgagggatgacagcgatagaaccccgg

SEQ ID NO: 10 - DNA, 31 bp, Homo sapiens
gcgaagcttcgcgactccccggatccgcctc

SEQ ID NO: 11 - DNA, 12 bp, Artificial Sequence
  Description of Artificial Sequence DNA primer
ggggactttccc

SEQ ID NO: 12 - DNA, 73 bp, Artificial Sequence
  Description of Artificial Sequence DNA Primer
gcggcctcgaggggactttcccggggactttccggggactttccgggactttccatcctgccatctcaattag

SEQ ID NO: 13 - DNA, 256 bp, Artificial Sequence
  Description of Artificial Sequence PCR fragment
ctcgaggggactttcccggggactttccggggactttccgggactttccatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 14 - DNA, 20 bp, Artificial Sequence
  Description of Artificial Sequence DNA Primer
caaagttgtcatggatgacc

SEQ ID NO: 15 - DNA, 18 bp, Artificial Sequence
  Description of Artificial Sequence DNA Primer
ccatggagaaggctgggg